(19) United States
(12) Patent Application Publication
(10) Pub. No.: US 2010/0233678 A1
(43) Pub. Date: Sep. 16, 2010

(54) TUNABLE AFFINITY LIGANDS FOR THE SEPARATION AND DETECTION OF TARGET SUBSTANCES

(76) Inventor: Leslie C. Beadling, South Plainfield, NJ (US)

Correspondence Address: David M McConoughey, P O Box 8477, New York, NY, 10116-8477, US

(21) Appl. No.: 12/734,639

(22) PCT Filed: Nov. 13, 2008
(86) PCT No.: PCT/US2008/012724

§ 371 Date: May 13, 2010

(30) Foreign Application Priority Data
Nov. 13, 2007 (US) 61002827
Nov. 13, 2007 (US) 61002828

(51) Int. Cl. G01N 33/53; C12N 7/02; C07K 14/00; C07K 2/00; C07H 1/06; C07K 14/575; C12M 1/00; C07H 19/00; C12M 1/34; C12Q 1/70; G01N 30/00

(52) U.S. Cl. 435/5; 435/239; 530/350; 530/300; 536/1.11; 530/399; 435/308.1; 536/22.1; 435/287.1; 435/7.8; 436/501; 422/69

(57) ABSTRACT

Conformationally tunable affinity ligands are rationally designed and selected for the ability to switch under operator-defined environmental conditions between or among structurally distinct states that have different affinities for a given target substance. Tunable affinity ligands are incorporated into reagents, separation media, assays, sensors, devices, kits and systems for sorting, separating, detecting, sensing, quantifying, identifying and monitoring target substances. Applications include biomedical research, diagnostics, drug discovery, bioproduction and processing and environmental, industrial, chemical, agricultural and military use.

### TECHNICAL FIELD

This invention relates to conformationally tunable ligands that are rationally designed and selected for the ability to switch under defined environmental conditions between or among structurally distinct states that have different affinities for a given target substance. These tunable ligands can be used for the separation, detection and monitoring of target substances, e.g., molecules, multimolecular and supramolecular complexes, microorganisms, viruses and cells, for applications including, e.g., 1) sorting and purification of substances from complex mixtures, 2) detection and quantification of diagnostic analytes in biological, environmental, industrial, chemical and agricultural samples and systems, 3) resolving molecular signatures of biological differentiation, development and disease, 4) characterization, standardization and validation of specialty chemicals, diagnostic reagents, biologicals and drugs and 5) drug discovery.

### SUMMARY OF THE INVENTION

In an embodiment of the present invention, a medium for separating a target substance from a mixture of substances comprises a nucleotide-containing tunable affinity ligand (TAL) within a reaction mixture, said tunable affinity ligand existing in a first conformational state having a quantifiable first affinity for the target substance under a first set of reaction conditions and a second conformational state having a quantifiable second affinity for the target substance under a second set of reaction conditions wherein the first affinity is measurably different from the second affinity.

In another embodiment of the present invention, a device for isolating target substances from a sample comprises:
a) a nucleotide-containing tunable affinity ligand capable of existing in a target-binding state and a target-nonbinding state;
b) means for delivering the sample to the tunable affinity ligand to form a reaction mixture in which the tunable affinity ligand exists in the target-binding state;
c) means for partitioning ligand-target complexes from other substances in the reaction mixture;
d) means for converting the tunable affinity ligand from the target-binding state to the target-nonbinding state; and
e) means for partitioning unbound target molecules from ligand-bound target molecules.

In another embodiment of the present invention, a kit for separating a target substance from a sample comprises a buffer-responsive nucleotide-containing tunable affinity ligand, a binding buffer and a releasing buffer wherein the tunable affinity ligand switches between a target-binding state in the presence of the binding buffer and a target-nonbinding state in the presence of the releasing buffer.

In another embodiment of the present invention, a system for separating a target substance from a sample comprises:
a) a processing reservoir containing a separation reagent;
b) input means for delivering the sample to the processing reservoir;
c) output means for removing the target substance from the processing reservoir;
d) a first buffer solution; and
e) a second buffer solution;
wherein the separation reagent is a nucleotide-containing tunable affinity ligand that exists in a first conformational state having a quantifiable first affinity for the target substance under a first set of reaction conditions and a second conformational state having a quantifiable second affinity for the target substance under a second set of reaction conditions wherein the first affinity is measurably different from the second affinity.

In another embodiment of the present invention, a method of purifying a target substance from a sample comprises:
a) contacting the sample with an environmentally-sensitive nucleotide containing tunable affinity ligand under a first environmental condition under which the tunable affinity ligand binds to the target substance to form a ligand-target complex;
b) partitioning the ligand-target complex from nontarget substances in the sample; and
c) releasing the target substance from the ligand-target complex by exposing the ligand-target complex to a second environmental condition;
   wherein
i) the tunable affinity ligand reversibly partitions between a first conformational state having a first affinity for the target substance under the first environmental condition and a second conformational state having a second affinity for the target substance under the second environmental condition; and
ii) the first affinity is measurably different from the second affinity.

In another embodiment of the present invention, a method of separating a first substance in a sample from a second substance in the sample comprises:
a) contacting the sample with a nucleotide-containing tunable affinity ligand immobilized on a support immersed in a binding buffer;
b) incubating the sample with the immobilized tunable affinity ligand for a sufficient contact time to allow the immobilized tunable affinity ligand to bind the first substance to form an immobilized ligand-substance complex;
c) performing a rinsing step to remove the second substance;
d) performing at least one elution step to dissociate the first substance from the ligand of the immobilized ligand-substance complex; and
e) collecting at least one product of the at least one elution step;
   wherein
i) said at least one product comprises the first substance; and
ii) said at least one elution step causes the tunable affinity ligand to shift from a first conformational state that favors association of immobilized ligand-substance complexes to a second (or third or fourth, etc.) conformational state that favors dissociation of immobilized ligand-substance complex.

In another embodiment of the present invention, a separation medium comprises a support-bound plurality of ligands including at least a first ligand and a second ligand, said first ligand being a nucleotide-containing tunable affinity ligand existing in a first state having a quantifiable first affinity for a target substance under a first set of conditions and a second state having a quantifiable second affinity for the target substance under a second set of conditions wherein the first ligand is structurally different from the second ligand.

In another embodiment of the present invention, a reagent for detecting a target substance comprises a nucleotide-containing tunable affinity ligand capable of existing in a first conformational state having a quantifiable first affinity for the target substance under a first set of reaction conditions and a second conformational state having a quantifiable second affinity for the target substance under a first set of reaction conditions wherein the first affinity is measurably different from the second affinity.

In another embodiment of the present invention, a sensor for detecting a target substance comprises a ligand functionally connected to a transducer, said ligand being a nucleotide-containing tunable affinity ligand capable of existing in a first conformational state having a quantifiable first affinity for the target substance under a first set of reaction conditions and a second conformational state having a quantifiable second affinity for the target substance, under a second set of reaction conditions wherein the first affinity is measurably different from the second affinity.

In another embodiment of the present invention, a method for detecting the presence of a target substance comprises:
a) contacting the target substance with target-unbound nucleotide-containing tunable affinity ligands in a first reaction mixture that favors binding of the tunable affinity ligands to the target to form target-bound tunable affinity ligand-receptor complexes;
b) exposing the tunable affinity ligand-receptor complexes to a second reaction mixture that favors dissociation of the tunable affinity ligand-receptor complexes; and
c) detecting a difference in the conformation, properties or affinity state of at least one of the tunable affinity ligands or the tunable affinity ligand-receptor complexes in the target-bound state compared with the target-unbound state.

### DESCRIPTION OF THE DRAWINGS

FIG. 1 presents a comparison of triplex TALs with TTTT loops (solid curve), with hexane loops (dotted curve), and with hexaethylene glycol loops (dashed curve). The binding buffer was 20 mM sodium acetate, pH 5.8, plus 1 mM MgCl₂. The elution buffer was 50 mM Tris, pH 8.3 plus 100 mM KCl. At time 0, a sample containing IgG was injected onto the column.

FIG. 2 presents a comparison of a serum sample run on a Protein A-Sepharose column (a) and on a TAL Sepharose column (b). For (a) the binding buffer was 20 mM sodium phosphate buffer, pH 7.0, and elution was with a step gradient of 0.1 M citric acid, pH 3.0. For (b) the binding buffer was 20 mM sodium acetate, pH 5.8, plus 1 mM MgCl₂. The elution buffer was 50 mM Tris, pH 8.3 plus 100 mM KCl.

FIG. 3 shows the result of collecting the peak at 10.41 minutes from the TAL column and re-injecting onto a Protein A column (dashed curve). For comparison, the black curve shows the result of injecting serum directly onto the Protein A column. The binding buffer was 20 mM sodium phosphate buffer, pH 7.0, and elution was with a step gradient of 0.1 M citric acid, pH 3.0.

FIG. 4 illustrates IgG subtype separations on a Protein A-Sepharose column (a) and on a TAL Sepharose column (b). For (a) the binding buffer was 20 mM sodium phosphate buffer, pH 7.0, and elution was with a step gradient of 0.1 M citric acid, pH 3.0. For (b) the binding buffer was 20 mM sodium acetate, pH 5.8, plus 1 mM MgCl₂. The elution buffer was 50 mM Tris, pH 8.3 plus 100 mM KCl.

FIG. 5 shows chromatograms from the TAL column of fluorescein-labeled IgG mixed with BSA (solid curves) and with serum (dashed curves). For plot (a) the UV absorbance is monitored at 280 nm. For plot (b) the fluorescence emission is monitored at 528 nm for excitation at 490 nm. The binding buffer was 20 mM sodium acetate, pH 5.8, plus 1 mM MgCl₂. The elution buffer was 50 mM Tris, pH 8.3 plus 100 mM KCl.

FIG. 6 shows the retardation of mouse IgG on the TAL column. The binding buffer was 20 mM sodium acetate, pH 5.8, plus 1 mM MgCl₂. The elution buffer was 50 mM Tris, pH 8.3 plus 100 mM KCl.

FIG. 7 shows the chromatographic separation of thrombin and derivatives using the TTT-aptamer, d(GGTTGGTTTGGTTGG). Buffer A consisted of 125 mM TEAA, 10 mM KCl, pH 6.5. Buffer B consisted of 500 mM LiCl, 10 mM TEAA. The protein was added in buffer A, followed by 4.5 min elution (flow rate 0.9 ml/min) with buffer A. The column was then eluted with a gradient of 0-100% buffer B over 4.5 min. Finally, the column was eluted with buffer B for an additional 9.5 min.

FIG. 8 shows the chromatographic separation of thrombin and derivatives using a nondenaturing anti-thrombin TAL with a TTT loop, and inosine bases substituted for guanines. The TAL sequence is d(IGTTGGTTTIGTTGG). Note the improved resolution of the alpha-thrombin from the other proteins. Conditions are as in FIG. 7.

FIG. 9 features theoretical results for a model where the buffer flows into a stirred 1 ml vessel at 0.5 ml/min. From 0-10 minutes, the buffer is 50 mM KCl. From 10-20 minutes a linear gradient of buffer B (0.5 M LiCl) is applied. From 20 minutes to the end of the run, the buffer flowing into the column is buffer B. (a) K₂^{obs}=0.0001 in pure buffer A (50 mM KCl) (b) K₂^{obs}=1.0 in pure buffer A.

FIG. 10 shows a contour plot of intensities (red highest, blue lowest) for a model 4×4 array of labeled hairpin-quadruplex TALs, with K₂^{T} values that are arrayed according to:

$$[ \begin{matrix}0.075 & 0.75 & 7.5 & 75 \\ 0.05 & 0.5 & 5 & 50 \\ 0.025 & 0.25 & 2.5 & 25 \\ 0.01 & 0.1 & 1 & 10\end{matrix} ]$$

where K₂^{T} is the thermodynamic equilibrium constant for the quadruplex-hairpin transition, defined as described in Example 6 below, for standard salt conditions. In this plot, the x-axis is arranged according to K₃^{T} values (intrinsic protein binding affinity), whereas the y-axis shows a different fraction of K⁺ containing buffer (here, buffer A=10 mM KCl, buffer B=100 mM LiCl).

FIG. 11 provides examples of TALs that partition between structured conformations. (A) Triplex-three-way junction, (B) Quadruplex-triplex, and (C) Quadruplex-three-way Junction.

FIG. 12 provides an example of a TAL that partitions among three structured conformations: triplex, three-way junction, and quadruplex.

FIG. 13 illustrates the circular dichroism (CD) versus temperature plot for HPL DNA with 100 mM sodium phosphate buffer and 100 mM KCl. As shown, HPL DNA was 100% stabilized at 20° C. (diamond) and completely destabilized at 80-90° C. (pluses). At approximately 50° C. (X), the HPL DNA was 50% dissociated by the increased temperature.

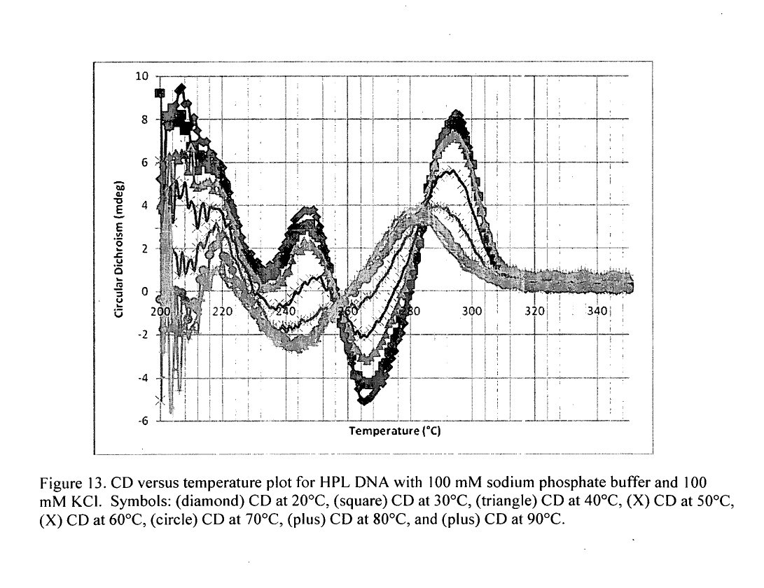

### DESCRIPTION OF EMBODIMENTS OF THE INVENTION

In the present invention, TALs capable of existing in a plurality of states are used for purposes of detecting, separating, profiling and purifying target substances, including, e.g., molecules, macromolecular complexes, organelles, prokaryotic and eukaryotic cells and viruses. TALs disclosed herein may be designed, formatted and used in methods, compositions and articles of manufacture, including kits, devices, and systems.

In an embodiment of the present invention, a medium for separating a target substance from a mixture of substances, said medium comprises a tunable affinity ligand within a reaction mixture, said tunable affinity ligand existing in a first conformational state having a quantifiable first affinity for the target substance under a first set of reaction conditions and a second conformational state having a quantifiable second affinity for the target substance under a second set of reaction conditions wherein the first affinity is measurably different from the second affinity.

In another embodiment of the present invention, a device for isolating target substances from a sample, said device comprises:
a) tunable affinity ligand capable of existing in a target-binding state and a target-nonbinding state;
b) means for delivering the sample to the tunable affinity ligand to form a reaction mixture in which the tunable affinity ligand exists in the target-binding state;
c) means for partitioning ligand-target complexes from other substances in the reaction mixture;
d) means for converting the tunable affinity ligand from the target-binding state to the target-nonbinding state; and
e) means for partitioning unbound target molecules from ligand-bound target molecules.

In another embodiment of the present invention, a kit for separating a target substance from a sample comprises a buffer-responsive tunable affinity ligand, a binding buffer and a releasing buffer wherein the tunable affinity ligand switches between a target-binding state in the presence of the binding buffer and a target-nonbinding state in the presence of the releasing buffer.

In another embodiment of the present invention, a system for separating a target substance from a sample comprises:
a) a processing reservoir containing a separation reagent;
b) input means for delivering the sample to the processing reservoir;
c) output means for removing the target substance from the processing reservoir;
d) a first buffer solution; and
e) a second buffer solution;
wherein the separation reagent is a tunable affinity ligand that exists in a first conformational state having a quantifiable first affinity for the target substance under a first set of reaction conditions and a second conformational state having a quantifiable second affinity for the target substance under a second set of reaction conditions wherein the first affinity is measurably different from the second affinity.

In another embodiment of the present invention, a method of purifying a target substance from a sample comprises:
a) contacting the sample with an environmentally-sensitive tunable affinity ligand under a first environmental condition under which the tunable affinity ligand binds to the target substance to form a ligand-target complex;
b) partitioning the ligand-target complex from nontarget substances in the sample; and
c) releasing the target substance from the ligand-target complex by exposing the ligand-target complex to a second environmental condition;
   wherein
i) the tunable affinity ligand reversibly partitions between a first conformational state having a first affinity for the target substance under the first environmental condition and a second conformational state having a second affinity for the target substance under the second environmental condition; and
ii) the first affinity is measurably different from the second affinity.

In another embodiment of the present invention, a method of separating a first substance in a sample from a second substance in the sample comprises:
a) contacting the sample with a tunable affinity ligand immobilized on a support immersed in a binding buffer;
b) incubating the sample with the immobilized tunable affinity ligand for a sufficient contact time to allow the immobilized tunable affinity ligand to bind the first substance to form an immobilized ligand-substance complex;
c) performing a rinsing step to remove the second substance;
d) performing at least one elution step to dissociate the first substance from the ligand of the immobilized ligand-substance complex; and
e) collecting at least one product of the at least one elution step;
   wherein
i) said at least one product comprises the first substance; and
ii) said at least one elution step causes the tunable affinity ligand to shift from a first conformational state that favors association of immobilized ligand-substance complexes to a second (or third or fourth, etc.) conformational state that favors dissociation of immobilized ligand-substance complexes.

In another embodiment of the present invention, a separation medium comprises a support-bound plurality of ligands including at least a first ligand and a second ligand, said first ligand being a tunable affinity ligand existing in a first state having a quantifiable first affinity for a target substance under a first set of conditions and a second state having a quantifiable second affinity for the target substance under a second set of conditions wherein the first ligand is structurally different from the second ligand.

In another embodiment of the present invention, a reagent for detecting a target substance comprises a tunable affinity ligand capable of existing in a first conformational state having a quantifiable first affinity for the target substance under a first set of reaction conditions and a second conformational state having a quantifiable second affinity for the target substance under a second set of reaction conditions wherein the first affinity is measurably different from the second affinity.

In another embodiment of the present invention, a sensor for detecting a target substance comprises a ligand functionally connected to a transducer, said ligand being a tunable affinity ligand capable of existing in a first conformational state having a quantifiable first affinity for the target substance under a first set of reaction conditions and a second conformational state having a quantifiable second affinity for the target substance under a first set of reaction conditions wherein the first affinity is measurably different from the second affinity.

In another embodiment of the present invention, a method for detecting the presence of a target substance comprises:
a) contacting the target substance with target-unbound nucleotide-containing tunable affinity ligands in a first reaction mixture that favors binding of the tunable affinity ligands to form target-bound tunable affinity ligand-receptor complexes;
b) exposing the tunable affinity ligand-receptor complexes to a second reaction mixture that favors dissociation of the tunable affinity ligand-receptor complexes; and
c) detecting a difference in the conformation, properties or affinity state of at least one of the tunable affinity ligands or the tunable affinity ligand-receptor complexes in the target-bound state compared with the target-unbound state. As context, affinity-based ligands for molecular and cellular separations and detection include antibodies, peptides, proteins, lectins, nucleic acid aptamers and low molecular weight organic and inorganic molecules such as intercalating agents and dyes. Unlike the conformationally tunable ligands disclosed herein, environmentally induced changes in the affinity of prior art affinity ligands are accompanied by nonspecific and/or undefined changes in the cognate target substance. The affinity of an antibody for its target antigen, for example, is pH-sensitive. However, under pH-dependent affinity-altering conditions, both the antibody and antigen are subject to perturbations in structure and stability Such perturbations are disadvantageous in affinity separations and specific binding assays, as they may damage target molecules and cells, cause artifacts in experimental results and call into question the reliability of associated preparative and analytical procedures.

### GLOSSARY

The term “affine conformation” means a multiparameter distribution of the atoms conferring affinity on an affinity state, where parameters include, e.g., the spatial positioning of the atoms between and among one another within the conformation. Conformation is determined by structural and/or functional analytical techniques, e.g., by chemical, physical, and/or biological analytical methodologies that identify a particular multiparameter distribution of the atoms. Structural information can be obtained, e.g., by NMR spectroscopy, UV spectroscopy, CD spectroscopy, calorimetry, hydrodynamic, chromatography and electrophoresis. The affinity of a particular conformation can be measured by a variety of techniques for detecting and quantifying molecular interactions, including ligand-receptor binding assays such as filtration assays, immunoassays, polarization assays and the like. Illustrative examples of such chemical methodologies, physical methodologies, and chemical and physical methodologies are described.

The term “affine” means having the property of affinity.

The term “affinity” means tendency to associate (“bind”) noncovalently. Noncovalent refers to interactions that do not involve the formation of covalent chemical bonds. Covalent chemical bonds are bonds between atoms that involve the sharing of electron pairs. Covalent bonds are the bonds that hold atoms together as distinct molecules. For example, the hexane molecule comprises 6 carbon atoms and 14 hydrogen atoms that are held together by 5 carbon-carbon covalent bonds and 14 carbon-hydrogen covalent bonds. Noncovalent associations involve associations between or among molecules, and may involve a variety of noncovalent forces including hydrogen bonds, Van der Waals forces, or electrostatic forces. If a ligand has an affinity for a particular target, that means there is a favorable tendency for the ligand to associate specifically and noncovalently with the target to form a complex or complexes. The magnitude of the affinity may be defined by an equilibrium constant for complex formation or equilibrium constants for complex formation or by the corresponding free energy of complex formation or the free energies of complex formation. By rigorous thermodynamic convention, affinity is expressed in energy units per mole (e.g. kilojoules/mole or kilocalories/mole) for free energies or in dimensionless units for equilibrium constants. According to this convention, the free energy of a binding event describes the heat given off or taken up during the association of defined molar amounts of ligand and target. The equilibrium constant for a binding event is given in terms of ratios of the relative activities of unbound and bound forms compared to standard state binding conditions and has dimensionless units. In the limit of an infinitely dilute solution, activities are identical to concentration, and measured equilibrium constants are often expressed in terms of concentration ratios (reference: Kenneth Denbigh, The Principles of Chemical Equilibrium, Cambridge University Press, 1973, London, Chapter 10, pp 292-327.) For practical applications in biochemistry and for the purposes of this application, equilibrium constants are defined in terms of ratios of concentrations of ligands, targets and complexes, and activity coefficient corrections are ignored (see reference: Donald J. Winzor and William H. Sawyer, Quantitative Characterization of Ligand Binding, Wiley-Liss, 1995, New York, N.Y., Chapter 1, pp. 1-11). The affinity of a ligand for its target depends on a number of factors, including, e.g., the conformation of the ligand, the conformation of the target and local environmental parameters such as temperature and ionic conditions, which can strongly influence binding without significantly altering conformation.

The term “affinity ligand” means a ligand having at least a first affinity state characterized by a first measurable affinity for a given target molecule (e.g., a cognate drug, pharmacophore, analyte, peptide, lipid, carbohydrate glycoprotein or viral coat protein) under a first set of conditions and, in the case of a tunable affinity ligand, a second affinity state characterized by a second measurable affinity for the target molecule under a second set of conditions, said first affinity state being capable of changing affinity in response to a defined change in environment or assay conditions.

The term “antibody” means an antigen- or hapten-binding molecule classified as an immunoglobulin, i.e., an antigen- or hapten-binding immunoglobulin. Immunoglobulins may be derived from any one or more of a variety of species, isotypes and subtypes or any combination thereof. They may also be modified through antibody engineering methods known in the art, including conjugation, humanization, chimerization and the like. Species commonly used in biomedical research include but are not limited to mouse, human, rabbit, goat, rat, cow, cat, chicken, dog, donkey, guinea pig, hamster, horse, sheep and swine. For a given species, there is also a variety of immunoglobulin isotypes, and for each isotype there may be more than one subtype. For humans, the dominant isotypes are IgA, IgD, IgE, IgG, and IgM. Subtypes of IgA include IgA1 and IgA2. Subtypes of IgG include IgG1, IgG2, IgG3 and IgG4.

The term “antibody fragment” means a portion of an antibody obtained, e.g., by reduction, enzyme digestion or translation of an antibody-encoding mRNA sequence. Antibody fragments include, for example, isolated Fab, F(ab′), F(ab′)₂ and Fc regions of immunoglobulin molecules.

The term “cognate,” when used in reference to a ligand or target, means the target is specifically recognizable by the ligand or vice versa. A hormone, drug or transmitter that specifically binds to a particular receptor, for example, is referred to as a cognate ligand for that receptor. Conversely, the receptor may be referred to as a cognate receptor for the ligand.

The terms “conformationally tunable multistate affinity ligand” and “multistate affinity ligand” and “tunable affinity ligand” and “TAL” as used herein are synonymous.

The term “conjugate,” when used as a noun, means a covalent complex between at least a first molecule and a second molecule and, when used as a verb, means the act of attaching at least a first molecule to at least a second molecule.

The term “ligand” means a molecule, a molecular complex or a chemically defined part of a molecule or molecular complex that associates specifically and noncovalently with (or “binds to”) a target substance to form a complex involving one or more ligands and one or more target entities. Tunable affinity ligands of the instant invention contain at least one sequence of nucleotides capable of undergoing intramolecular base pairing. The target entity may be a molecule, a portion of a molecule, a macromolecular complex, a biological structure or living organism or a conjugate or complex containing any of these entities and a second molecule, portion of a molecule, complex structure or organism. Target examples include proteins, protein subunits, peptides, nucleic acids, polynucleotides, drugs, hormones, neurotransmitters, carbohydrates, lipids, glycoproteins, lipoproteins, organelles, cell components, cell surfaces, cells, microbes and viruses. Normucleic acid targets include targets that do not contain a sequence of three or more nucleotides and explicitly include individual nucleotides and dinucleotides such as adenosine, flavin adenine dinucleotide, nicotinamide adenine dinucleotide, adenosine diphosphate, adenosine triphosphate and cyclic adenosine monophosphate. In other words, nonnucleic acid targets are neither nucleic acids nor oligonucleotides.

The term “matrix” is another word for “support.”

The term “multistate affinity ligand” as used herein is synonymous with the terms “conformationally tunable multistate affinity ligand” and “tunable affinity ligand” and “TAL.”

The term “nondenaturing,” when used in reference to a tunable affinity ligand means that the cognate target remains essentially unperturbed by interaction with the TAL both structurally and functionally as determined by physical, chemical and biological assays. Not only does the target substance remain intact immediately following interaction with its cognate TAL, it also advantageously retains its structural and functional integrity through repeated cycles of binding and release by the TAL when such repeated cycles are required for preparative or analytical purposes. Further, stability studies of the target substance following interaction with the cognate TAL can be used to show that TAL interaction does not increase the degradation rate of the target substance. This feature is particularly important for biological targets such as proteins, immunoglobulins, glycoproteins, lipoproteins and cells, which have been shown to undergo accelerated degradation following conventional affinity-based purification and analysis procedures, even when the target substance appears to have been intact immediately following ligand interaction.

The terms “nondenaturing tunable affinity ligand” and “nondenaturing TAL” refer to TALs that can be shown to bind and release target substances without perturbing the structure, function and/or stability of the target substances, including fragile biological targets such as proteins, immunoglobulins, glycoproteins, lipids, lipoproteins molecules, cells and the like.

The term “nucleotide” refers to monomers and sequences comprising natural, synthetic and nonnatural nucleic acid molecules and includes nucleotide bases, analogs, modified bases and other monomers that can be substituted for nucleotide bases during the synthesis of oligonucleotides. Nucleotides include groups of nucleotide monomers comprising oligonucleotides. Any compound containing a heterocyclic compound bound to a phosphorylated sugar by an N-glycosyl link or any monomer capable of complementary base pairing or any polymer capable of hybridizing to nucleic acid molecule is considered a nucleotide as the term is used herein, including nucleotides comprising backbone modifications, abasic regions, spacers, linkers, hinge regions, bridges, space-/charge-modifiers and the like.

The term “nucleotide-containing,” when used in reference to a tunable affinity ligand, means that the tunable affinity ligand contains a sequence of at least three nucleotides, advantageously a sequence capable of intramolecular base pairing.

The term “oligonucleotide” means a naturally occurring, synthetic or nonnaturally occurring polymer of nucleotides, preferably a polymer comprising at least three nucleotides that is capable of intramolecular or intermolecular base pairing and/or participation in formation of duplex, triplex, tetraplex, quadruplex, junction and/or higher order nucleotide structures. Oligonucleotides may be, for example and without limitation, single-stranded, double-stranded, partially single-stranded, partially double-stranded, multi-stranded or partially multi-stranded ribonucleic, deoxyribonucleic, peptide or mixed nucleic acids that may include backbone modifications, heteroduplexes, chimeric structures and the like as well as nucleotides conjugated to one or more normucleotide molecules. Although oligonucleotides of the instant invention typically range in length from about five nucleotides to about 100 nucleotides, they may contain hundreds or even thousands of nucleotides. There is no intrinsic upper limit. Monomeric and dimeric nucleotides such as biological cofactors, messengers and metabolites, e.g., adenosine, AMP, ADP, ATP, cAMP, NAD, NADH, NADH2, FAD, FADH and FADH2, are not considered oligonucleotides as the term is used herein.

The term “polynucleotide” refers to a sequence of nucleotides.

The term “reaction mixture,” when used in reference to a tunable affinity ligand means a solution containing or contacting a tunable affinity ligand wherein the composition of the solution can be varied under operator-, instrument- or device-dependent control.

The term “reagent,” when used in reference to molecular constructs of the instant invention, means a synthetic preparation comprising a tunable affinity ligand.

The term “receptor” means a cognate binding partner of a ligand and is used as an alternative to the term “target” in some contexts, e.g., reference to ligand-receptor interactions.

The term “self-reporting,” when used in reference to a tunable affinity ligand, means that the state of the ligand can be determined without separation or washing steps and is typically used in the context of discriminating target-bound from target-unbound states of the ligand as is particularly useful in analytical procedures, e.g., specific binding assays.

The term “specific binding” refers to noncovalent interaction between a ligand and a target substance that can be inhibited by structural analogs of the ligand or target substance.

The term “specific binding assay” refers to analytical procedures for the detection, monitoring and/or quantification of a target substance in a reaction mixture.

The term “sensor” means a device capable of sensing, detecting, measuring, monitoring, determining or quantifying the presence or amount of one or more substances or events and includes, without limitation, mechanical sensors, force and mass sensors, acoustic sensors, chemical sensors, biosensors, electrochemical sensors, optical sensors, electromagnetic sensors, electrical sensors, electronic sensors, optoelectronic sensors and, photodetectors. Advantageously, sensors have the useful property, given suitable recognition and transduction components, to reversibly and sequentially detect both increases and/or decreases in the amount of target substance in a subject, specimen or sample, e.g., by monitoring the binding and release of a target to its cognate ligand.

The term “support” means a three-dimensional material, the surface of which may be modified, e.g., by one or more covalent or high-affinity noncovalent chemistries or physical or chemical deposition methods designed to attach, immobilize or localize ligands or targets for separation, detection, sensing or other applications.

The term “TAL” means “tunable affinity ligand” and is synonymous with the terms “multistate affinity ligand” and “conformationally tunable multistate affinity ligand” as used herein,

The term “target” means a natural, synthetic, biological or nonbiological substance, material, molecule, complex, particle or structure and may be referred to as a “receptor” in the context of ligand-receptor interactions. Biological targets include, for example and without limitation, amino acids, proteins, peptides, hormones, transmitters, pharmacophores, drugs, hormones, metabolites, carbohydrates, glycoproteins, viruses, microbes, pathogens, organelles, cells, tissues, organs and organisms. Protein- and peptide-based targets include post-translationally modified species resulting from, e.g., cleavage or degradation to short peptides or amino acids, phosphorylation, alkylation, deamidation, glycosylation, polyglutamylation, acetylation, serinization, tyrosination, excision of amino acids and modifications resulting from treatment of synthesized peptides or proteins. Nonbiological targets include, for example and without limitation, industrial polymers, dyes, petrochemicals, specialty chemicals, hazardous waste materials, pesticides, herbicides, synthetic toxins and synthetic nanomaterials.

The term “target-binding,” when used in reference to the state of a tunable affinity ligand, means a conformational state of the ligand that favors ligand-target complex formation in the presence of a target substance.

The term “target-nonbinding,” when used in reference to the state of a tunable affinity ligand, means a conformational state of the ligand that favors the unbound form of the ligand in the presence of a target substance.

The term “transducer” means a device, surface or system capable of converting the mass or energy of ligand-target binding or a change in ligand conformational or a change in the activity of the ligand, target or ligand-target complex activity (e.g., the physical, chemical, energetic, catalytic or thermal state of the ligand, target or ligand-target complex) into a qualitatively or quantitatively different form wherein the conversion produces useful work or a detectable signal. Coupling between the binding of ligand to target and the transducer can be accomplished, e.g., by the transfer of mass, energy, electrons or photons or by coupled chemical or enzymatic reactions that share a common intermediate, mediator or shuttle species. Transducers of the instant invention are components of sensors used to convert the specific binding of a ligand to its target into a detectable signal. Transduction methods include, without limitation, electrical, electromagnetic, electrochemical, optical, piezoelectric, acoustic and thermal detection.

The term “tunable,” when applied to a ligand, means that the conformation of the ligand can be modulated from one analytically or functionally defined state to another in a controlled, operator-, instrument- or device-defined manner by varying the physical or chemical environment of the ligand. Examples of environmental effectors of conformation include temperature, pH, electromagnetic fields (such as electrical fields and magnetic fields), ion concentrations and the concentrations of small molecule effectors. Small molecule effectors include alcohols and DMSO which, by virtue of lowering water activity, will favor transitions toward conformations that result in the net release of thermodynamically “bound” water molecules. Other small molecule effectors include molecules or ions that bind specifically to particular conformations and thereby favor transitions toward those conformations. Examples of such molecules or ions include drugs such as netropsin that bind in the grooves of DNA and intercalators such as ethidium bromide that bind between neighboring base-pairs of duplex DNA. Environmental effectors that modulate the distribution of a tunable ligand among conformational states that differ in target binding affinity will, as a consequence, modulate the affinity of ligand-target binding.

The terms “tunable affinity ligand” and “TAL,” which are synonymous with the terms “multistate affinity ligand” and “conformationally tunable multistate affinity ligand” as used herein, mean a nucleotide-containing ligand that is conformationally tunable through operator-, instrument- or device-defined changes in environmental conditions that yield different conformations of the ligand that are analytically distinguishable from one another and have different affinities for a given target substance. Advantageously, tunable affinity ligands are nucleotide-containing polymers having at least one sequence of nucleotides that participate in intramolecular base pairing to form at least one duplex, triplex, tetraplex, junction, quadruplex or higher order structure under one or more environmental conditions wherein the nucleotide sequence optionally contains a normucleotide spacer or linker group. Essentially, a tunable affinity ligand can exist in at least two different conformational states and can be reversibly changed from one conformational state to another through a defined change in the environment to which the ligand is exposed. The different conformational states can be characterized analytically and/or functionally based, e.g., on spectral signatures, biophysical properties, binding properties and biological activity using methods such as spectroscopic techniques, separation techniques, ligand binding assays, cell-based assays and the like, advantageously including UV spectroscopy, NMR spectroscopy, calorimetry, CD and other methodologies capable of resolving changes in multiparameter distribution of the atoms comprising the tunable affinity ligand under different conditions even in the absence of its cognate target. Thus, the change in affine conformation of the tunable affinity ligand with changes in environmental conditions can be shown to be a property of the ligand itself independent of any conformational change that results from interaction of the ligand with its target.

A tunable affinity ligand can exist in a reversibly switchable plurality of conformational states under different operator-, instrument- or device-defined environmental conditions, where a conformational state is defined as the three-dimensional arrangement of atoms within the ligand with respect to each other. Although different affine conformations of a ligand will typically have different binding affinities for target entities, conditions can sometimes be found where different conformations may have the same binding affinity. For example, two different conformations may have different salt dependences on binding affinity, and one or more uniquely defined salt concentrations might therefore be found where both conformations give the same binding affinity. Conformational states may be characterized and defined by chemical or spectroscopic methods that are sensitive to the relative positions of atoms within the. These conformationally tunable affinity ligands are switchable between:

i) at least a first affinity state corresponding to a first affine conformation of atoms and

ii) at least a second affinity state corresponding to a second affine conformation of atoms,

the affinity of said first affinity state preferably being different in strength or specificity from said second affinity state wherein at least a portion of atoms comprising said first affine conformation also comprises at least a portion of atoms comprising said second affine conformation. Tunable affinity ligands are designed to partition between or among two or more affine states. An affine state of a tunable affinity ligand is a distinct spatio-temporal conformational state that can be defined analytically, such as by spectroscopic, physical, chemical or other experimental means, and is further characterized under a particular set of environmental conditions by a measurable affinity of the ligand for one or more target molecules. The concept of a tunable affinity ligand is distinct from the concept of an affinity ligand with environment-dependent properties, as the target-binding properties of any affinity ligand depend in some way on environmental conditions (e.g., pH, buffer type, salt concentrations and ionic composition). An affinity ligand with environment-dependent properties would include ligands with a single experimentally distinct conformation whose affinity could be altered by changes in environmental conditions and, as such, would comprise essentially all known ligands. In contrast, a tunable affinity ligand is a ligand having at least two distinct affine conformations that can be reversibly interconverted by operator-dependent changes in environmental conditions and that show distinct binding properties to a given target, including differences in magnitude and differences in dependence on environmental. Tunable affinity ligands of the present invention are designed, selected and developed to have:

i) a plurality of at least two controlled, reversible conformational states;

ii) measurable binding to the target substance in one or more of those conformational states;

iii) conformational transitions that occur under conditions that are nonperturbing to the target substance; and

iv) preferential binding to the target substance in at least one conformational state that has lower and differential binding to nontarget substances, such as contaminants present in the sample or separation mixture containing the target substance. This combination of requirements and features distinguishes tunable affinity ligands capable of existing in multiple, environment-dependent states from other ligands, including those selected by fishing from extremely large pools of molecules.

### DETAILED DESCRIPTION OF THE INVENTION

The present invention provides nucleotide-containing tunable affinity ligand-based molecules, complexes, media, kits and devices, including soluble, insolubilized and immobilized constructs, and methods for making and using these compositions, e.g., for preparative, analytical and purification purposes. Applications, include, e.g., molecular and cellular sorting, separations, profiling, detection, diagnostics, discovery, production, processing and quality control. One unique feature of TAL technology as applied to separations is that it enables operator-controlled switching between (analytically and functionally defined) conformations of the ligand rather, as is the case with conventional chromatography methods, than simply changing the interaction of a ligand with its target through nonspecific effects resulting, e.g., from salt gradients that arise when elution conditions are changed. The same principle applies to use of this technology for molecular and cellular detection using self-reporting TALs with affinity transitions designed to interrogate the target surfaces without perturbing the structure or function of the target substance.

Whereas the affinity of ligands used in conventional affinity separations and specific binding assays can be modified by reaction conditions, these changes in affinity are accompanied by nonspecific and/or undefined changes in the target as well as the ligand. The affinity of a therapeutic protein for its target receptor, for example, can be modified by the pH of the reaction mixture. However, both the protein and the cognate receptor are subject to perturbations in structure and stability under affinity-altering conditions,

There is a need in the art for conformationally tunable ligands that switch between well-defined affinity states for a particular target substance to allow ligand binding and release under conditions in which the target substance can be detected, quantified, separated and/or analyzed under conditions in which the target remains structurally and functionally unperturbed.

Disclosed herein are TALs that address this need, thereby providing a diverse array of compositions, methods, kits and systems for highly sensitive, specific, precise and reproducible separation, sorting, detection, profiling, analysis and characterization of target substances under conditions designed to preserve the structural and functional integrity of the target substance. In one embodiment, “nondenaturing TALs” are designed for the separation and detection of relatively fragile targets (e.g., proteins, glycoproteins, lipids, lipoproteins, cell surface antigens and cells) under sufficiently gentle conditions to preserve the structural and/or functional properties of the target not only during and immediately after TAL binding and release, but also for prolonged periods of time, an extremely rigorous test of the structural and functional integrity of the target following TAL-based separation and/or detection, The invention provides for design, preparation and use of rationally designed TALs for the separation, purification, production, processing, detection, quantification and qualification of naturally occurring and synthetic substances, materials and products for research, discovery, development, manufacturing and industrial applications. TAL compositions are described, along with methods, devices, kits and systems for TAL-based applications in detection, separation and analysis of target biological and nonbiological targets. Biological targets include, for example and without limitation, drugs, hormones, transmitters, metabolites, proteins, macromolecular complexes, microorganisms, organelles, prokaryotic and eukaryotic cells and viruses. Nonbiological targets include, for example and without limitation, pesticides and other environmental pollutants, fine chemicals, industrial polymers and chemical warfare agents.

Importance of Molecular Recognition for the Detection and Separation of Target Substances Such as Molecules, Molecular Complexes, Microbes and Cells

The discovery, development and validation of molecular and cellular participants and pathways in health and disease depend critically on the ability to detect molecular interactions with high affinity and specificity. Effective isolation of target substances (e.g., molecules, molecular and supramolecular complexes, microbes and cells) from complex mixtures, cells, tissues, organs and organisms is critically important to accurate discrimination of structure-function relationships and use of well-characterized molecules in biomedical research, diagnostics and therapeutics. Equally important is the ability to detect and quantify target molecules in situ, in vivo and/or in vivo, depending on the particular application.

Naturally occurring and synthetic ligands have been widely used in molecular and cellular separations and detection. Immobilized haptens and antigens, for example, are commonly used as affinity ligands for the chromatographic separation of immunoglobulins from culture media, animal sera, ascites fluid and crude fractions of antibody preparations obtained, e.g., by salt precipitation and gel filtration of these sources. Small molecule drugs and congeners are used as ligands for the isolation and characterization of biological receptors. Conversely, immobilized receptors, cells and membrane fractions are used to isolate and characterize natural and synthetic pharmacophores of biological interest. These same haptens, antigens, ligands and receptors are used in a broad assortment of specific binding assays designed to detect, quantify and characterize target molecules, substances and cells with a high degree of specificity, sensitivity and reproducibility.

The utility of separation and detection methods applies not only to biomedical research and development, but more broadly to life science and industrial applications ranging from environmental and agricultural diagnostics to production, processing, packaging and quality control of foods, chemicals, bulk materials and consumer goods.

Separation science relies heavily on precise and accurate methods for the detection and quantification of substances of interest, i.e., “target substances.” Without target quantification, there is no practical way to determine the effectiveness or efficiency of the separation process. Conversely, detection and quantification of a substance in a complex mixture demands that this substance, the “analyte,” be resolved from other constituents in the mixture, a process that requires either physical, functional, spectral and/or energetic partitioning of the analyte from nonanalyte species. Ultimately, validation of the accuracy with which the analyte is quantified requires isolation, purification and analytical characterization. In this way, the detection and separation of substances in complex mixtures are intrinsically complementary processes.

The present invention relates to rationally designed and empirically selected molecular and multimolecular constructs whose structural and functional properties can be “tuned” in a user-defined manner to achieve desirable performance specifications in a wide array of separation and detection applications. Tunability is imparted by design and synthesis of polymers comprising monomers, dimers or oligomers, linkers, spacers, bridges and shape/charge modifiers strategically positioned to favor intramolecular communication and environmentally responsive structural and conformational rearrangements. Resulting transitions in thermodynamic and kinetic properties of these constructs in response to operator-induced changes in environmental conditions can be applied to sensitive and specific analysis of the surface features of target molecules in their native dynamic states. We refer to these constructs as “tunable affinity ligands” (TALs), because they can be tuned to undergo conformational transitions with mild changes in environmental conditions, allowing dynamic analysis of molecular surfaces and shapes without perturbing the native state of molecular, microbial and cellular targets.

The analytical and preparative capabilities of TALs in molecular and cellular detection, quantification and separation advantageously capitalize on designed conformational diversity that allows stimulus responsive switching between or among conformational states. Importantly, not only can TALs can be designed to undergo intramolecular phase transitions in response to target binding, they can alternatively be designed to undergo conformational transitions that anticipate or trigger target binding. In other words, the functional properties of a particular TAL in binding to or interacting with one or more surfaces of a target molecule, substance or cell depend in a predictable way on the conformational state of the TAL, which conformational state can be designed into the structure of the TAL and controlled by the composition of the medium in which the TAL resides. In fact, a plurality of conformational states can be designed into a given TAL, and the operative state of the TAL can be selected and/or switched among plausible conformations in a rational and reliable manner by simply modifying prevailing conditions, e.g., the solvent or solute composition, temperature or pressure of the surrounding medium or the energies to which the TALs are exposed, e.g., electrical, magnetic, electromagnetic, thermal, mechanical, acoustic or electrochemical energy.

The ability to rationally create multistate molecules with designed control over conformational state as a function of environmental conditions provides a different framework for molecular analysis than conventional heterogeneous and homogeneous binding techniques. In heterogeneous binding techniques, ligand-receptor (or probe-target) binding is typically followed by separation and wash steps that separate bound complexes from solution-phase ligands and/or receptors. In homogeneous assays, e.g., enzyme-modulated immunoassay technology, the activity of a ligand-modified label used to report binding is modulated by a binding event, thereby yielding a detectable signal without the need for separation and wash steps.

Well-established specific binding assay methodologies are very effective in determining the presence and/or or amount of target substances with typically good specificity and sensitivity, but typically provide little information as to the conformational or functional state of the target substance. Antibodies, the most prevalent recognition molecules used in specific binding assays, do not, as a rule, resolve different conformational states of target molecules. Antigens used to immunize animals for the production of antibodies are typically denatured though emulsification and sonication to ensure that the immunized animal's immune system is exposed to all possible binding domains (buried as well as superficial) of the immunizing antigen. Antibody binding to a protein antigen is therefore thought to be essentially independent of the conformation of the amino acid sequence that makes up the binding epitope of the protein. In fact, there is evidence to suggest that the epitope conformation adapts to accommodate the shape/charge distribution of the antibody combining site. Nucleic acid probes bind and detect target sequences with a high degree of sensitivity and specificity and, properly designed, can recognize target sequences in a manner that is independent of the 3-dimensional structure of the target. Ideally, the probe-target binding energy is sufficiently high to disrupt intramolecular base-pairing of the target sequence, thereby altering the conformation of the target sequence. A special type of nucleic acid probe referred to as a “molecular beacon” is designed as a hairpin-forming molecular switch whose loop contains a probe sequence. The intramolecular base-pairing of the stem region predisposes the hairpin to the “closed” state of the switch unless and until target sequences are present, whereupon probe-target hybridization causes linearization of the hairpin structure. In each of these cases, the binding of antibody to antigen or nucleic acid probe to target is reasonably permissive with respect to the pre-bound conformational state of the target. Conversely, the target molecule is subject to a change in conformational state on binding and a change in functional state for those target molecules whose function is conformation dependent, e.g., allosteric enzymes, hormone-coupled receptors, signaling proteins and the like.

The affinity of an antibody for its target depends on the shape-charge distribution of the combining sites of the antibody. The docking surface properties of these antibody-antigen combining sites are substantially maintained by the architectural context of the relatively large protein scaffold on which the recognition sites are displayed. Antibody binding is characterized by an affinity constant (often determined by Scatchard plot) which reflects the association and dissociation rate constants that describe that partitioning of antibody and antigen between free and bound states as a function of antibody and antigen concentrations. Similar principles apply to ligand-receptor interactions well known in the art, e.g., the binding of drugs, hormones and neurotransmitters to receptors, lectins to carbohydrates, biotin to avidin and the like.

The binding strength of a nucleic acid probe for its target is described by the melting temperature at which double-stranded hybrids are denatured into single strands. Below the melting temperature, stable hybrids form (under suitable binding conditions). Above the melting temperature, single strandedness prevails. The melting temperature of a nucleic molecule is substantially determined by the number of nucleotides participating in complementary base pairing (i.e., the sequence length) and the number of participating G-C based pairs (i.e., the GC content), as the binding strength of G-C base pairs is significantly greater than that of A-T base pairs.

Advantageously, TALs designed to undergo environmentally and/or energetically responsive conformational transitions can be triggered in a controlled manner to adopt different quasistable states, each with a different spectrum of exposed surfaces that can interact with the natural diversity of regions, surfaces and groups displayed on a target molecule, cell or substance. The modulatable structural expression of multiple-state TALs endows them with the distinct functionality of comprehensively interrogating different surfaces comprising the native state of a target molecule, substance or cell with far greater selectivity than can be achieved with prior art ligands such as antibodies, lectins and nucleic acid probes.

Discovery, Biophysical Characterization and Optimization of TALs for Chromatographic Separations

TALs are defined sequence polymeric ligands designed, screened and optimized for the affinity separation, detection and identification of target proteins, biomolecular complexes, viruses and cells. TALs are rationally designed such that they change conformation in response to modest changes in solution conditions, temperature and pH. TAL conformation in turn modulates target binding affinities, with binding and release conditions differing for different targets. TAL selectivity derives not so much from the absolute binding affinity of a particular conformation of the TAL for a particular target, but from the environmentally modulated interplay between target binding and conformational switching. This interplay is tuned and amplified by one or more methods in order to separate and/or differentiate multiple target proteins or higher ordered structures.

A few examples of the types of conformational transitions that TALs can undergo include: i) low pH and multivalent cation stabilization of triplex conformations, ii) ion-selective stabilization of quadruplex structures by certain monovalent cations (e.g. K+) and destabilization by other monovalent cations (e.g. Li+), and iii) stabilization of junction structures by hydrophobic species and by multivalent cations. Under the influence of specific environmental effectors such as selected monovalent and multivalent cations, pH and hydrophobic species, rationally designed TALs partition between structured conformations that can have dramatically different affinities for a particular target substance, such as a drug, hormone, lipid, metabolite, soluble or membrane-bound receptor, microbial surface feature, cell surface antigen or intracellular molecule or complex.

Structural information about TAL conformation can be obtained, e.g., by NMR spectroscopy, UV spectroscopy, CD spectroscopy, calorimetry, hydrodynamic, chromatography and electrophoresis. Affinity can be measured under defined conditions using a variety of techniques for detecting and quantifying molecular interactions, including ligand-receptor binding assays such as filtration assays, immunoassays, polarization assays and the like. Functional information can be obtained, e.g., by binding assays and biological assays, including cell-based assays and in vitro, in vivo and in situ testing and imaging.

Environmental Sensitivity of Nucleotide-Based TALs

TALs are designed using our knowledge and experimental data regarding the rich variety of conformational transformations that occur for natural and synthetic nucleic acids, including synthetic oligonucleotides prepared with backbone modifications, normucleotide bases, nucleotide analogs, abasic regions and various types of spacers, linkers, hinges, bridges and shape/charge modifiers. A key feature of these conformational transitions is that they manifest unique sensitivities to solution conditions, ligand interactions and temperature. By engineering TALs to undergo such transitions in a controlled manner, the conformation of TALs can be changed dramatically by modest changes in environmental conditions. It is useful to walk through a few examples of environmentally sensitive nucleic acid transitions and to highlight their consequences for protein binding in order to illustrate some of these concepts.

Examples of environmentally induced nucleic acid conformational changes include the duplex-coil and B-Z transitions of hairpin oligonucleotides and induction of the B-Z transition by binding of the RNA editing enzyme ADAR1. The hairpin to coil transition can be monitored by UV absorbance at 260 nm. At lower temperatures, the hairpin is favored, whereas at higher temperature, the coil form is favored. Temperature-controlled HPLC can also be used to separate hairpin from other forms of DNA (Braunlin et al, 2004). If the hairpin segment contains alternating guanines and cytosines, it has the propensity to form Z-DNA under conditions of high salt or in the presence of multivalent cations. In fact, the B-Z transition of this type of oligomer has been well studied (Benight et al., 1989; Schade et al., 1999). The B form of DNA is the familiar right-handed helical form first described by Watson and Crick (Watson and Crick, 1953), whereas the Z-form is a dramatically different left-handed helical form. Interestingly, the first high-resolution crystal structure of a DNA molecule was a Z-DNA structure of d(CGCGCG) (Wang et al., 1979).

A variety of methods can be used to monitor the B-Z transition, including UV measurements at 295 nm, NMR, CD and affinity chromatography. The CD spectrum provides a useful way to define the B-Z transition, and the temperature-dependence of either the CD spectrum or the UV spectrum can be used to determine the relative fractions of B-DNA hairpin, Z-DNA hairpin and coil.

Z-DNA affinity chromatography has been used to demonstrate that a variety of proteins selectively bind to Z-DNA compared to B-DNA (Fishel et al., 1990). In several cases, a clear biological significance has been ascribed to such interactions (Rich and Zhang, 2003). If a DNA molecule has a propensity for forming Z-DNA, then the binding of such a protein will shift the B-Z equilibrium toward the Z-form. For example, Rich and coworkers have studied the binding of d(CGCGCGTTTTCGCGCG) to the Z-DNA binding protein ADAR1 (Schade et al., 1999). The binding of fragments of ADAR to this oligonucleotide can be monitored by the shift of the CD spectrum from the characteristic B-form to the Z-form.

Under ordinary solution conditions, DNA takes on the so-called B-form conformation. In this right-handed conformation, the sugar conformation is C2′ endo, the base-pairs are nearly perpendicular to the helix axis, and there are clearly defined major and minor grooves. RNA molecules and DNA molecules under conditions of low humidity take on another conformation, the broader and more squat A-form. In the A-form conformation, the sugar conformation is C3′ endo, and the base-pairs are inclined 15° to 20° with respect to the helix axis. In the A-form, the minor groove is wider and shallower and the major groove is deeper and narrower compared to the B-form. Also in contrast to the B-form, the A-form is essentially hollow in the center of the helix. Certain DNA sequences, in particular those with runs of guanine residues, have a natural propensity to take on the A-form (Wahl and Sundaralingam, 1997). For such sequences, the transition toward the A-form is favored by the binding of metal complexes in the major groove (Xu et al., 1995; Xu et al., 1993). By way of example, the addition of Co(NH3)63+ induces A-DNA features for the oligonucleotide d(CCCCGGGG) as can be shown through characteristic changes in CD spectra. A structural characterization this type of transformation can be provided by NMR chemical shifts and NOESY measurements (Xu et al., 1993).

Quadruplex DNA (also referred to as “G-Quartet” or “G-DNA”) is a four-stranded structure that occurs in DNA sequences with strings of two or more neighboring guanines (Burge et al., 2006; Hardin et al., 2000; Shafer and Smirnov, 2000; Simonsson, 2001). Four guanines can form a planar, base-paired tetrameric structure. When runs of guanines occur, stacked tetramers form four-stranded structures that are very stable in the presence of coordinating cations. A variety of unimolecular, bimolecular and tetramolecular quadruplex structures can form depending on prevailing environmental conditions.

Although quadruplex nucleic acid structures were discerned many years ago from fiber patterns of homopolynucleotides, these multi-stranded structures were initially viewed as a curiosity, of no obvious biological relevance. In the years since these initial discoveries, quadruplex DNAs have been implicated in a wide-range of biological processes (Burge et al., 2006; Paeschke et al., 2005; Shafer and Smirnov, 2000; Simonsson, 2001; Van Dyke et al., 2004). Repetitive sequence DNA on the ends of chromosomes (telomeric DNA), G-rich sequences in the immunoglobulin switch region, and the fragile-X repetitive sequence all have a high propensity for forming four-stranded structures in vitro. Quadruplex DNA has also been implicated in the dimerization of HIV RNA and as a control mechanism in various gene-control regions, including the c-MYC oncogene and the Ki-Ras promoter (Cogoi et al., 2004; Fu et al., 1994; Jing et al., 2003; Mori et al., 2004; Siddiqui-Jain et al., 2002; Simonsson et al., 1998). Recently, it has been shown that the intracellular transcription of G-rich regions produces so-called “G-loop” structures, which contain quadruplex DNA on one strand and a stable DNA/RNA hybrid on the other (Duquette et al., 2004).

G-rich oligonucleotide DNAs have pronounced effects on living cells, including antiproliferative activity (Anselmet et al., 2002; Cogoi et al., 2004; Dapic et al., 2003; Dapic et al., 2002; Xu et al., 2001). Such antiproliferative effects may relate to the ability of G-quartet structures to inhibit DNA replication and to induce S-phase cell-cycle arrest (Xu et al., 2001). The existence of such widespread effects suggests specific interactions with key regulatory proteins. It is perhaps not surprising, then, that quadruplex oligonucleotides are often found to bind tightly and specifically to proteins in vitro. Whether or not such interactions have biological significance generally requires additional experimental information. Nevertheless, it is striking that quadruplex nucleic acids interact with key regulatory proteins such as the oncogenic signaling protein Stat-3, the receptor activator of NF-kB (RANK) and the multifunctional nucleolar protein, nucleolin (Hanakahi et al., 1999; Jing et al., 2003; Mori et al., 2004). It is also interesting that the first DNA aptamer whose high-resolution structure was determined turned out to bind to its target, alpha-thrombin, via a four-stranded structure formed from G-rich DNA (Padmanabhan et al., 1993; Schultze et al., 1994). In fact, a variety of both DNA and RNA aptamers appear to bind their target proteins via quadruplex conformations (Dapic et al., 2003). The facts that a) many diverse proteins interact with some specificity with quadruplex DNA, b) quadruplex DNA manifests diverse physiological effects, and c) quadruplex DNA can be destabilized by mild changes in monovalent cation type suggest that nucleotide-containing ligands engineered to have tunable conformations involving synthetic quadruplex-forming oligonucleotides sequences may prove particularly versatile for biotechnological applications such as those described herein.

The requirement of polypurine-rich strands for triplex formation can readily lead to G-rich strands that have a natural tendency to form quadruplex structures. An interesting example of this can be found in the work of Xodo and co-workers (Cogoi et al., 2004) who discovered that triplex-quadruplex equilibria present one potential complication with triple-helix based antisense therapies (Olivas and Maher, 1995). These researchers attempted to regulate expression of the Ki-ras gene in tumor cells by targeting a polypurine/polypyrimidine motif with a G-rich element designed to associate via a triple-helix (Cogoi et al., 2004). The G-rich element did diminish Ki-ras mRNA levels, but apparently by competing for a G-quartet binding protein that bound to the Ki-ras gene region through interaction with a G-quartet structure formed in the purine-rich strand of the control region. It seems likely that discrimination among duplex, triplex and quadruplex structures may play a functional role with certain classes of proteins.

A variety of spectroscopic, thermodynamic and chemical footprinting methods have been used to characterize the formation of quadruplex DNA (Burge et al., 2006), whose CD spectrum shows a characteristic long wavelength maximum at 293 nm in the presence of K+. The addition of methanol has no effect on the CD spectrum, demonstrating little or no effect of methanol on the structure of this quadruplex (though methanol does noticeably enhance its stability). In the UV spectrum, quadruplex formation gives a notable increase in the absorbance at 295 nm, which can be used to monitor the unfolding of the quadruplex at higher temperatures.

Triplex nucleic acids are triple helical structures. Fifty years ago, the formation of nucleic acid triple helices was first reported by Felsenfeld and Rich for synthetic polyribonucleotides (Felsenfeld and Rich, 1957). In the intervening years, the formation of triplex RNA and DNA has provided a rich source for biophysical studies, and numerous structural and environmental factors controlling the thermodynamics and kinetics of triplex formation have been delineated. Sequences with a propensity for forming triplex DNA are widely distributed in eukaryotic genomes (Goni et al., 2006). Recent interest in triple helix formation has been in the context of gene regulation via triple-helix repression of gene control elements.

A nucleic acid triplex can form when a third strand inserts itself in the major groove of a pre-formed duplex and positions itself to make hydrogen-bonding contacts. In order for this to occur for a single nucleic acid molecule, two loop regions are needed, one connecting the Watson-Crick duplex region and another separating the third strand. The thermodynamic behavior of one such molecule, d(GAAGAGGTTTTTCCTCTTCTTTTTCTTCTCC), has been well-characterized by Breslauer and colleagues (Plum et al., 1990). Triple-helix melting curves are characteristically biphasic with the first transition corresponding to dissociation of the third strand and the second to dissociation of the Watson-Crick duplex. Multivalent cations such as Mg²⁺ and spermidine are strongly stabilizing for triplexes. Certain triplexes are also quite sensitive to pH, undergoing dramatic pH-dependent melting.

Triple-helix forming oligomers usually require runs of homopurines and homopyrimidines and can be classified into two basic groups, pyrimidine-purine-pyrimidine (Y•R-Y), and purine-purine-pyrimidine (R•R-Y) (Beal and Dervan, 1991; Beal and Dervan, 1992; Giovannangeli et al., 1992; Griffin and Dervan, 1989; Hoyne et al., 2000; Ono et al., 1991; Semerad and Maher, 1994; Wang and Kool, 1995). Also considered here is a variation of the R•R-Y group where thymine substitutes for adenine in the purine-rich strand ((G,T)•R-Y). In this nomenclature the core duplex is represented by R-Y and is preceded by the third strand, which positions itself in the major groove of the duplex.

Characteristic features of Y•R-Y triplexes are as follows: 1) the third pyrimidine strand sits in the major groove parallel to the duplex purine strand, represented in arrow notation as (↑↑↓); 2) all cytosines in the third strand are protonated; 3) as a consequence of the required protonation, Y•R-Y triplexes that contain cytosines may be quite sensitive to pH; and 4) Such triplexes will also have a fairly high linear charge density and thus will be stabilized by high salt in general and multivalent cations (Mg2+, polyamines, etc.) in particular.

R•R-Y triplexes obey the following rules: 1) the third purine strand sits in the major groove anti-parallel to the duplex purine strand (↓↑↓); 2) thymines can substitute for adenines in the third purine strand (and under some circumstances (see below) this can result in a change in polarity of the third strand); 3) R•R-Y triplexes are stabilized by high salt and multivalent cations (Beal and Dervan, 1991; Beal and Dervan, 1992), though these triplexes are insensitive to pH over a broad range); and 4) A complication with some G-rich triplex forming molecules is that they may have a propensity to form competing quadruplex structures (Olivas and Maher, 1995). (G,T)•R-Y triplexes are a variation of the R•R-Y triplexes where the third strand contains only guanines and thymines. If there are relatively few GpT/TpG steps, the third strand is anti-parallel to the duplex purine strand (↓↑↓). If there are a large number of GpT/TpG steps, then the third strand can assume an orientation parallel to the duplex purine strand (↑↑↓). (G,T)•R-Y triplexes are stabilized by multivalent cations, but are relatively insensitive to pH.

TAL Synthesis

TALs are synthetic polymers, typically polyanionic heteropolymers that can be prepared using a wide variety of solution-phase and solid phase chemistries well-known in the industrial polymer and biopolymer fields. For convenience, the same solid-phase chemistries used for the chemical synthesis of oligonucleotides can be used to produce TALs, including the incorporation of canonical nucleotide monomers as biophysical recognition and conformational control elements. Without prejudice regarding biological compatibility or relevance and limited only by compatibility with solid phase synthesis and post-synthesis conjugation methods, a wide array of functional monomeric elements can be incorporated into TAL sequences using well-established solid-phase chemistries. Solution phase chemistries can also be used with careful consideration to trade-offs of purity, yield, reproducibility and cost.

In the construction of TALs by solid phase synthesis, natural or nonnatural nucleotide bases can be attached to a variety of nonnatural and/or modified backbones (e.g. thioester, polypeptide, morpholino, phosphoramidate and the like). Nonnatural bases with a variety of designed chemical functionalities can be attached to either natural or nonnatural backbones. Synthetic polymer chains comprising, e.g., alkyl glycols or hydrocarbon repeat units, can be inserted between polynucleotide regions in order to provide flexible linkers with desired chemical properties. Reactive chemistries can be incorporated to facilitate conjugation of a variety of functional groups including, but not limited to, amino acids, oligopeptides and a variety of synthetic polymers. Solid phase synthesis can be utilized to incorporate oligonucleotide regions that are exact mirror images (Spiegelmers) of normal oligonucleotides (Vater and Klussmann, 2003). In contrast to the situation in biological systems where a fundamental feature of nucleic acids is their negative charge, TALs can be designed with regions that are neutral, zwitterionic, or even positively charged.

Because they are synthetically constructed, there is no requirement that TALs be compatible with enzymatic methods of oligonucleotide synthesis such as PCR. TALs may be considered a subset of a class of defined-sequence, biomimetic, chain molecules known as foldamers (Hill et al., 2001). Foldamers may obtain complex three-dimensional shapes and thereby interact with exquisite multivalent selectivity to biological target molecules.

TAL Design

TALs are classified according to their conformational behavior and biophysical properties and screened systematically as potential ligands for interacting with and reporting on biological targets. TALs can be designed and optimized to selectively bind to target substances and/or to manifest unique and measurable features (e.g. spectral signatures, biophysical properties, biological activity) upon binding to target molecules and/or assemblies. TALs are designed using established and evolving principles of nucleic acid structure in conjunction with novel and useful design, selection and implementation procedures disclosed herein.

For example, in order to design a helical switch from right-handed B-DNA to left-handed Z-DNA, a sequence with alternating purines and pyrimidine bases is required. If the switch is to favor the Z-conformation, then an alternating GC sequence with methylated cytosines might be chosen. If an array of molecules is desired that undergo the B-Z transition over a range of ionic conditions, then an array of molecules with varying GC ratio and/or extent of methylation might be chosen.

Alternatively, for a hairpin-quadruplex switch, the relative stability of the hairpin vs. the quadruplex depends on the hairpin length, GC content and the number of guanines stabilizing the quadruplex form. Inosine substitution for guanosine can also destabilize the quadruplex. For a hairpin to triplex switch, low pH and Mg2+ will favor the triplex form, while higher pH and the absence of divalent cations will favor the hairpin.

Physical Basis of TAL Separations

To one skilled in physical chemical principles, it is well understood that any protein with a sufficiently large, accessible, positively charged region on its surface will, under the appropriate ionic conditions, show a significant binding affinity for polyanions in general and nucleic acids in particular. The number of proteins with such polyanion binding sites may be larger than previously thought, and these sites may be biologically relevant (Jones et al., 2004). It has been argued that polyanions such as proteoglycans, lipid bilayer surfaces, microtubules, microfilaments and polynucleotides may provide an organizing network for loosely associated proteins, facilitating protein-protein interactions (Jones et al., 2004). This observation is certainly true in the RNA-protein world, where a variety of nucleoprotein complexes play essential functional roles in nucleic acid metabolism and in protein synthesis (notably, the ribosome). The abundance of proteins with natural polyanion binding sites is further supported by the widespread use of heparin affinity chromatography for protein separation (Fountoulakis and Takacs, 1998; Fountoulakis and Takacs, 2002; Fountoulakis et al., 1998; Jones et al., 2004; Langen et al., 2000; Shefcheck et al., 2003; Ueberle et al., 2002; Utt et al., 2002). Surprisingly, even proteins with relatively acidic pIs often have local regions of positive charge that may bind polyanions (Jones et al., 2004; Shefcheck et al., 2003). Moreover, reflecting biological function, proteins can show exquisite shape selectivity for different classes of polyanions (Braunlin et al., 2004; Jones et al., 2004). Given that the success of heparin affinity chromatography for proteomics applications reflects the prevalence of polyanion binding sites on biologically important classes of proteins and the shape-selectivity of such sites for the different polyanions, then by virtue of their conformational flexibility and sensitivity to environmental conditions negatively charged TALs provide an attractive alternative to heparin for proteomics applications.

The binding affinity of negatively charged TALs to positively charged regions on proteins reflects the biologically relevant interaction of native polyanions with such binding sites. As we have demonstrated in our work, enhanced binding to such sites can be obtained by systematically manipulating TAL sequence and conformation. Moreover, since synthetic nucleic acid chemistry allows for variation of charge as well as other chemical functionalities, the range of protein binding sites that are accessible to tight-binding and/or highly selective TALs can be expanded to include not only positively charged sites, but also neutral, and even negatively charged sites.

As chemical entities, TALs have the inherent capability of associating with target molecules through shape-specific, noncovalent interactions. The free energies dominating such interactions may include electrostatic, hydrophobic, hydrogen-bonding and van der Waals components. Nonetheless, several characteristic and highly useful features distinguish TALs from other well-studied chemical entities. First, as linear chain molecules, TALs are conformationally flexible. Second, as foldamers, and in contrast to typical polymeric chain molecules, TALs have the capacity for taking on a variety of well-defined shapes involving hydrogen bonding, base-stacking, ion coordination and protonation events. Third, the linear sequence of chemical monomers making up a particular TAL may be tightly controlled by the step-wise nature of its chemical synthesis on solid phase supports. Fourth, this linear sequence of monomers defines the conformational potential of any particular TAL. Fifth, as we have discussed above and of profound importance for their utility as ligands, the partitioning of a particular TAL among allowed conformational states may be dramatically and precisely controlled by modest variations in solution conditions and temperature.

For a given set of solution conditions, the effect of this sequence-dependent conformational potential on the binding of a given TAL to a target molecule may be determined by binding measurements. Herein we outline an approach to manipulating and optimizing this potential to obtain useful ligands for separation, purification, molecular diagnostics, nanotechnology and drug discovery applications. In one embodiment of this approach, we can take as a starting point known oligonucleotide ligands for particular targets and optimize these ligands for desired binding and release characteristics. In another embodiment, we can start de novo and screen a small database of conformationally diverse TALs (e.g., a library comprising about five up to about one hundred or more oligonucleotides) for binding and release, and then optimize the initial hits from this screen for the desired binding behavior, where optimization includes the ability to synthesize, screen and select TALs from second- and third-generation libraries based on sequence-structure-activity relationships gleaned from the initial library. In another embodiment, we can screen larger, more conformationally diverse libraries from which to cull sequence-structure-activity relationships for the design and selection of focused libraries that zero in on particular regions of sequence-structure-activity space. In any of these embodiments, the guiding design principles derive from correlating biophysical properties (e.g., structure) and behavior (e.g., condition-dependent changes in conformational state) with binding activity. By allowing biophysical behavior to guide design, we dramatically reduce the number of unique TALs that must be examined in order to arrive at molecules with the desired binding and release characteristics. Since our approach does not require enzymatic amplification of oligonucleotide templates, we can incorporate in our design, from the beginning, modified bases, backbones, branch-points and any other chemical entities that are compatible with preferred synthetic methods such as step-wise, solid-phase synthesis and post-synthetic conjugation procedures.

For chromatographic applications, multiple weak interactions along the column may be modulated by shifting TAL conformational equilibria by using mild changes in solution conditions. The resultant modulation in binding affinity to different targets thereby results in high resolution separations. In general, modest differences in intrinsic affinity of two or more closely related targets to the TAL column may be magnified by the optimization of appropriate elution conditions.

Optimization of TALs for Separation and Detection of Serum Proteins

The geometry of the published thrombin aptamer bound to alpha-thrombin has been determined by x-ray analysis (Padmanabhan et al., 1993; Schultze et al., 1994). This molecule forms a G-quartet that spans two positively charged regions on neighboring thrombin molecules. One region is the heparin binding site, and the other is the fibrinogen exosite.

We have demonstrated that when this thrombin aptamer is attached to Sepharose beads, the resultant affinity column binds alpha-thrombin under conditions favoring G-quartet formation (presence of potassium ion) and releases alpha-thrombin under conditions disfavoring G-quartet formation (presence of lithium ion). We also found with the published thrombin aptamer that beta- and gamma-thrombin are well resolved from alpha-thrombin, but are not resolved from each other.

The utility of TAL columns for protein separation depends on what type of separation is desired. As we discuss below, a particular TAL column may give the tightest possible binding (longest retention time) for one specific protein of interest, while another may give the highest resolution separation of the protein of interest from all other proteins. The choice of which column is preferable depends on the desired application.

We have found that 1) replacing a TGT loop in the published aptamer with a TTT loop results in a TAL that shows only a modest change in the retention time of alpha-thrombin (13.2 min vs. 13.0 minutes) but shows clearly enhanced resolution of alpha-thrombin from the overlapping beta- and gamma-thrombin peaks (for the TGT-aptamer, beta- and gamma-thrombin elute at 7.8 min, whereas for the TTT-aptamer, they elute at 7.0 minutes); 2) for the TTT-aptamer, tight binding is maintained for all thrombin variants; 3) superior resolution is obtained for an optimized TAL for which two guanines were replaced by inosines (this replacement destabilizes the quadruplex and thereby shifts the equilibrium away from the form that specifically associates with thrombin and its derivatives); and 4) the elution time for alpha-thrombin decreases to 9.0 minutes for this inosine-variant anti-thrombin TAL compared to an elution time of 13.2 for the TTT-aptamer. The enhanced resolution for the anti-thrombin TAL results primarily from the decrease in elution time for beta- and gamma-thrombin, both of which elute at about two minutes, just after the peak from the void volume. Thus, shifting the equilibrium away from the active (binding) form using rationally designed TALs can significantly enhance the chromatographic resolution.

Though this result may seem counterintuitive, it is in perfect agreement with a simple theoretical model for binding to a TAL that can take on one of two distinct conformations, only one of which binds specifically to the protein of interest. According to this model, binding discrimination can be obtained either by optimizing the specific binding constant K3 compared to the nonspecific binding constant K1 or by destabilizing the tightly bound form of the oligonucleotide by lowering the equilibrium constant K2, which governs the oligonucleotide conformational equilibrium.

The predictions of this model agree well with our results for the thrombin aptamer compared with the inosine-variant TAL. The enhanced separation of alpha-thrombin using the inosine-variant anti-thrombin TAL confirms that destabilizing the high-affinity conformation can be useful for affinity purification applications. In contrast, the behavior of the TTT aptamer, which forms a very stable quadruplex, suggests that stabilizing the high affinity conformation of a TAL is a more effective approach for simultaneously separating a series of closely related proteins (e.g., for proteomics applications). We have also demonstrated the ability to design TALs that bind not only thrombin derivatives, but also other heparin-binding proteins found in serum. These TALs represent attractive candidates for the development of tunable heparin mimetics for proteome pre-sorting applications.

Selectivity and Affinity of TALs Compared to Nontunable Ligands

It has been argued that the more tightly ligands bind to their targets, the more likely they are to bind selectively to them (Eaton et al., 1995). However, the argument presented applies to a very limited selection of ligands and is not generally applicable. In fact, selection for high binding affinity to one target protein, for example, may well result in a ligand that will also bind tightly to closely related proteins. An extreme case of this effect is the RANK aptamer (Mori et al., 2004). Though selected specifically for binding to RANK, when tested for specificity it showed a general affinity for receptors in the TNF family and, in fact, showed 1000-fold higher affinity for the closely related CD30 protein than for human RANK (Mori et al., 2004).

From a theoretical perspective, the conclusions of Eaton et al. have limited applicability (Bonnet et al., 1999; Demidov and Frank-Kamenetskii, 2004; Lomakin and Frank-Kamenetskii, 1998). Of particular relevance for our chromatographic work, according to the theoretical analysis of Bonnet and colleagues, the presence of different conformations of bound and unbound ligand can lead to a simultaneous reduction of binding affinity and enhancement of selectivity (Bonnet et al., 1999).

High affinity ligands have additional problems when used for chromatographic separations. For example, conditions for IgG antibody release from Protein A necessitate partial denaturation and refolding of target IgG. This procedure can lead to a significant reduction in antibody yield and binding activity, compromised quality control and even failure to clear the antibody for use in research, development, manufacturing, marketing and/or sale.

In our chromatographic work, quadruplex TALs based on the published thrombin aptamer bind not only alpha-thrombin, but also beta-thrombin and gamma-thrombin. As we have demonstrated in this example, balancing conformational behavior of rationally modified TALs allows us to magnify existing affinity differences in order to enhance chromatographic separations. An outstanding benefit of this approach is the ability to rationally control both binding and release conditions so that harsh solution conditions and target denaturation can be avoided.

The invention is illustrated through the following examples, which illustrate certain aspects of the invention and are not intended to limit the same.

### EXAMPLES

### Example 1

### Triple-Helical TALs as Tunable Ligands for Chromatographic Separation of Immunoglobulin G Antibodies: Effect of Loop Composition on Retention Times

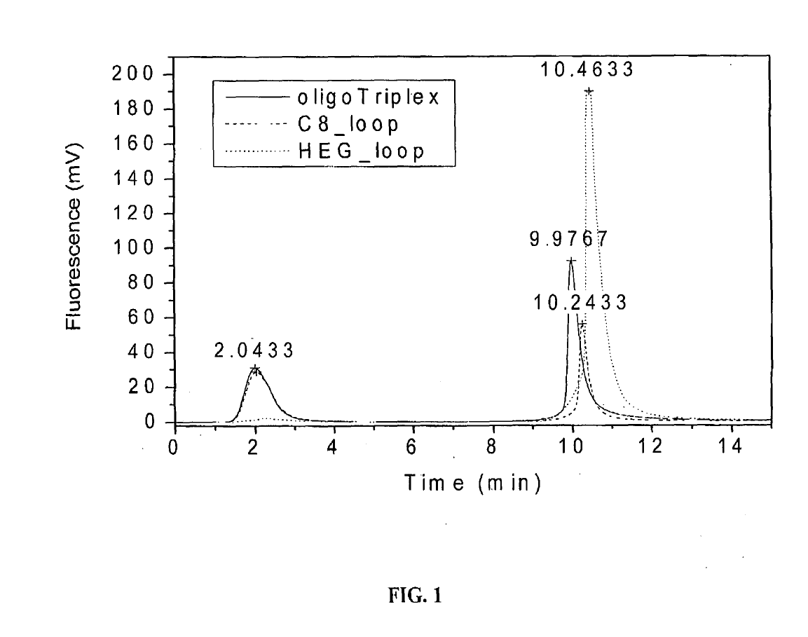

The triple-helix forming TAL, RAD2, was synthesized with an aminohexane linker (C6Am) on the 5′ end to give 5′-C6Am-CCTCTTCTTTTTCTTCTCCTTTTTGGAGAAG-3′. This oligonucleotide was attached to Sepharose beads in a chromatography column using standard coupling chemistries. Briefly, the C6-amino terminal of the oligonucleotide was coupled with n-hydroxysuccinamide moiety of the column. The free NHS activated groups were capped using ethanolamine. For comparison, variants of this TAL containing loop regions with hexaethylene glycol linkers and hexane linkers were also attached to Sepharose beads in a similar manner. These three chromatographic columns were compared for retention efficacy, under gradient elution conditions that were designed to favor the tightly binding conformation at the beginning of the experiment and to favor the weakly binding conformation at the end of the chromatographic elution. Under these gradient conditions, the TAL variant RAD1 with the hexaethylene glycol linkers (CCTCTTC(HEG)CT TCTCC(HEG)GGAGAAG) showed enhanced retention compared to the other variants (see FIG. 1). In this experiment, at time 0, a sample of fluorescein-labeled human IgG (Jackson ImmunoResearch Laboratories, West Grove, Pa.) was injected onto the column, and fluorescence was monitored as a function of time using excitation at 490 nm and emission at 528 nm.

### Example 2

### Triple-Helical TALs as Tunable Ligands for Chromatographic Separation of Immunoglobulin G Antibodies: Separation of IgG from Complex Samples

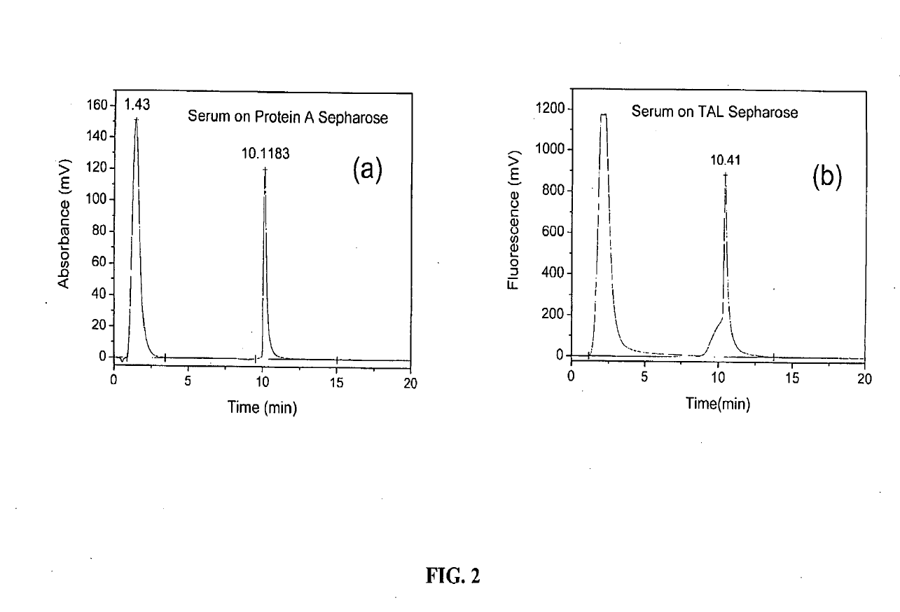

The column prepared from the TAL variant with the hexaethylene glycol linkers, RAD1, CCTCTTC(HEG)CTTCTCC(HEG)GGAGAAG, was further examined for its ability to separate IgG from complex biological samples, and the results were compared to separations of IgG from these samples performed using a Protein A-Sepharose column. The results of such a comparison are shown in FIG. 2, where we compare the separation results for a serum sample run over a Protein A-Sepharose column to those on the RAD1 Sepharose column. The peak at 10.1 minutes collected from the Protein A-Sepharose column and the peak at 10.42 minutes collected from the RAD1 column were each electrophoresed over a 4-12% polyacrylamide gel, using 1×SDS buffer and compared with IgG standards and molecular weight markers. After silver staining, we saw only two bands from each sample, one at about 50 kD and the other about 25 kD, as expected after breaking of all the disulfide linkages. The two bands from the TAL-purified sample corresponded with the two bands from the Protein A-purified sample and with the two bands of the IgG standard. We conclude that the purity of the TAL-purified serum sample is indistinguishable from the purity of the Protein A-purified sample, as judged by SDS gel electrophoresis.

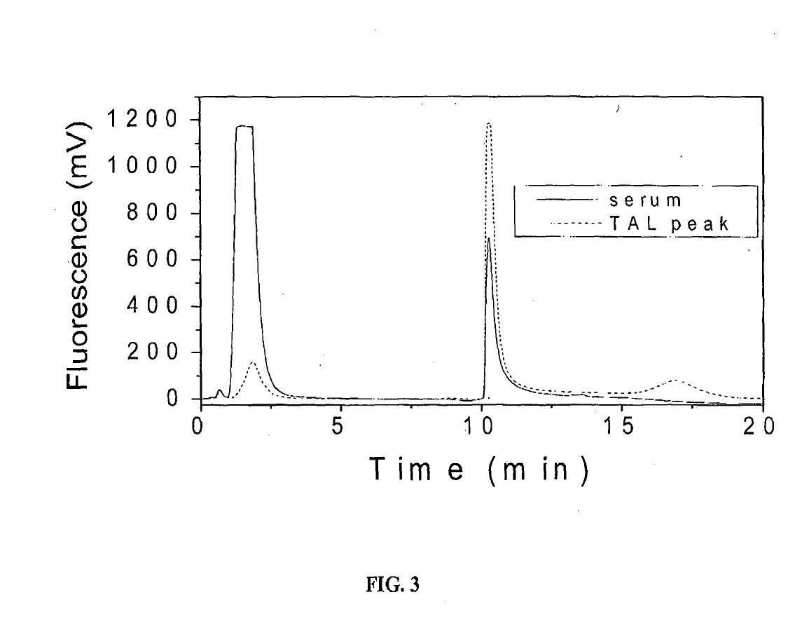

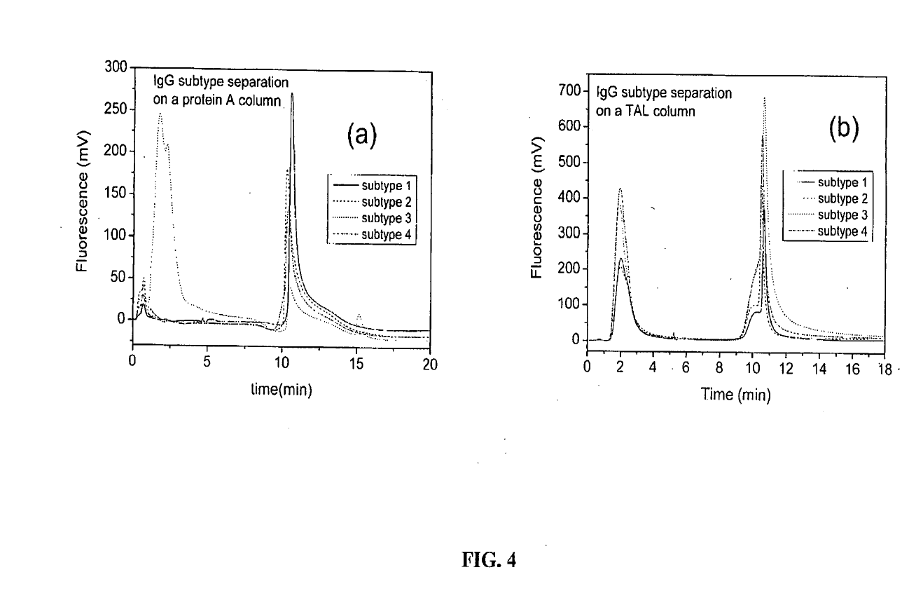

Also, as shown in FIG. 3, when material collected from the peak at 10.42 minutes from the RAD1 column is reinjected onto a Protein A-Sepharose column, most of the peak is retained at the position characteristic of IgG. The small amount of protein that comes through in the void volume appears to correspond to IgG subtype 3 (IgG3). In contrast to the Protein A column, the RAD1 column retains all IgG subtypes with comparable efficacy (see FIG. 4). Indeed, as seen in FIG. 4, the slightly longer retention time that we observe for the IgG3 subtype compared to the other subtypes suggests a modestly higher affinity of the TAL column for IgG3 than for the other subtypes. For applications requiring separation of some or all of the IgG subtypes from one another, a shallower gradient represents a preferred approach to enhance resolution among subtypes.

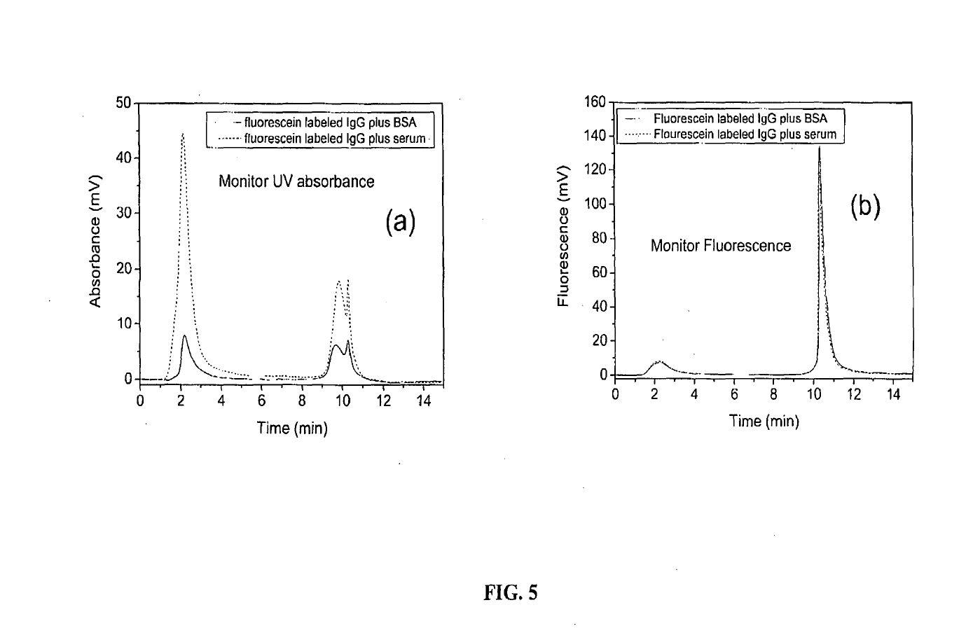

A further demonstration of the ability of the RAD1 column to bind specifically to IgG is shown in FIG. 5, which shows the results of separations of fluorescein labeled IgG from 1) a sample containing labeled IgG plus BSA and 2) a serum sample that was doped with fluorescein-labeled IgG. Interestingly, a comparison of the UV and fluorescence signals of the serum sample (which contains unlabeled IgG from the blood) suggests a partial resolution of labeled and unlabeled IgG, again with the application of a step gradient. This observation suggests that TAL technology can separate closely related proteins differing only in the extent of fluorescent labeling.

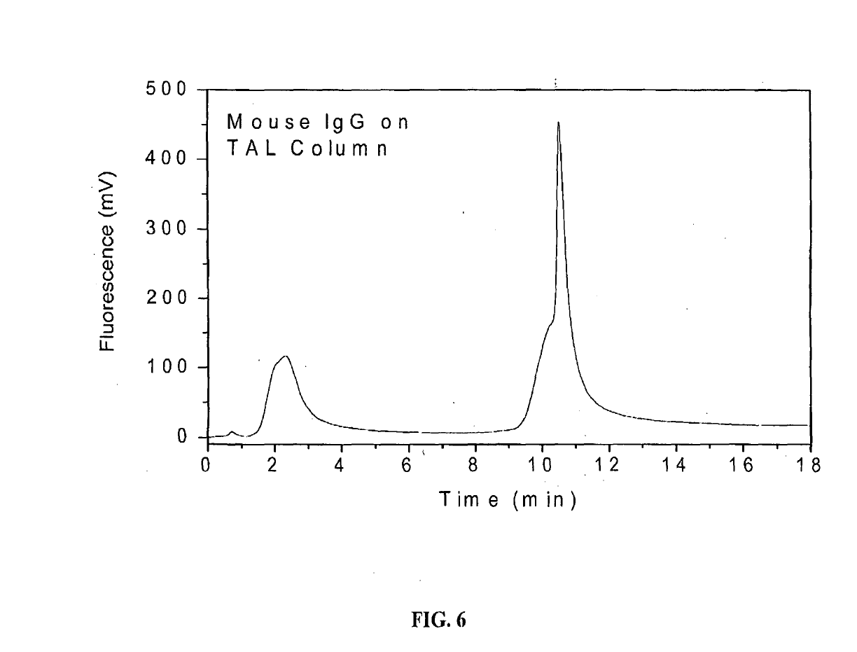

Recently, we have examined the ability of our lead IgG-binding TALs to separate human from mouse IgG. As shown in FIG. 6, the RAD1 column does bind tightly to mouse IgG, as it does to human IgG suggesting that it will be possible to separate human from mouse antibodies through gradient optimization with TAL candidates.

### Example 3

### Quadruplex-Forming TALs for Separation and Detection of Serum Proteins

The published thrombin aptamer bound to alpha-thrombin forms a G-quartet that spans two positively charged regions on neighboring thrombin molecules (the heparin binding site and the fibrinogen exosite) as determined by x-ray analysis (Padmanabhan, Padmanabhan et al., 1993; Schultze, Macaya et al., 1994. When this thrombin aptamer is attached to Sepharose beads, the resultant affinity column binds alpha-thrombin under conditions favoring G-quartet formation (presence of potassium ion) and releases alpha-thrombin under conditions disfavoring G-quartet formation (presence of lithium ion). With the published thrombin aptamer affinity column, beta- and gamma-thrombin are well resolved from alpha-thrombin, but are not resolved from each other.

The utility of TAL columns for protein separation depends on what type of separation is desired. As we discuss below, a particular TAL column may give the tightest possible binding (longest retention time) for one specific protein of interest, while another may give the highest resolution separation of the protein of interest from all other proteins. The choice of which column is preferable depends on the desired application.

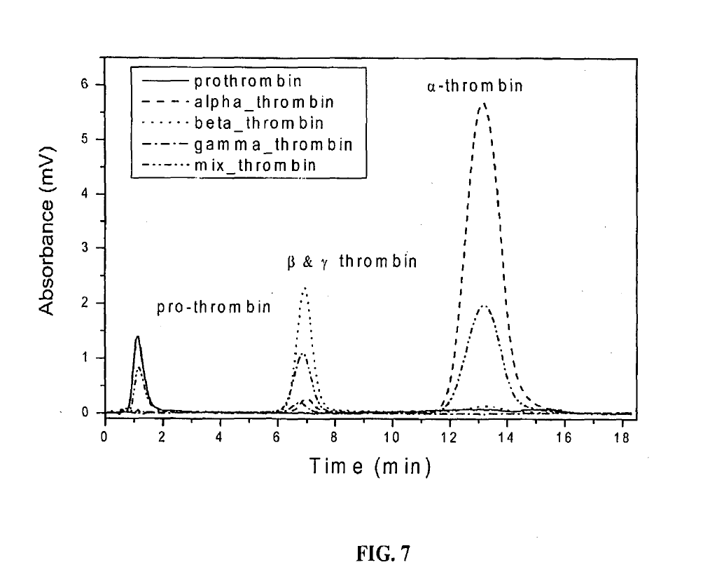

As shown in FIG. 7, replacing a TGT loop in the published aptamer with a TTT loop results in a TAL that shows only a modest change in the retention time of alpha-thrombin (13.2 min vs. 13.0 minutes) but shows clearly enhanced resolution of alpha-thrombin from the overlapping beta- and gamma-thrombin peaks. (for the TGT-aptamer, beta- and gamma-thrombin elute at 7.8 min, whereas for the TTT-aptamer, they elute at 7.0 minutes). Note also that for the TTT-aptamer, tight binding is maintained for, all thrombin variants.

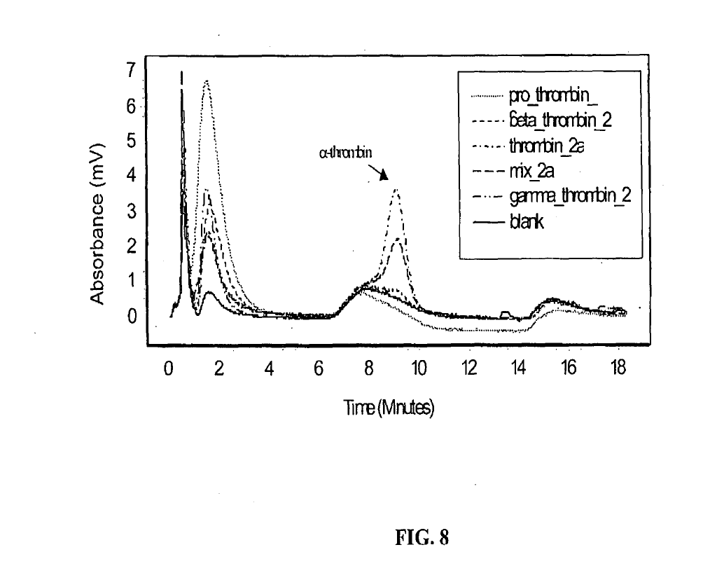

As shown in FIG. 8, even better resolution was obtained for an affinity column prepared from an optimized TAL for which two guanines were replaced by inosines during solid phase synthesis of the oligonucleotide. This replacement destabilizes the quadruplex and thereby shifts the equilibrium away from the form that specifically associates with thrombin and its derivatives. Consistent with this idea is the observation that the elution time for alpha-thrombin decreased to 9.0 minutes for this inosine-variant anti-thrombin TAL compared to an elution time of 13.2 for the TTT-aptamer. The enhanced resolution for the inosine variant anti-thrombin TAL column resulted primarily from the decrease in elution time for beta- and gamma-thrombin, both of which eluted at about two minutes, just after the peak from the void volume. We conclude that shifting the equilibrium away from the active (bound) form can indeed enhance the chromatographic resolution.

The TAL shown in FIG. 8 was further optimized through rational and combinatorial substitutions to provide several variants of nondenaturing TALs. The nondenaturing property of the TALs was demonstrated by analytical experiments indicating that targets released from TAL-target complexes remain structurally and functionally intact. This nondenaturing property is a unique property of TALs that are capable of reversible partitioning between target-bound and free states under the influence of extremely subtle changes in the environmental conditions in detection, separation and sensing applications (including real-time monitoring of the presence and amount of target substance in a sample). For example, after binding or separation experiments using thrombin and other catalytically active and potentially labile proteins, the “post-processing” physical, chemical and enzymatic activities of “detected” or “separated” target can be shown to remain essentially unchanged relative to control (unprocessed or mock-treated) targets that have not been exposed to TALs. The structural and functional integrity of a “detected” or “separated” target is also monitored in real-time and accelerated stability studies using physical, chemical and biological assay techniques capable of detecting even minor changes in the structural features, binding properties, catalytic activities and bioactivity of TAL-treated targets relative to controls.

### Example 4

### Circular Dichroism-Based Demonstration of TAL Conformational Transitions

The thrombin aptamer forms a four-stranded quadruplex DNA structure. As demonstrated by X-ray crystallography, this quadruplex conformation binds selectively to the blood clotting protein thrombin. We used CD to monitor the stabilities and structures of a tunable form of the thrombin aptamer, the inosine-variant anti-thrombin TAL (see Example 3 above and FIG. 8) that undergoes a transition from a quadruplex to a Watson-Crick hairpin form. Using CD to analyze the inosine-variant anti-thrombin TAL, we defined a combination of KCl and ZnSO₄ concentrations that converted the structure from the thrombin-binding quadruplex to the thrombin-nonbinding hairpin. We confirmed previous observations that K⁺ stabilizes the quadruplex form and Zn²⁺ destabilizes the quadruplex form. By making systematic adjustments of both KCl and ZnSO₄ concentrations, it was found that 50 mM Tris-HCl (pH 7) containing 50 mM KCl and 10 mM ZnSO₄ led to 50% dissociation of the quadruplex form at 40° C.

The hairpin-quadruplex tunable ligand (HPL) had the sequence ⁵′ CCAAC GGTTGGT3GGTTGG^{3′}. This oligonucleotide was purchased from IDT, who produced it by solid phase synthesis followed by HPLC purification. CD measurements were performed using a Pistar Kinetic Circular Dichroism Spectrometer (Applied Photophysics, Leatherhead, UK). The temperatures were set at a minimum of 20.0° C. and a maximum of 90.0° C. in 10.0° C. increments with the solution stabilizing at each temperature for 10 minutes before data extraction. The bandwidth was set at 1.0, the time per point at 1.0000, and the step at 0.5. The minimum wavelength was set at 200 nm and the maximum at 350 nm. The data was set to repeat 5 times per temperature. A quartz cylindrical CD cell was used (Hellma model 121.00 (QS), pathlength 5 mm, sample volume 850 μl). This CD cell was cleaned with H₂O, then acetone and allowed to air dry. Blank data was used as the baseline and subtracted from each data set. The data was plotted versus temperature for each molecule or ionic condition. Quadruplex formation was monitored by ellipticity at 290 nm, while the ellipticity at 242 nm was sensitive to both hairpin and quadruplex formation.

### Example 5

### Destabilizing Active TAL Conformations can Enhance Binding Specificity, while Reducing Overall Binding Affinity

The results of Example 3 agreed with a theoretical model for binding to a TAL that can take on one of two distinct conformations, only one of which binds specifically to the protein of interest. As discussed above, binding discrimination can be obtained either by optimizing the specific binding constant K₃ compared to the nonspecific binding constant K₁ or by lowering the equilibrium constant K₂, thereby shifting the oligonucleotide conformational equilibrium. The outlines of the model are as follows:

Variants of the thrombin-binding TAL can exist either as a relatively poorly structured coil form or as a highly structured quadruplex. The equilibrium between coil and quadruplex will depend on the type and concentrations of monovalent cations. Here, we will restrict ourselves to a situation where only two types of monovalent cation are present, potassium and lithium. Potassium binding is required to stabilize the quadruplex, whereas Li⁺ destabilizes the quadruplex. The system is governed by the following equilibria:

D+ [symbol] DP+mM⁺ D+nK⁺ [symbol] D*+pM⁺ D*+P [symbol] D*P+qM⁺

where D is the TAL in the coil form, D* is the TAL in the quadruplex form, P is the protein target, DP is the nonspecific TAL-protein complex, and D*P is the quadruplex-protein complex. M⁺ is monovalent cation (in this instance, either Li⁺ or K⁺), and m, n, p, and q represent the cation stoichiometries of the various ion-exchange reactions. The above equilibria are governed by the equilibrium expressions:

$$\begin{matrix}{K}_{1}^{T} = {{K}_{1}^{obs} ( {M}^{+} )}^{m} & {K}_{2}^{T} = {K}_{2}^{obs} \frac{{( {M}^{+} )}^{p}}{{( {K}^{+} )}^{n}} \\ {K}_{3}^{T} = {{K}_{3}^{obs} ( {M}^{+} )}^{q} & \end{matrix}$$

$$where {K}_{1}^{obs} = \frac{( DP )}{( D ) ( P )} , {K}_{2}^{obs} = \frac{( {D}^{*} )}{( D )} and {K}_{l}^{obs} = \frac{( {D}^{*} P )}{( {D}^{*} ) ( P )}$$

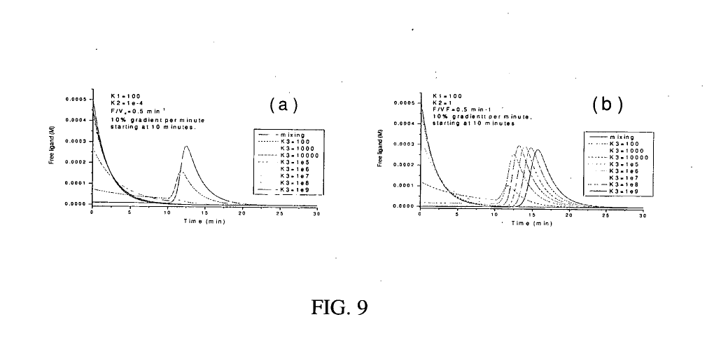

For illustration, we assume that q=m=4, and n=p=3 and simulate a stirred flow reactor (Chen, Chen et al., 1998) into which a sample is injected, which is then followed by a gradient of two buffers. We also assume that the ratio F/V_{c}, of the flow rate to the volume of the flow cell, is equal to 0.5 min⁻¹. The resulting differential equations for ionic conditions are straightforward to solve analytically. The equilibria are solved analytically for free protein concentration at any set of ionic conditions using numerical determination of the mass-balance of protein concentrations. The results of these calculations are shown in FIG. 9.

### Example 6

### TAL and Labeled Hairpin TAL Design Considerations

The predictions of theoretical modeling agree well with our results for the thrombin aptamer compared with the inosine-variant anti-thrombin TAL (see Example 3 above) under target-binding and target-nonbinding conditions. The enhanced separation of alpha-thrombin shown in FIG. 8 for the inosine-variant TAL confirms that destabilizing the high-affinity conformation is a useful strategy for affinity purification applications. In contrast, the behavior of the TTT aptamer, which forms a very stable quadruplex, confirms that a useful method for simultaneously separating a series of closely related proteins (that differ e.g. only in post-translational modifications) is to stabilize the high affinity conformation.

The equilibrium between hairpin and quadruplex depends on the type and concentrations of monovalent cations. Here, we will restrict ourselves to a situation where only two types of monovalent cation are present, potassium and lithium. Potassium binding is required to stabilize the quadruplex, whereas Li⁺ destabilizes the quadruplex. The system is governed by the following equilibria:

D+P [symbol] DP+mM⁺

D+nK⁺ [symbol] D*+pM⁺

D*+P [symbol] D*P+qM⁺

where D is the TAL in the hairpin form, D* is the TAL in the quadruplex form, P the protein target, DP the hairpin-protein complex, and D*P the quadruplex-protein complex. M⁺ is monovalent cation (in this instance, either Li⁺ or K⁺), and m, n, p, and q represent the cation stoichiometries of the various ion-exchange reactions. The above equilibria are governed by the equilibrium expressions:

$${K}_{1}^{T} = {{K}_{1}^{obs} ( {M}^{+} )}^{m} \qquad {K}_{2}^{T} = {K}_{2}^{obs} \frac{{( {M}^{+} )}^{p}}{{( {K}^{+} )}^{n}} \qquad {K}_{3}^{T} = {{K}_{3}^{obs} ( {M}^{+} )}^{q}$$

$$where {K}_{1}^{obs} = \frac{( DP )}{( D ) ( P )} , {K}_{2}^{obs} = \frac{( {D}^{*} )}{( D )} and {K}_{3}^{obs} = \frac{( {D}^{*} P )}{( {D}^{*} ) ( P )}$$

As we discuss in structural terms below, by balancing quadruplex and hairpin structures, a range of QH labeled hairpin TALs are designed with a range of K₂^{T} values. Scaffolds and linkers are varied to mimic genomic G-rich regions, including telomeres, the c-MYC promoter region and fragile X expansion regions.

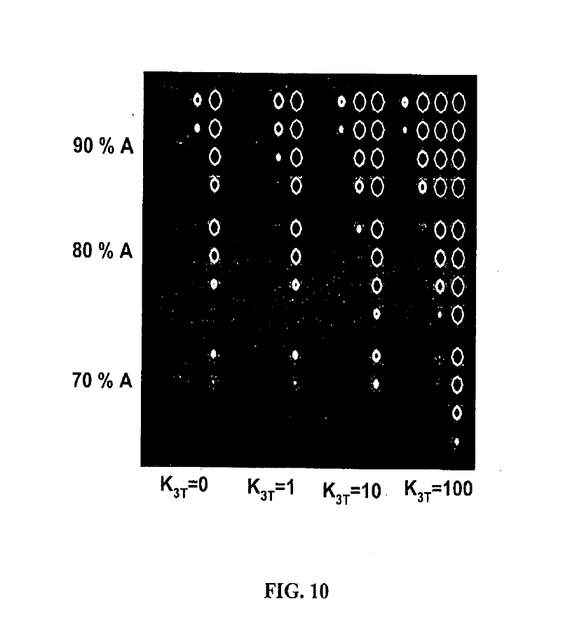

In FIG. 10 we show a simulation illustrating the types of data expected. For the calculations, we assumed that n=p=3 and m=q=4. In this simulation, we show the results for a 4×4 array of labeled hairpin TALs, with K₂^{T} values that increase from left to right and from bottom to top. The actual array values are given in the figure. The x-axis shows increasing K₃^{T} values, whereas the y-axis shows increasing fraction of K⁺-containing buffer as described in the figure legend. It can be discerned from this plot that distinct intensity patterns are observed for proteins based solely on their intrinsic binding affinities for the quadruplex form of the labeled hairpin TAL. Arrays of such labeled hairpin TALs with varying K₃ values for different proteins can be designed to provide additional levels of, discrimination.

Labeled hairpin TAL design requires attention to the stabilities of at least two distinct conformations under the influence of selected reaction conditions. For each individual labeled hairpin TAL, a balance needs to be made between the relative stabilities of, e.g., quadruplex and hairpin forms. As is shown by example in FIG. 10, if the quadruplex form is too stable (e.g., the upper right hand corner of each 4×4 matrix), then the molecule is always in the quadruplex and is not an effective reporter on protein binding. Alternatively, looking at the lower left hand corner of each matrix, it is clear that if the hairpin is too stable, then even the presence of specifically binding proteins may not suffice to switch the labeled hairpin TAL into the fluorescent “on” position.

To confirm hairpin to quadruplex transitions observed by CD (see Example 3 above), we purchased labeled hairpin prepared by solid phase synthesis using 5′ and 3′ donor-acceptor label pairs designed to detect thrombin binding by fluorescence quenching (e.g., acceptor quenching of donor fluorophore emission). Anti-thrombin TALs were labeled with fluorescent donor-quencher pairs that fluoresce only in the target-bound (or target-unbound) state. The transition from duplex to quadruplex forms of the inosine-variant anti-thrombin TAL could be detected by target-dependent switching between high and low target-binding affinity conformations with changes in reaction conditions (see Example 14 below). Unlike commercially available molecular beacons, whose utility is limited to detection of nucleic acid targets, labeled hairpin TALs are well-suited for the detection and monitoring of nonnucleic acid targets. Target recognition by labeled hairpin TALs can be detected by fluorescence energy transfer or fluorescence quenching of donor-acceptor pairs or by a variety of alternative modalities, including direct electrical detection of unlabeled constructs as described below.

In this example, several factors are illustrated for labeled hairpin TAL design. First, environmentally modulated specificity is incorporated by designing families of TALs that switch between hairpin and quadruplex forms under different conditions. Second, in addition to this environmental component of specificity, there will be a recognition component. For example, quadruplexes formed from different sequences will have different loop sequences that will impact recognition of particular proteins. Third, the incorporated dyes may modulate TAL conformation and binding interactions. Fourth, as indicated by our thrombin data, kinetic effects offer another window on specificity.

### Example 7

### TALs with Four-Stranded, pH-Switchable States Involving Cytosine Protonation

TALs such as d(CCCCTTTTCCCCTTTTCCCCTTTTCCCC) are capable of folding back on themselves to form four-stranded structures involving hemiprotonated C-C+base pairs, which intercalate between neighboring C-C+base pairs to form four-stranded i-motif structures. Such structures form at relatively low pH, where protonation is possible, but are disrupted at higher pH, where protonation is disfavored. The unique shape and charge structure of i-motif oligonucleotides provides a useful means of discriminating target proteins, microbes and cells for separation and profiling.

### Example 8

### Multiple-State TALs

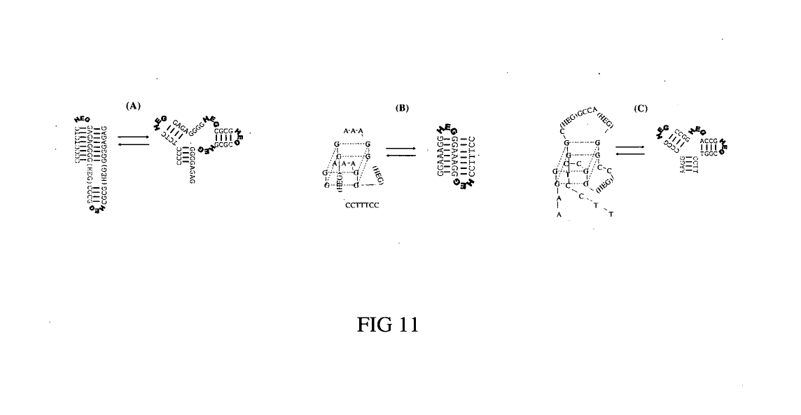

TALs were designed to switch among multiple states in response to environmental stimuli, where “multiple” in this context includes “greater than two states” A few examples of two-state TALs are shown in FIG. 11. In this figure, the triplex conformations may be stabilized by low pH and the presence of multivalent cations. The quadruplex is specifically stabilized by certain monovalent cations (e.g. K⁺) and destabilized by other monovalent cations (e.g. Li⁺), and the junction structure is stabilized by hydrophobic ligands and by multivalent cations.

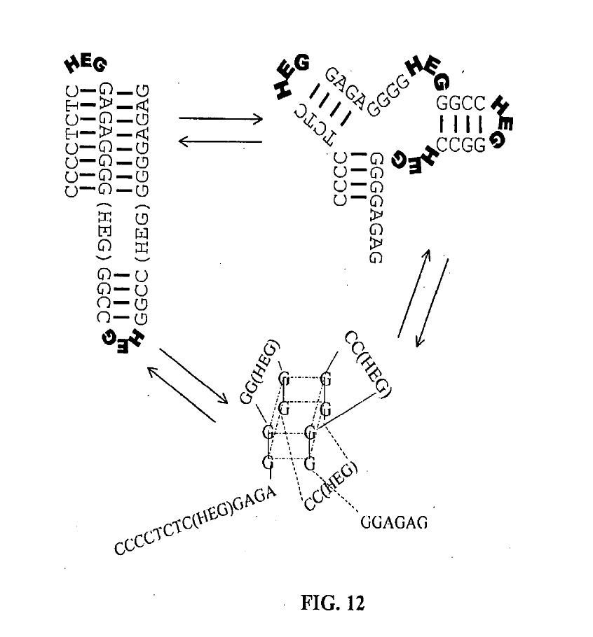

An example of a three-state TAL is shown in FIG. 12. In this figure, the triplex form is stabilized by high salt and Mg²⁺, the three-way junction is stabilized by binding of hydrophobic ligands, and the quadruplex structure is stabilized by monovalent cations such as K⁺.

### Example 9

### TALs for Proteome Sorting

We found that G-quartet forming TALs bind not only thrombin derivatives, but also other heparin-binding proteins found in serum. Based on this result, we predicted that G-quartet forming TALs will prove useful as tunable heparin mimetics for proteome sorting applications. The use of such tunable heparin mimetics with other two-state and higher order multiple-state TALs allows much more refined presorting potential than is possible with heparin or with other chromatographic methods. The physical basis for this sorting is found in the interaction of conformationally flexible TALs with complementary regions on proteins.

### Example 10

### TALs for Sorting Bacteria, Viruses, Viral Fragments and Cells

We have shown that TALs respond dramatically to modest environmental changes under physiological and near-physiological conditions where cell-surface proteins are maintained in their native conformations. Consequently, the interaction of TALs and TAL conjugates with proteins on the surface of viruses or prokaryotic or eukaryotic cells provides a mechanism for a) sorting of viruses, fragments of viruses and cells and b) detection and profiling of viruses, fragments of viruses and cells. For the cell sorting application, TALs are attached to chromatographic media, magnetic beads or other modified surfaces and allowed to interact with the viruses or cells under solution conditions favoring binding. A washing step is used to remove unwanted debris, and viruses or cells are released in order of binding strength using continuous or step gradients that switch the TALs among binding conformations. One application of this method is the purification of inactivated viruses or viral fragments for the production of vaccines. Another application is the separation of progenitor cells from their more differentiated progeny or less differentiated precursor or stem cells.

### Example 11

### TALs for Profiling Bacteria, Viruses, Viral Fragments and Cells

A panel of self-reporting TALs is allowed to interact with the target cells, viruses or viral fragments under solution conditions favoring binding. In a preferred embodiment, the TALs are attached to beads or surfaces. Alternatively, the TALs may be designed with distinguishable spectral properties, allowing them to be used in homogeneous assays. The characteristic spectroscopic response of the TALs with target under variable solution conditions functions as an “electronic tongue” to define the cells, viruses or viral fragments present.

### Example 12

### Methods for Monitoring Protein Integrity

The ability of nondenaturing TALs to bind to and release target proteins in a manner that retains essentially full integrity of the TAL-exposed protein (i.e., essentially no detectable degradation) can be monitored by a variety of functional, structural, chemical and spectroscopic means. For example, CD measurements were used to quantify the fractions of alpha-helix, random coil and beta-sheet within proteins (e.g., clotting proteins, immunoglobulins and their cognate antigens). Fully or partially denatured proteins show a change in these parameters. Most prominently, denatured, partially denatured and/or functionally compromised proteins tend to show an increase in the relative fraction of random coil. Conversely, proteins exposed to nondenaturing TALs for prolonged periods (e.g., up to 12 hours) show no change in the relative distribution of alpha-helix, random coil and beta-sheet structure. NMR measurements also show clearly the effect of protein denaturation. Amino acids in random coil environments show characteristic chemical shifts and enhanced longitudinal relaxation rates compared to amino acids in structured environments, which show a wider range of chemical shifts and generally reduced longitudinal relaxation rates. Functional assays of enzyme activity show enhanced kinetic rates for enzyme activity per mass of protein compared to fully or partially denatured proteins. Partially or fully denatured proteins generally have an increase in solvent exposure of hydrophobic groups. Hydrophobic dyes such as bromphenol blue bind specifically to exposed hydrophobic groups on proteins and provide a good means of spectrophotometrically monitoring protein denaturation among target proteins exposed to denaturing ligands. Conversely, proteins that remain functionally intact following exposure to cognate TALs for periods ranging from minutes to hours (as determined by immunoassay and cell-based assays) show no statistically significant increase in bromphenol blue absorption relative to control, untreated target proteins.

The nonperturbing property of nondenaturing TALs can be further illustrated using real-time and accelerated stability studies of TAL-exposed target proteins vs. untreated controls, antibody-purified proteins and variable buffer-exposed proteins. Even proteins that remain structurally intact immediately following potentially destabilizing conditions (as determined by structural and functional assays described here) are shown to exhibit spectral, binding and activity changes over time in real-time and temperature-accelerated stability studies using the same assay techniques.

### Example 13

### Methods for Monitoring Cell Viability

The ability of nondenaturing TALs to bind to and release cells and other complex biological structures can likewise be monitored by a variety of tried and tested methods. For example, cell viability is monitored by a) mitochondrial function assays, b) apoptosis assays and c) membrane integrity assays. Mitochrondrial function, for example, is monitored by MTT (a tetrazolium dye that is reduced to a colored product in live cells), by oxygen consumption rate measurements and by assaying ATP, which decreases for dead cells compared to viable cells. Apoptosis can be monitored by measurements that are sensitive to caspase activity or to phosphatidylserine externalization. The propidium iodide dye assay is used to measure membrane integrity. Flow cytometry is used to measure the presence and relative distribution of cell surface markers (e.g., CD34, CD45) in cell populations exposed to cognate TALs vs. untreated control cells.

### Example 14

### TAL-Based Thrombin Detection Using Fluorescence Energy Transfer Assays

Confirming the state-dependent affinity of TAL construct for target molecules, we demonstrated that different variants of the thrombin-binding construct can be used to detect thrombin in fluorescence energy transfer assays. Fluorescently labeled TAL constructs designed to undergo transitions from hairpin to quadruplex conformations under the influence of changes in buffer conditions were prepared by solid phase synthesis.

By labeling the 5′ and 3′ ends of spacer-modified oligonucleotides (designed to undergo hairpin to quadruplex transitions) with donor-acceptor label pairs (e.g., Cy3 donor with Dabcyl quencher (Integrated DNA Technologies, Coralville, Iowa), we have shown that G- and T-rich hairpin-forming oligonucleotides can undergo structural transitions from thrombin-nonbinding to thrombin-binding conformations as shown by increasing fluorescence when the ionic composition of the buffer is changed (e.g., from 125 mM TEAA, 10 mM KCl, pH 6.5 to 500 mM LiCl, 10 mM TEAA). Whereas in the TEAA-KCl buffer, the hairpin form of the spacer-modified oligonucleotide is favored, a transition to the quadruplex form occurs in the LiCl-TEAA buffer as shown by CD and confirmed by time-dependent increases in fluorescence of the Cy3/Dabyl-labeled TAL.

### Example 15

### Direct Electrical Detection of Thrombin Using Anti-Thrombin TAL

To show that thrombin binding is dependent on the oligonucleotide conformation rather than nonspecific interactions with donor or acceptor fluorophores, experiments are performed using silicon-based capacitative devices to detect thrombin binding with unlabeled inosine-variant anti-thrombin TALs attached at the 5′ end to self-assembling monolayer-modified silicon substrates. Inosine-variant TALS were prepared and analyzed according to the methods of Example 3. Transitions from the thrombin-nonbinding state in 125 mM TEAA containing 10 mM KCl to the thrombin-binding state in 10 mM TEAA containing 500 mM LiCl are measured by changes in dielectric permittivity and capacitance. Changes in relative capacitance are detected with thrombin-binding to quadruplexes compared with nonsense sequences. Conformational transitions of thrombin-binding TALs are confirmed by melting curves showing distinct phase transitions of the G-rich, TTT-loop oligonucleotides compared to nonsense sequences and by CD showing spectral shifts characteristic of quadruplex formation when conditions are changed from KCl- to LiCl-containing buffers.

The above capacitance-based detection method illustrates a tunable affinity ligand-based sensor that relies on an electrical transducer to measure ligand-target binding to monitor target substances in reaction mixtures. Unlike heterogeneous binding assays that require physical separation of target-bound complexes from unbound ligands, tunable affinity ligand-based sensors can be used to measure both increases and decreases in concentration of target substances as the ligand partitions between target-binding and target-nonbinding states in a reversible manner that depends on the potassium- versus lithium-dependent state of the ligand. Affinity ligands designed for separation or detection of target substances can therefore be screened and selected for environmentally sensitive tunability and validated for target association and dissociation properties with sensor-based methods using label-free electrical detection as an alternative to fluorescence methods that require oligonucleotide labeling and optical filtering, circumventing the need to label oligonucleotides

In designing and testing tunable affinity ligands for target binding and release properties, we note that higher order structures (including triplexes and quadruplexes) can be particularly useful for separating and detecting macromolecules, complexes and biological targets (e.g., soluble proteins, peptides, viruses, microbes and cell surface markers). Libraries containing sequence variants were used to favor selectivity for a particular target molecule or class of molecules with dissociation of ligand-target complexes performed under experimentally determined elution conditions guided by a general understanding of the salt dependency of oligonucleotide secondary structure. Representative examples of target molecules and associated applications include isolation of fatty acid binding proteins, purification of progenitor cells expressing different surface markers, protein sorting as a preparative step for proteomic analysis using 2D electrophoresis followed by mass spectrometry and identification of heparin mimetics for affinity chromatography to separate coagulation factors, nucleic acid binding proteins, lipoprotein lipases, protein synthesis factors, growth factors and actin-binding proteins. Examples of switching mechanisms used to capture and release different types of target molecules include, e.g., capture sequences that switch between unimolecular quadruplexes and unimolecular duplexes that form binding sites for transcription factors (binding in LiCl with elution with KCl); capture sequences that switch between unimolecular quadruplexes and unimolecular triplexes that form binding sites for high molecular weight glycoproteins (binding in LiCl at low pH and elution with KCl at high pH; capture sequences that form unimolecular quadruplexes in the absence of target and that complex with target nucleic acid (e.g., miRNA) to form bimolecular duplexes (binding in LiCl and elution with KCl; and three-way junctions that transition between quadruplex and/or triplex conformations. Each of these examples illustrates the complementary principles of 1) enhancing selectivity by balancing conformational states with different affinities and 2) designed engineering of elution switches.

### Example 16

### Detection of Cy3-Labeled Anti-IgG TAL Binding to Mouse IgG by Fluorescence Polarization

A library of duplex, triplex and quadruplex-containing oligonucleotides was prepared and screened for IgG binding activity using fluorescein-labeled mouse IgG. Seven TAL candidates were selected for solution-phase analysis by fluorescence polarization (see, for sequences of TALs RAD24-RAD30). Cy3-labeled TALs (10 nM) were incubated with polyclonal mouse IgG (1 μM) or IgG-free serum for 60 minutes at room temperature in 200 μL reaction mixtures buffered with either 20 mM phosphate-buffered saline, pH 7.0 or 20 mM acetate buffer, pH 5.8, containing 1 mg/ml MgCl. Fluorescence was measured at 15 minute intervals using a FarCyte Plate Reader (Amersham Pharmacia, Piscataway, N.J.). The percent change in fluorescence polarization was calculated from the mean of determinations in the presence and absence of mouse IgG. Data obtained in phosphate buffered saline, pH 7.0, are presented in Table 1.

TABLE 1
Fluorescence polarization of Cy3-
labeled anti-IgG TALs by mouse IgG.

| | Sequence | Polarization |
|---|---|---|
| TAL | (Cy3-labeled at 5′-end) | at 45′ (%) |
| RAD24 | /5Cy3/TCC TCT TCT TTT TCT TCT C | 21 |
| RAD25 | /5Cy3/TTC CTT CCT TCC TTC CTT C | 22 |
| RAD26 | /5Cy3/TCT CTC TCT CTC TCT CTC T | 38 |
| RAD27 | /5Cy3/TCC TTT CCT TTC CTT TCC T | 22 |
| RAD28 | /5Cy3/TCC CTT TCC CTT TCC CTT | 22 |
| | TCC C | |
| RAD29 | /Cy3/AGG CCG CGC CCC CCG CGC | 19 |
| | CCA CCG CCC CGG TGC C | |
| RAD30 | /5Cy3/GGA GGT GCT CCG AAA GGA | 15 |
| | ACT CC | |

The percent change in polarization of the RAD26 TAL was significantly greater than others. Similar results were obtained in 20 mM sodium acetate, pH 5.8, except that changes in polarization ranged from 9.5% to 40%. RAD26 again showed the greatest IgG-dependent change in polarization, consistent with experiments in phosphate buffer.

### Example 17

### IgG Detection by Fluorescence Microplate Assay Using Anti-IgG TAL Captured by IgG Immobilized on Paramagnetic Particles

Mouse IgG was immobilized on one micron paramagnetic particles at room temperature according to the following protocol. Amine-modified BIOMAG (Advanced Magnetics) was washed five times with vigorous vortexing and magnetic separation in 10 mM sodium phosphate (10 mM sodium phosphate, pH 7.35) at a particle concentration of 10 mg/ml. After the final wash, the wet cake was resuspended to 25 mg/ml in 6.25% glutaraldehyde (Sigma-Aldrich, St. Louis, Mo.) and rotated at room temperature for 3 hours.

Glutaraldehyde-treated particles are washed five times in sodium phosphate and once in 20 mM sodium acetate, pH 5.8 plus 1 mM MgCl2 (binding buffer) containing mouse IgG at 10 mg/ml to yield 160 μg IgG per mg BIOMAG. An aliquot of the IgG-containing solution is retained for determination of immobilization efficiency. The protein-particle slurry is rotated at room temperature for 16 hours. Particles are magnetically separated, and the supernatant is decanted and retained for estimation of residual IgG. Particles are resuspended to 10 mg/ml in 1 M glycine (pH 8.0) followed by rotation for one hour to quench unreacted glutaraldehyde groups. Quenched particles are washed twice in binding buffer and blocked by rotation for two to four hours in binding buffer containing 1 mg/ml bovine serum albumin to block exposed regions of the particle surface. Blocked particles are washed three times in binding containing 1 mg/ml bovine serum albumin, resuspended to a particle concentration of 10 mg/ml and stored at 2-8° C. Working aliquots are washed three times in binding buffer with thorough vortexing at a particle concentration of 1 mg/ml prior to use to protect against leaching of immobilized IgG with prolonged storage.

Sandwich assays are performed in black, flat-bottomed polystyrene microtiter plates (Dynatech Laboratories, Arlington, Va.) with bottom pull magnetic separation. Varying concentrations of purified mouse IgG (200 μl containing 1 ng/ml to 10 μg/ml IgG vs. IgG-free buffer) are preincubated for 30 minutes with 200 μl of 5′-biotinylated anti-mouse IgG TAL (10 nM). 5′-biotinylated nonsense oligonucleotide is incubated with IgG-containing and IgG-free buffer as a negative control. Duplicate 50 μl aliquots of each reaction mixture are pipetted into wells followed by addition of 50 μl of immobilized mouse IgG particles (50 μg/well). Plates are incubated for 60 minutes at room temperature with gentle shaking. Particles are washed twice in binding buffer and incubated for 60 minutes with gentle shaking in 50 μl binding buffer containing 1 μg/ml phycoerythrin-labeled streptavidin (Columbia Biosciences, Columbia, Md.). Particles are then washed twice and resuspended in 200 μl binding buffer, and fluorescence at 573 nm is measured with 488 nm excitation in a Fluorolite 1000 Microplate Fluorometer (Dynatech Laboratories, Arlington, Va.). Fluorescence readings indicate maximal binding in IgG-free wells with dose-dependent decreases in binding as a function of the concentration of mouse IgG. Particles are then washed twice with 200 μl of 50 mM Tris, pH 8.3 plus 100 mM KCl (release buffer) and resuspended in 200 μl of the same buffer. Fluorescence readings show no statistically significant difference from background (biotin-labeled nonsense oligonucleotide), indicating that streptavidin-biotin-TAL complexes are dissociated from wells by the release buffer washes.

### Example 18

### TAL Sensor-Based Detection of Thrombin Using a Photodiode Transducer

Thrombin (5 μg/ml in 10 μL carbonate/bicarbonate buffer, pH 9/6) is passively adsorbed to the hydrophobic surface (approximately 4 mm²) of polymer-coated indium phosphide photodiodes selected for maximal responsiveness (signal-to-noise ratio) at 560-600 nm. Photodiodes are then washed in SSC buffer and air dried. Ten μl Cy5-labeled inosine-variant anti-thrombin TAL is added at concentrations ranging from 1-100 nM in TEAA buffer containing 200 mM LiCl in the presence and absence of 1 μM thrombin. Specific, dose-dependent binding of the Cy5-labeled anti-thrombin TAL is detected as electrical current of thrombin-free Cy5-labeled TAL samples compared to thrombin-containing samples following photodiode excitation through a 550/25 nm band pass filter. In the absence of solution-phase thrombin, specific binding of Cy5-labeled TAL is measured as a voltage-dependent current response of the photodiode to Cy5 emission at 570 nm compared with background fluorescence in thrombin-containing samples. Photodiodes are then washed three times in TEAA buffer containing 10 mM KCl, and fluorescence measurements are repeated. No significant difference is detected in fluorescence of thrombin-free samples compared to thrombin-containing samples following KCl washes. Subsequent titration of thrombin (1 nM to 1 μM) in samples containing 200 mM LiCl and 10 nM Cy5-labeled TAL shows dose-dependent decreases in current as a function of thrombin concentration, indicating that the anti-thrombin TAL has been switched from a thrombin-nonbinding to a thrombin-binding state by the change in solution conditions from 10 mM KCl to 200 mM LiCl.

### Example 19

### TAL Sensor-Based Detection of Thrombin Using an Optical Waveguide Transducer

Affinity purified mouse IgG (OEM Concepts, Toms River, N.J.) is immobilized on 1×60 mm cylindrical quartz fibers with polished ends by passive adsorption in a 10 mM carbonate-bicarbonate (pH 9.6) buffer for two hours at room temperature. Coated fibers are blocked for one hour in 20 mM sodium acetate, pH 5.8 plus 1 mM MgCl2 (binding buffer) containing bovine serum albumin (1 mg/ml), washed thoroughly with binding buffer containing and air-dried prior to use in binding assays. Specific binding of anti-mouse IgG TAL (RAD26) 5′-labeled with Cy5 to IgG-coated fibers in the absence and presence of 10 ug/ml mouse IgG is detected through evanescent excitation of bound anti-mouse IgG TAL and evanescent capture of emitted energy using a portable fluorometer (ORD Inc., North Salem, N.H.) equipped with 550 nm excitation and 570 nm emission band-pass filters. Fibers are mounted vertically in a flow cell having a capacity of 50 μl and perfused with binding buffer at a rate of 200 μl/minute. Fluorescent light is collected and guided by the fiber and detected by photodiodes arranged so as to distinguish between surface-bound fluorescence (from smaller angles) and background light (from larger angles). The transducer in this example is the optical fiber operatively coupled through its evanescent field to photodiode(s) capable of generating an electronic signal (voltage). The fiber is then washed in 50 mM Tris, pH 8.3 plus 100 mM KCl (release buffer) and optical measurements are repeated. Measurements in mouse IgG-free release buffer compared with mouse IgG-containing release buffer show background level voltage, indicating that binding of the labeled anti-mouse IgG TAL does not occur in the KCl-induced state of the TAL.s. Fibers are then washed thoroughly in binding buffer, and the experiment is repeated. Mouse IgG-specific binding is again detected, demonstrating that the buffer-dependent change in TAL conformational state is reversible. This example illustrates use of an optical waveguide-based sensor to detect IgG-specific binding of the anti-mouse IgG TAL.

### TAL-Based Separation and Purification of Native, Modified and Conjugated Antibodies and Antibody Fragments

Disclosed in this section are multistate affinity ligand-based reagents, methods, devices, systems and media for the separation and purification of antibodies, antibody fragments and conjugates of antibodies and antibody fragments. These embodiments of the invention relate to the field of antibody purification. Purification of antibodies from complex mixtures is particularly challenging, as it may be preferable to retrieve all immunoglobulins from a particular sample or, alternatively, to selectively isolate or discriminate immunoglobulins of a particular class, subtype or binding property. Furthermore, established chromatographic methods for antibody purification using immobilized Protein A and Protein G require elution under acidic conditions that have been shown to cause aggregation, precipitation, denaturation and destabilization of antibody molecules. Compositions and methods of making and using multistate affinity ligands are described here for the gentlest possible purification of antibodies and antibody conjugates without exposure to acidic conditions. Purification using multistate affinity ligands is achieved in a manner that allows for separation of all immunoglobulins from a sample or only immunoglobulins of a particular type or species, optionally using ligands that bind to a particular region of the immunoglobulin molecule. These multistate affinity ligands are rationally designed to switch between conformational states that bind and release antibodies and antibody conjugates under conditions that do not perturb antibody or conjugate structure or function. Commercial applications include production and processing of high-value antibodies and antibody conjugates for research, industrial, diagnostic and therapeutic applications.

In one embodiment of the present invention, a medium for purifying a target molecule selected from the group consisting of antibodies, antibody fragments and conjugates thereof comprises a nucleotide-containing multistate affinity ligand immobilized on a matrix. The multistate affinity ligand exists in a first state having a defined first affinity for the target molecule in a first buffer and a second state having a defined second affinity for the target molecule in a second buffer wherein the ratio of the defined first affinity to the defined second affinity is at least two.

In another embodiment of the present invention, a preparative device for isolating target molecules from a sample (the target molecules being selected from the group consisting of antibodies, antibody fragments and conjugates thereof) comprises:

a) a nucleotide-containing multistate affinity ligand;

b) means for delivering the sample to the multistate affinity ligand to form a reaction mixture in which the multistate affinity ligand exists in a target-binding state;

c) means for partitioning ligand-target complexes from other substances in the reaction mixture;

d) means for converting the multistate affinity ligand from a target-binding state to a target-nonbinding state; and

e) means for partitioning unbound target molecules from ligand-bound target molecules.

In another embodiment of the present invention, a kit for the purification of an antibody, antibody fragment or conjugate thereof comprises a buffer-responsive multistate affinity ligand, a binding buffer and a releasing buffer. The multistate affinity ligand comprises a nucleotide-containing polymer that switches between an immunoglobulin-binding state in the presence of the binding buffer and an immunoglobulin-nonbinding state in the presence of the releasing buffer.

In another embodiment of the present invention, a system for purifying from a sample a target molecule selected from the group consisting of antibodies, antibody fragments and conjugates thereof comprises:

a) a processing reservoir containing a separation reagent;

b) input means for delivering substances to the processing reservoir;

c) output means for removing substances from the processing reservoir;

d) a first buffer solution; and

e) a second buffer solution;

wherein the separation reagent is a nucleotide-containing multistate affinity ligand that exists in a first state with a relatively high affinity for the target molecule in the presence of the first buffer solution and a second state with a relatively low affinity for the target molecule in the presence of the second buffer solution.

In another embodiment of the present invention, a method of purifying an antigen-binding target molecule from a sample containing the target molecule comprises:

a) contacting the sample with an environmentally-sensitive multistate affinity ligand under a first environmental condition;

b) partitioning the ligand-target complex from nontarget substances in the sample; and

c) releasing the target from the ligand-target complex by exposing the complex to a second environmental condition

wherein
i) the target molecule is selected from the group consisting of antibodies, antibody fragments and conjugates thereof;
ii) the antigen-binding properties of the target molecule remain intact following exposure to the first environmental condition and the second environmental condition; and
iii) the multistate affinity ligand comprises a nucleotide-containing polymer that reversibly partitions between a first state having a first affinity for the target molecule under the first environmental condition and a second state having a second affinity for the target molecule under the second environmental condition.

In another embodiment of the present invention, a method of separating a first molecule comprising an antibody, antibody fragment or conjugate thereof from a second molecule comprises:

a) contacting a sample containing the first molecule and the second molecule with a nucleotide-containing immobilized multistate affinity ligand in a first buffer solution having a composition in which the multistate affinity ligand exists in a first state that specifically binds the first molecule with relatively high affinity;

b) incubating the sample with the immobilized multistate affinity ligand for a sufficient contact time to allow the immobilized multistate affinity ligand to bind the first molecule to form an immobilized ligand-first molecule complex;

c) partitioning the second molecule from the immobilized ligand-first molecule complex;

d) exposing the immobilized ligand-first molecule complex to a second buffer solution having a composition in which the immobilized multistate affinity ligand has a relatively low affinity for the first molecule; and

e) partitioning the first molecule from the immobilized multistate affinity ligand.

In another embodiment of the present invention, a method of making an antibody purification product comprises immobilizing a multistate affinity ligand on an insoluble matrix and packaging the immobilized multistate affinity ligand in a sealed or sealable container. The multistate affinity ligand comprises a nucleotide-containing polymer that specifically binds in a first buffer to an antigen-binding target molecule selected from the group consisting of antibodies, antibody fragments and conjugates thereof to form an immobilized multistate affinity ligand-target complex that dissociates in a second buffer to yield ligand-free target molecule.

In another embodiment of the present invention, a method of separating a first molecule or group of molecules selected from the group consisting of antibodies, antibody fragments and conjugates thereof from a second molecule comprises the steps of:

a) contacting a sample containing the first molecule or group of molecules and the second molecule with a nucleotide-containing multistate affinity ligand immobilized on a solid support immersed in a binding buffer;

b) incubating the sample with the immobilized multistate affinity ligand for a sufficient contact time to allow the immobilized multistate affinity ligand to bind the first molecule or group of molecules to form an immobilized ligand-molecule complex;

c) performing a rinsing step to remove the second molecule;

d) performing at least one elution step to dissociate the first molecule or group of molecules from the ligand of the immobilized ligand-molecule complex; and

e) collecting at least one product of the at least one elution step;

wherein said at least one elution step causes the multistate affinity ligand to shift from a first conformational equilibrium state that favors association of immobilized ligand-molecule complexes to a second conformational equilibrium state that favors dissociation of immobilized ligand-molecule complexes.

In another embodiment of the present invention, a medium for purifying target molecules selected from the group consisting of antibodies, antibody fragments and conjugates thereof comprises a support-bound plurality of ligands, said plurality of ligands including at least one multistate affinity ligand existing in a first state having a defined first affinity for a target molecule in a first buffer and a second state having a defined second affinity for the target molecule in a second buffer wherein the ratio of the defined first affinity to the defined second affinity is at least two.

In another embodiment of the present invention, a method of making an antibody purification product comprises preparing a support-bound plurality of ligands including at least one multistate affinity ligand and packaging the support-bound plurality of ligands in a sealed or sealable container. Said plurality of ligands including at least one multistate affinity ligand comprises a nucleotide-containing polymer that specifically binds in a first buffer to antigen-binding target molecules selected from the group consisting of antibodies, antibody fragments and conjugates thereof to form support-bound multistate affinity ligand-target complexes that dissociate in a second buffer to yield ligand-free target molecules.

The description and examples that follow relate to the separation of antibodies, antibody fragments and conjugates thereof using multistate affinity ligands rationally designed and selected to undergo analytically and functionally definable conformational transitions from a first affinity state under a first operator-defined environmental condition to a second affinity state under a second operator-defined environmental condition. The multistate affinity ligands of the invention are tunable in the sense that the structural transition of a multistate affinity ligand from a first conformational state to a second (or third or fourth, etc.) conformational state can modulated in a controlled manner by well-defined changes in environmental conditions. Each conformational state of the multistate affinity ligand has a measurable affinity for a particular target antibody, antibody fragment or conjugate thereof under a particular environmental condition. The difference in affinity of the different conformational states of the multistate affinity ligand for it's the particular target antibody, antibody fragment or conjugate thereof can be used to achieve highly selective separations of populations and subpopulations of target molecules from one another and from nontarget species in specimens, samples and complex mixtures such as biological isolates, culture media, conjugation reactions and the like.

In the present invention, a multistate affinity ligand capable of existing in a first state having a first affinity for a specified antibody and also capable of existing in an alternative second state having a second affinity for said antibody is utilized for purification of specific antibodies, antibody fragments, and conjugates of antibodies and conjugates of antibody fragments. Said multistate affinity ligand may be included in compositions, articles, and methods, including methods, kits, devices, and systems.

A new method is disclosed herein for separating a target (such as antibodies, antibody fragments, antibody conjugates and/or antibody fragment conjugates, e.g., IgG and/or other related immunoglobulins and immunoglobulin-derived proteins) by using multistate affinity ligands. Multistate affinity ligands are polymeric ligands, synthesized completely or in part by solid phase synthesis methods, and incorporating environmentally sensitive conformational switches. An essential feature of multistate affinity ligands is that under defined conditions the target-binding affinity for binding to a given multistate affinity ligand conformation differs by a measurable degree from binding to another multistate affinity ligand conformation. Multistate affinity ligands are designed to incorporate monomer sequences that have propensities to switch among two or more different conformations, Conformation may be defined by physical measurements that include spectroscopic, hydrodynamic and thermodynamic techniques and by modeling of solution-dependent binding characteristics.

For chromatographic or other separation applications, interactions to surface-attached multistate affinity ligands are modulated by shifting multistate affinity ligand conformational equilibria by using mild changes in solution conditions. The resultant modulation in binding affinity to different targets enhances the ability to obtain high resolution separations.

The method comprises 1) attaching a multistate affinity ligand to a solid support, 2) allowing the surface-attached multistate affinity ligands to interact under binding conditions to a mixture containing one or more distinguishable targets such as antibodies, antibody fragments, antibody conjugates and/or antibody fragment conjugates, e.g., an IgG species, 3) rinsing the solid support under binding conditions to remove unbound or weakly bound contaminants, and 4) eluting from the support using a continuous gradient, or a combination of continuous and step gradients wherein the elution buffer switches the multistate affinity ligand from a conformation or conformations that favor binding to a conformation or conformations that disfavors binding.

Components of the device and method for separating specific target molecules such as antibodies, antibody fragments, antibody conjugates and/or antibody fragment conjugates, e.g., IgG molecules and/or other related immunoglobulins and immunoglobulin-derived proteins, from contaminating material and from other antibody, antibody fragment, antibody conjugate and/or antibody fragment conjugate molecules are briefly described below.

First, a nucleotide-containing oligomeric or polymeric molecule (multistate affinity ligand) is needed that exists in an equilibrium between two or more states. The distribution of the multistate affinity ligand conformations among the accessible equilibrium states is controlled by solution conditions including, but not limited to, the concentrations and nature of salts and other small-molecule effectors, the pH and the temperature. The conformational state of the multistate affinity ligand is defined by physical measurements that are familiar to those skilled in molecular biophysics, polymer chemistry, biochemistry and molecular biology and include, but are not limited to, NMR spectroscopy, UV spectroscopy, CD spectroscopy, calorimetry, hydrodynamic, chromatography and electrophoresis.

Second, a solid support is needed, together with a means for attaching the multistate affinity ligand to the support. For example, the solid support may be chromatographic beads or other media functionalized for attachment, e.g., to primary amines, sulfhydryl groups or biotin labels. The ligand is, in turn, synthesized to have terminal or internal reactive groups to allow functional attachment to the solid support.

Third, buffers and elution conditions are needed in order to 1) facilitate binding and 2) to switch ligand conformation and facilitate release. The minimum requirements are a binding buffer and a release buffer that can be defined in various ratios in continuous or step gradients in order to bind and release target molecules (such as antibodies, antibody fragments, antibody conjugates and/or antibody fragment conjugates, e.g., IgG and/or other related immunoglobulins and immunoglobulin-derived proteins) under controlled conditions.

Finally, additional buffers may be needed to wash the solid support following elution and to regenerate and store the solid support for future separations.

Steps in separating target molecules (such as antibodies, antibody fragments, antibody conjugates and/or antibody fragment conjugates, e.g. IgG molecules and/or other related immunoglobulins and immunoglobulin-derived proteins) from contaminating material and from other antibodies, antibody fragments, antibody conjugates and antibody fragment conjugates, such as, e.g., other IgG molecules. The method for separating target antibodies, antibody fragments, antibody conjugates and/or antibody fragment conjugates (such as, e.g., specific IgG proteins) from other antibodies, antibody fragments, antibody conjugates and/or antibody fragment conjugates (such as, e.g., other IgG proteins and related immunoglobulin-derived proteins) from each other and from undesirable contaminants comprises 1) attaching a nucleotide-containing multistate affinity ligand to a solid support, 2) allowing the surface-attached multistate affinity ligand to interact under binding conditions with a mixture containing one or more distinguishable antibodies, antibody fragments, antibody conjugates and/or antibody fragment conjugates, such as a specific IgG species, 3) rinsing the solid support under binding conditions to remove unbound or weakly bound contaminants, and 4) eluting from the support using a continuous gradient, step gradients or a combination of continuous and step gradients wherein the elution buffer switches the multistate affinity ligand from a conformation or conformations that favors binding to a conformation or conformations that disfavors binding, and 5) re-equilibration of the column with binding buffer. Additional steps useful for reusable separations material comprise 5) rinsing with a wash buffer(s) to clean and de-contaminate the column and 6) rinsing and storing with a storage buffer to maintain the support in functional form. The rinse buffer may be, e.g., a mildly basic solution of sodium hydroxide or a detergent solution to sterilize and remove aggregated proteins. The storage buffer may contain, e.g., low concentrations of toxic or antibiotic material to maintain sterile conditions.

Performance characteristics and advantages of the method over currently used methods. The multistate affinity ligand separations method is superior to existing methods involving Protein A and Protein G in several respects. First, the method is able to purify all known subtypes of, e.g., IgG from species including, e.g., human, mouse, goat and rabbit. For example, the method is able to purify human subtype 3, which is weakly bound to Protein A and cannot be purified using Protein A columns. Second, in contrast to Protein A and G purifications, which involve partial denaturation of target molecules, such as, e.g., IgG, the multistate affinity ligand method is intrinsically mild and nondenaturing. Because of the partial denaturing conditions required for purifications involving Protein A and G, some IgG purifications involving these ligands result in unacceptably large losses of sample IgG and the purified IgG's antigen binding activity. Third, in contrast to a protein ligand such as Protein A or G, multistate affinity ligands are robust ligands which can be subjected to rather harsh washing conditions, including washing with both dilute NaOH and with detergents. Fourth, in contrast to protein ligands such as Protein A or G, which provide only limited ability to fractionate IgG subtypes from each other (and then only from partially purified samples) methods involving multistate affinity ligands can separate different IgG species from each other even from crude IgG-containing mixture. For example, multistate affinity ligand methods allow the separation of various human IgG subtypes from each other as well as resolution of immunoglobulins from different host species, e.g., separation of fetal calf IgG from human IgG. Multistate affinity ligands also allow the separation of IgG based on the number and type of conjugated molecules within an antibody conjugate, e.g., the number and type of fluorescent dyes with which an antibody is labeled.

Media preparation. Ligand is attached to, e.g., 90 micron particles sold in bulk, 30 micron beads sold in pre-packed columns of various sizes for general laboratory use or 5-10 micron particles comprising high performance media for use with HPLC and proteomics applications. In addition, other possible small preparation formats include, e.g., ligand bound to membrane filters for quick and easy clean-up of culture broths and for concentration of the monoclonal IgG.

Buffers. In addition to regular process buffers for IgG binding and recovery, additional buffers include, e.g., those specifically selected for the removal of contaminating immunoglobulins (e.g., bovine IgG) from target immunoglobulins (e.g., monoclonal IgG produced in cell culture).

Advantages. The multistate affinity ligand-based process results in recovery of activity and the reduction of aggregates caused by elution with denaturing conditions, thereby producing a highly uniform and reproducible IgG product.

The use of TALs for the separation and purification of antibodies, antibody fragments and conjugates of antibodies and antibody fragments is illustrated in the following examples, which describe certain embodiments of the invention and are not intended to be limiting.

### Example 20

### Screening of Hairpin and Quadruplex Forming Oligonucleotides by Filtration of Sepharose-Bound IgG

78 different hairpin- and quadruplex-forming oligonucleotides were synthesized and aliquoted into a 96-well microplate. Samples of each of these oligonucleotides were screened for IgG binding in 96-well silent screen plates with 3.0 um pore size Loprodyne membrane. For each of the oligonucleotides, two sets of individual aliquots (100 uL in volume) of equimolar concentration were prepared for screening. A 10 uL suspension of mixed human IgG bound to Sepharose beads was added to one of the individual aliquots, incubated for 20 minutes and filtered through the screen. Individual aliquots were filtered through the membrane under vacuum and collected on 96-well UV-plates. The IgG-derivatized Sepharose beads were retained on the plate along with the bound DNA, and the unbound oligonucleotide passed through the screen. Both plates were read in a plate reader for the difference in optical density (OD) reading, which served as an indicator of binding. The experiment was repeated with various buffer and salt conditions. Two significant hits, at well positions C7 and H2, were identified based on interaction with the IgG-Sepharose beads. Eight other hits of lesser binding activity were also identified. Of the lead sequences, d(TTTTCGCGCGTTTCCGCGCGAA) was designed to form a hairpin, and d(TTTTGGTTGGGGTGGTTGG) was designed to form a quadruplex. Among the other eight oligomers, six of them were hairpins, and the rest were potential quadruplexes.

### Example 21

### Reverse-Screen Experiment Identifies a Lead Compound

C7, H2 and the control oligomer d(TGTGTGTGTGTGTGT) were synthesized with terminal 5′ aminohexyl groups and were used to derivatize activated Sepharose beads. The retention of IgG and the IgG fragment Fab′2 proteins on immobilized C7, H2, (TG)7T and ethanolamine Sepharose beads was determined on 96-well filter plates (3.0 micron pore size) in a buffer containing 100 mM TEAA, 20 mM Mg²⁺, pH 7. The objectives were a) to distinguish between normal protein retention on the screen, Sepharose, immobilized regular oligonucleotides, and the immobilized multistate affinity ligands, and b) to validate the previous plate assays, between immobilized IgG, and free multistate affinity ligands. For each concentration of protein, two sets of individual aliquots (150 uL in volume) were prepared for screening. Six different stock solutions of each protein were prepared for this assay. For the standard curve, each concentration of the protein was used in triplicate, and directly added to the 96-well UV plate. A 10 uL suspension of DNA bound to Sepharose beads was added to one of the individual aliquots, incubated for 20 minutes and filtered through the screen. Each set of the individual aliquots was filtered through the membrane under vacuum and collected on 96-well UV-plates. The protein bound on the beads was retained on the plate, and unbound protein passed through the screen. In order to determine protein concentrations, freshly prepared BCA reagent was added to each well (150 uL), incubated for 2 hrs at 35 C, and the absorbance was measured at 562 nm. Standard curves of different concentrations of protein in BCA reagent were determined for comparison.

Upon analysis of the filtrate (protein concentration) in each well, the degree of retention was as follows H2>C7>>TG repeat>capped ethanolamine (Sepharose used as blank screen). Hence both the above multistate affinity ligands had greater affinity for the proteins than other oligonucleotides and blank beads, which validated our above results.

### Example 22

### Biophysical Signatures of Lead Compound Suggest the Possible Role of a Triple-Helical Structure

The CD spectra of H2 revealed the presence of a secondary structure for H2 in the presence of magnesium ion with a positive peak at 258, and a smaller positive peak at 295. Upon Fab′2 binding, the peak at 295 grew bigger with time. Titration of H2 into IgG and Fab′2 had a larger effect on the intrinsic fluorescence of the proteins in the presence of Mg²⁺ than in the absence. Since under the conditions of these experiments, Mg²⁺ is expected to destabilize quadruplexes, the Mg²⁺ effect suggested a potential alternative structure, e.g., a triplex structure. Triplexes are well-known to be stabilized by the presence of Mg²⁺.

Experimental Details For the CD experiments, the standard solution conditions were 20 mM PIPES, 2 mM Mg²⁺, 20 mM K⁺, pH 6.1. The data were acquired using an Aviv model 62DS spectropolarimeter (AVIV Instruments, Lakewood, N.J.) using 1.0 mm strain-free Quartz cuvettes. Samples were thermostatically controlled at 25 C and contained at least 20 uM multistate affinity ligand. Samples were scanned from 340 nm to 200 nm at 0.2 nm intervals, using a 20 sec averaging time.

### Example 23

### A Triplex 31mer Shows Favorable Binding Properties

The triplex 31mer 5′-CCTCTTC-TTTTT-CTTCTCC-TTTTT-GGAGAAG-3′ was synthesized and tested for binding to IgG and to IgG fragments. As observed using fluorescence spectroscopy, when the 31 mer was titrated in IgG, the intrinsic fluorescence quenched upon multistate affinity ligand binding. In fact, the 31 mer quenched the intensity more and increased the melting temperature by 3 C over H2 at pH 6.0. The UV melting data revealed that at lower pH in the presence of Mg²⁺, the triplex was predominant. Circular dichroism (CD) measurements verified triplex formation and the interaction with IgG. The signature trough around 216 nm indicated the formation of triplex.

### Example 24

### Behavior of Different Multistate Affinity Ligands with Respect to IgG Binding as Measured by Ultrafiltration

Eleven oligonucleotides were designed and synthesized to represent molecules that can potentially undergo conformational transitions involving quadruplexes, triplexes and three-way junction structures. Members of this primary set of oligonucleotides are listed and described in Table 2.

TABLE 2
Eleven molecules chosen for initial screening experiments.

| Name | Sequence | potential conformations | Major effectors |
|---|---|---|---|
| RAD1 | CCT CTT C(HEG)CT TCT CC(HEG)G GAG AAG | YYR triplex HEG linkers | Mg²⁺, pH, NaCI |
| RAD2 | CCT CTT CTT TTT CTT CTC CTT TTT GGA GAA G | YYR triplex | Mg²⁺, pH, NaCl |
| RAD9 | CTC TCT CTT TTT CTC TCT CTT TTT GAG AGA G | YYR triplex | Mg²⁺, pH, NaCl |
| RAD7 | GAG AGA GTT TTT GAG AGA GTT TTT CTC TCT C | RRY triplex | Mg²⁺, NaCI |
| RAD3 | TGG TTG GTT TTT GGA AGG ATT TTT TCC TTC C | RRY triplex/quadruplex | Mg²⁺, KCI, LiCI |
| RAD6 | GGA AAG GTT TTT GGA AAG GTT ITT CCT TTC C | RRY triplex/quadruplex | Mg²⁺, KCI, LiCI |
| RAD10 | TGG GCC GGT AAC GGG TTA CCG TAA GGT CCC | 3 way junction/quadruplex | Mg²⁺, KCI, LiCI |
| RAD11 | TGG GCC GGT AAC GGA TTA CCG TAA GGT CCC | 3 way junction/quadruplex | Mg²⁺, KCI, LiCI |
| RAD4 | CCC TCC CTG GGC TTT TTT TGA TTT TTC TTA A | CONTROL | |
| RAD5 | GAG TGA GTC TCA GTT AGT TTC GAT TGA TTC T | CONTROL | |
| RAD8 | TGG AGT CTG CGC GAG TCA GCG CTC AAG ATC | CONTROL | |

The molecules shown in Table 2 were screened for mixed human IgG binding on 96-well ultrafiltration plates from Millipore (MSNUO3010), using a vacuum device to draw samples through the membrane. IgG samples (ChromPure Human IgG) were obtained from Jackson ImmunoResearch Laboratories (West Grove, Pa.). These ultrafiltration plates allow multistate affinity ligands to pass through with a retention of less than 20%, but prevent IgG from passing through with retention of greater than 10%. These retentions were determined experimentally, under the buffer conditions of our measurements. The experimental protocol is as follows. A 200 microliter solution containing buffer, IgG and multistate affinity ligand were mixed, and filtered. IgG concentrations ranged from 0.1 μM to 2 μM, and multistate affinity ligand concentrations ranged from 20 nM to 100 nM. Standard solutions of multistate affinity ligand alone were also filtered, covering the experimental range of 20 nM to 100 nM. 50 microliter aliquots of the eluate from each filtration were added to three separate 150 μl test solutions containing 100 nM YOYO-1 dye, 150 mM NaCl, 15 mM sodium citrate, 10 mM CHAPS, pH=7.0, and 100 nM of either YOYO-1 dye or BOBO-3 dye (Invitrogen, Carlsbad, Calif.). The fluorescence intensities of each test solution were measured in a 96-well plate format, using a FarCyte plate reader (Amersham Pharmacia, Piscataway, N.J.) with filters at 485 nm for excitation and 535 nm for emission for the YOYO-1 measurements and with filters at 544 nm and 595 nm for the BOBO-3 measurements. The intensity readings from filtrates of the standard multistate affinity ligand concentrations were plotted vs. multistate affinity ligand concentration, and data points were fitted with a straight line. The multistate affinity ligand intensity from filtrates in the presence of IgG were compared to these standard curves and used to determine the amount of free IgG in these filtrates. By subtracting this number from the total concentration of IgG in the initial solution, the amount of oligonucleotide bound to IgG was obtained. From these measurements, the association equilibrium constant for oligonucleotide binding was obtained using the equation Kₐ=[PD]/[D]*[P], where [PD] is the concentration of bound oligonucleotide, [D] is the concentration of free oligonucleotide and [P] is the concentration of free IgG. Some results of the ultrafiltration determinations of multistate affinity ligand binding to mixed human IgG (ChromPure, Jackson ImmunoResearch Laboratories, West Grove, Pa.) are shown in Table 3 under the defined solution conditions given in the table. The results shown in Table 3 were obtained using BOBO-3 dye. Substantially similar results were obtained with YOYO-1 dye.

TABLE 3
Screening results of eleven conformationally diverse multistate
affinity ligands for binding to IgG, sorted by binding at pH 6.¹

| | | % bound | % bound | logKa | logKa |
|---|---|---|---|---|---|
| Description | name | pH 6 | pH 7 | pH 6 | pH 7 |
| YYR triplex | RAD 9 | 60 | 35 | 7.02 | 6.51 |
| YYR triplex | RAD 1 | 59 | 55 | 7.01 | 6.94 |
| HEG linkers | | | | | |
| YYR triplex | RAD 2 | 57 | 48 | 6.96 | 6.78 |
| CONTROL | RAD 4 | 53 | 38 | 6.89 | 6.58 |
| RRY triplex | RAD 7 | 52 | 33 | 6.86 | 6.47 |
| RRY triplex/ | RAD 6 | 51 | 35 | 6.84 | 6.51 |
| quadruplex | | | | | |
| CONTROL | RAD 5 | 50 | 34 | 6.82 | 6.49 |
| RRY triplex/ | RAD 3 | 44 | 38 | 6.71 | 6.58 |
| quadruplex | | | | | |
| 3 way junction/ | RAD 11 | 35 | 33 | 6.51 | 6.47 |
| quadruplex | | | | | |
| 3 way junction/ | RAD 10 | 26 | 27 | 6.31 | 6.32 |
| quadruplex | | | | | |
| CONTROL | RAD 8 | 20 | 16 | 6.13 | 6.01 |

¹Solution conditions: 0.15 M NaCl, 0.015 M sodium citrate, 1 mM MgCl₂. The multistate affinity ligand concentration was 100 nM and the IgG concentration was 200 nM.

### Example 25

### Triple-Helical Multistate Affinity Ligands as Tunable Ligands for Chromatographic Separation of Immunoglobulin G Antibodies: Effect of Loop Composition on Retention Times

The triple-helix forming multistate affinity ligand, RAD2 (see Tables 2 and 3) was synthesized with an aminohexane linker (C6 μm) on the 5′ end to give 5′-C6 μm-CCTCTTCTTTTTCTTCTCCTTTTTGGAGAAG-3′. This oligonucleotide was attached to Sepharose beads in a chromatography column using standard coupling chemistries. Briefly, the C6-amino terminal of the oligonucleotide was coupled with the n-hydroxy succinamide moiety of the column. The free NHS-activated groups were capped using ethanolamine. For comparison, variants of this multistate affinity ligand containing loop regions with hexaethylene glycol linkers and hexane linkers were also attached to Sepharose beads in a similar manner. These three chromatographic columns were compared for retention efficacy under gradient elution conditions that were designed to favor the tightly binding conformation at the beginning of the experiment and to favor the weakly binding conformation at the end of the chromatographic elution. Under these gradient conditions, the multistate affinity ligand variant with the hexaethylene glycol linkers, CCTCTTC(HEG)CT TCTCC(HEG)GGAGAAG, shows enhanced retention compared to the other variants (see FIG. 1). In this experiment, at time 0 a sample of fluorescein labeled human IgG (Jackson ImmunoResearch Laboratories, West Grove, Pa.) was injected onto the column, and fluorescence was monitored as a function of time using excitation at 490 nm and emission at 528 nm.

### Example 26

### Triple-Helical Multistate Affinity Ligands as Tunable Ligands for Chromatographic Separation of Immunoglobulin G Antibodies: Separation of IgG from Complex Samples.

The column prepared from the multistate affinity ligand variant with the hexaethylene glycol linkers, CCTCTTC(HEG)CTTCTCC(HEG)GGAGAAG, was further examined for its ability to separate IgG from complex biological samples, and the results were compared to separations of IgG from these samples performed using a Protein A-Sepharose column. The results of such a comparison are shown in FIG. 2, where the separation results for a serum sample run over a Protein A-Sepharose column is compared to those on our lead multistate affinity ligand-Sepharose column. The peak at 10.1 minutes collected from the Protein A-Sepharose column and the peak at 10.42 minutes collected from the multistate affinity ligand column were each electrophoresed over a 4-12% polyacrylamide gel using 1×SDS buffer and compared with IgG standards and molecular weight markers. After silver staining, only two bands were seen from each sample, one at about 50 kD, and another about 25 kD, as expected after breaking of all the disulfide linkage. The two bands from the multistate affinity ligand-purified sample corresponded with the two bands from the Protein A-purified sample and with the two bands of the IgG standard. The conclusion is that the purity of the multistate affinity ligand purified serum sample is indistinguishable from the purity of the Protein A purified sample as judged by SDS gel electrophoresis.

Also, as shown in FIG. 3, when material collected from the multistate affinity ligand peak is re-injected onto a Protein A-Sepharose column, most of the peak is retained at the position characteristic of IgG. The small amount of protein that comes through in the void volume appears to correspond to IgG subtype 3. In contrast to the Protein A column, our multistate affinity ligand column retains all IgG subtypes with comparable efficacy (see FIG. 4). Indeed, as seen in FIG. 4, the slightly longer retention time that is observed for the IgG3 subtype compared to the other subtypes suggests a modestly higher affinity of the multistate affinity ligand column for the IgG3 subtype compared to the other subtypes. Application of a shallower gradient represents an attractive approach to separating some or all of the IgG subtypes from each other.

A further demonstration of the ability of our multistate affinity ligand column to bind specifically to IgG is shown in FIG. 5, which shows the results of separations of fluorescein labeled IgG from 1) a sample containing labeled IgG plus BSA and 2) from a serum sample that was doped with fluorescein-labeled IgG. Interestingly, a comparison of the UV and fluorescence signals of the serum sample (which contains unlabeled IgG from the blood) suggests a partial resolution of labeled and unlabeled IgG, again with the application of a step gradient. This observation suggests that multistate affinity ligand technology can separate closely related proteins that differ only in the extent of fluorescent labeling.

The ability of our lead compound to separate human from mouse IgG has been examined. As shown in FIG. 6, our lead multistate affinity ligand column does bind tightly to mouse IgG, as it does to human IgG. Based on this observation, a process of multistate affinity ligand and gradient optimization is being developed for the separation of human from mouse antibodies.

### Example 27

### Multistate Affinity Ligand-Antibody Interaction Screening Assay

The purpose of work described in this example was to devise a rapid method to screen multistate affinity ligand interactions with target antibodies and antibody conjugates in a way that predicts the performance of multistate affinity ligands as chromatographic ligands.

Basic methodology. Individual multistate affinity ligands were mixed with the target antibodies or antibody conjugates in various solution environments. After a short incubation, the mixture was separated by ultrafiltration through a UF well plate that retains the target and the target-bound multistate affinity ligand. The filtrate containing the unbound multistate affinity ligand was collected and the multistate affinity ligand quantified by the use of a fluorescent dye which, when it interacts with the multistate affinity ligand, shows a large increase in fluorescence quantum yield. The fluorescence intensity of the multistate affinity ligand filtrate was measured by a fluorescence plate reader and quantified using a standard curve of fluorescence intensity related to multistate affinity ligand concentration. Filtrate with low fluorescence intensity indicates multistate affinity ligand binding to the target and, thus, potential for use as a chromatographic affinity ligand for the target antibody or antibody conjugate.

Methodological notes. The selection of the proper ultrafiltration well plate for screening was critical for the assay effectiveness. The UF plate must effectively separate the larger target and target-bound multistate affinity ligand from the free multistate affinity ligand. Also the UF membrane must exhibit high passage and low binding of the free multistate affinity ligand for proper quantification. Finally the vacuum filtration device must exhibit little cross contamination between filtrate wells. The Millipore (Billerica, Mass.) MultiScreen HITS PCR 96-Well Plate system best met these requirements. The UF well plate membrane retains protein to >90% and allows >98% recovery of unbound multistate affinity ligand in the filtrate. The design of the Millipore MSVM HITS vacuum manifold reduces cross contamination for filtered wells.

Selection of the best fluorescent dye for quantification of the multistate affinity ligand was a difficult task. The dye must show a large (2 orders of magnitude) increase in fluorescence upon interaction to the multistate affinity ligand to reduce background allowing detection low quantities (nanomolar). The fluorescence intensity should be linear over several orders of magnitude. Also, it is desirable to have the fluorescence intensity somewhat uniform independent of the composition of the multistate affinity ligand. The Molecular Probes (Eugene, Oreg.) dye Picogreen was the best compromise having the desired features of a detection fluor for multistate affinity ligand quantification. It was sensitive and showed linearity in the desired concentration range. However, Picogreen required individual calibration curves be established for individual multistate affinity ligands. It also showed a tendency to bind to the assay plate which had to be reduced by the addition of the detergent CHAPS to the fluorescence assay wells.

Experimental procedure. The fluorescence intensity versus multistate affinity ligand concentration standard curves were prepared for each multistate affinity ligand for every assay. Curves were prepared by filtering 200 microliters of a 100 nM, 50 nM and 20 nM multistate affinity ligand solution through the UF well plate, collecting the filtrate and making measurements in triplicate by taking 50 microliters of filtrate and mixing with 100 microliters of 0.1 micromolar Picogreen, 10 mM CHAPS solution. Measurements were made in a FARCyte fluorescence microplate reader (Amersham Pharmacia, Piscataway, N.J.) using a 485/20 nm excitation filter and a 535/25 emission filter.

A typical multistate affinity ligand-antibody interaction assay involved making a 200 microliter mixture containing multistate affinity ligand at a concentration of 100 nM and target antibody at a concentration of 200 nM, incubating at RT for 30 minutes and filtering through the UF well plate under 25 inches of Hg vacuum pressure. The filtrate was collected and triplicate assays for multistate affinity ligand in the filtrate were made with the addition of Picogreen in CHAPS as described above. The amount of free multistate affinity ligand in the filtrate was quantified from the standard curves prepared from the same filtration.

### Example 28

### Behavior of Different Multistate Affinity Ligands with Respect to Immunoglobulin Binding as Measured by Ultrafiltration Using a Picogreen Dye-Based Assay

Nineteen oligonucleotides were designed and synthesized to represent molecules that can potentially undergo conformational transitions involving a variety of forms. These oligonucleotides are listed and described in Table 4. The molecules were screened for immunoglobulin binding on MSNUO3010 96-well ultrafiltration plates from Millipore (Billerica, Mass.) using a vacuum device to draw samples through the membrane. These ultrafiltration plates allow multistate affinity ligands to pass through with a retention of less than 20%, but prevent antibodies and antibody fragments from passing through with retention of greater than 10%. These retentions were determined experimentally under the buffer conditions of our measurements. Polyclonal human and mouse IgG samples were obtained from Jackson ImmunoResearch Laboratories (West Grove, Pa.). Monoclonal IgM, IgA and IgG subtypes were obtained from Calbiochem (a subsidiary of EMD, Biosciences, Gibbstown, N.J.). The Molecular Probes (Eugene, Oreg.) dye Picogreen was used as a detection fluor for oligonucleotide quantification. It was sensitive and showed linearity in the desired concentration range. However, Picogreen required individual calibration curves be established for individual oligonucleotides. It also showed a tendency to bind to the assay plate, which nonspecific binding had to be reduced by the addition of the detergent CHAPS to the fluorescence assay wells. The experimental protocol was as follows.

The fluorescence intensity versus multistate affinity ligand concentration standard curves were prepared for each multistate affinity ligand for every assay. Curves were prepared by filtering 200 microliters of a 100 nM, 50 nM, and 20 nM multistate affinity ligand solution through the UF 96-well plate, collecting the filtrate and making measurements in triplicate by taking 50 microliters of filtrate and mixing with 100 microliters of 0.1 micromolar Picogreen, 10 mM CHAPS solution. Measurements were made in a FARCyte fluorescence microplate reader (Amersham Pharmacia, Piscataway, N.J.) using a 485/20 nm excitation filter and a 535/25 emission filter.

A typical multistate affinity ligand-protein interaction assay involved making a 200 microliter mixture containing multistate affinity ligand at a concentration of 100 nM and protein at a concentration of 200 nM, incubating at RT for 30 min., and filtering through the UF well plate under 25 inches of Hg vacuum pressure. The filtrate was collected and triplicate assays for multistate affinity ligand in the filtrate were made with the addition of Picogreen in CHAPS as described above. The concentration of free multistate affinity ligand in the filtrate (LF) was quantified from the standard curves prepared from the same filtration. The concentration of bound multistate affinity ligand (LB) was determined by subtracting the free multistate affinity ligand concentration from the total multistate affinity ligand concentration. Since for the curves presented here the total multistate affinity ligand concentration was 100 nM, the bound concentration (LB) expressed in nanomoles per liter (nM) was calculated as: (LB)=100−(LF). Equilibrium constants were calculated from a single site model:

$${K}_{a} = \frac{( LB )}{( LF ) ( P )} .$$

where (LB) is the concentration of bound multistate affinity ligand, (LF) is the concentration of free multistate affinity ligand and (P) is the concentration of free (unbound) IgG.

Experiments were performed at pH 5, 6, 7 and 8 in various dilutions of buffer containing 150 mM NaCl and 15 mM sodium citrate (“SSC solution”). In undiluted SSC solution, the total concentration of Na⁺ was 165 mM. At two-, four- and ten-fold dilutions of SSC (0.5 SSC, 0.25 SSC and 0.1 SSC, respectively), the sodium ion concentration was as follows:
0.5 SSC (two-fold dilution): 82.5 mM Na⁺
0.25 SSC (four-fold dilution): 41.25 mM Na⁺
0.1 SSC (ten-fold dilution): 16.5 mM Na⁺

Standard curves were measured for human polyclonal IgG binding to the oligonucleotides shown in Table 4. These curves were linear to a good approximation, and were thus used to determine unknown concentrations of oligonucleotide from the filtrate.

TABLE 4
Oligonucleotides used in this study
(members of the primary set of 11 are underlined).

| Name | Sequence | potential conformations | effectors |
|---|---|---|---|
| RAD1 | CCT CTT C/iSp18/CT TCT CC/iSp18/G GAG AAG | YYR triplex HEG linkers | Mg²⁺, pH, NaCI |
| RAD2 | CCT CTT CTT TTT CTT CTC CTT TTT GGA GAA G | YYR triplex | Mg²⁺, pH, NaCI |
| RAD3 | TGG TTG GTT TTT GGA AGG ATT TTT TCC TTC C | RRY triplex/quadruplex | Mg²⁺, KCI, LiCI |
| RAD4 | CCC TCC CTG GGC TTT TTT TGA TTT TTC TTA A | CONTROL | |
| RAD5 | GAG TGA GTC TCA GTT AGT TTC GAT TGA TTC T | CONTROL | |
| RAD6 | GGA AAG GTT TTT GGA AAG GTT TTT CCT TTC C | RRY triplex/quadruplex | Mg²⁺, KCI, LiCI |
| RAD7 | GAG AGA GTT TTT GAG AGA GTT TTT CTC TCT C | RRY triplex | Mg²⁺, NaCI |
| RAD8 | TGG AGT CTG CGC GAG TCA GCG CTC AAG ATC | CONTROL | |
| RAD9 | CTC TCT CTT TTT CTC TCT CTT TTT GAG AGA G | YYR triplex | Mg²⁺, NaCl |
| RAD10 | TGG GCC GGT AAC GGG TTA CCG TAA GGT CCC | 3 way junction/quadruplex | Mg²⁺, KCI, LiCI |
| RAD11 | TGG GCC GGT AAC GGA TTA CCG TAA GGT CCC | 3 way junction/quadruplex | Mg²⁺, KCI, LiCI |
| RAD12 | TTT TCG CGT GTG TGC GCG AA | self-complementary | Mg²⁺, NaCI |
| RAD13 | GGTTGGTTTGGTTGG | quadruplex | KCI, LiCI |
| RAD15 | TTT TCG CGC GTA CGC GCG CGA A | self-complementary | Mg²⁺, NaCI |
| RAD16 | TTT TCG CGC GTT AAC GCG CGA A | self-complementary | Mg²⁺, NaCI |
| RAD19 | TTT IGT TGG TTT GIT TGG | quadruplex | KCI, LiCI |
| RAD20 | CCT CTT CTT TTT CTT CTC | C-rich protonatable | pH, NaCI |
| RAD22 | CGCGAAAACGCG | hairpin | temperature |
| RAD23 | CCT TCC TTT GGA AGG TTG | YR hairpin | temperature |

For each binding determination, 100 nM of oligonucleotide was mixed with 200 nM of protein, and the resultant solution was filtered. The oligonucleotide concentration in the flow-through was used to define the free ligand concentration based on standard linear curves. Each individual data point was the result of 12 measurements: three free ligand concentrations and one data point. The fluorescence in the absence of DNA was determined separately by an average of three additional measurements. The fraction of bound ligand was defined as the free ligand concentration divided by the total ligand concentration (in this case, 100 nM). In the initial studies with this assay using human IgG, determinations were made on a set of 19 ligands shown in Table 4. For the studies with additional IgGs and IgG fragments, determinations were made on a subset of 11 of these ligands. These 11 ligands are underlined in Table 4. The data were analyzed as described above to obtain binding constants. The base 10 logarithms of these binding constants are given in Table 5 for polyclonal human IgG at two different salt concentrations and four different pH values. These results were obtained at 0.25 SSC (41 mM Na⁺) and at 0.5 SSC (82.5 mM Na⁺).

TABLE 5
Screening results expressed as logKa for binding of the
various oligonucleotides to polyclonal human IgG at two
different salt concentrations and four different pHs.

| | 41 mM Na⁺ | | | 82.5 mM Na⁺ | | | | |
|---|---|---|---|---|---|---|---|---|
| name | pH 5 | pH 6 | pH 7 | pH 8 | pH 5 | pH 6 | pH 7 | pH 8 |
| RAD1 | 7.45 | 6.51 | 6.49 | 6.30 | 6.37 | 5.36 | 6.04 | 5.29 |
| RAD2 | 8.74 | 7.61 | 6.96 | 6.57 | 7.04 | 6.28 | 6.23 | 5.75 |
| RAD3 | 8.60 | 7.79 | 6.86 | 6.76 | 7.61 | 6.59 | 6.32 | 5.81 |
| RAD4 | 8.48 | 8.13 | 7.10 | 7.02 | 7.77 | 6.78 | 6.26 | 5.77 |
| RAD5 | 8.75 | 8.03 | 7.11 | 6.81 | 7.71 | 6.69 | 6.26 | 5.80 |
| RAD6 | 8.50 | 7.96 | 6.86 | 6.74 | 7.43 | 6.48 | 6.12 | 5.92 |
| RAD7 | 8.51 | 8.04 | 6.97 | 6.79 | 7.50 | 6.46 | 6.26 | 5.29 |
| RAD8 | 8.41 | 7.85 | 6.86 | 6.84 | 7.24 | 6.33 | 6.07 | 5.48 |
| RAD9 | 8.67 | 7.74 | 7.04 | 6.94 | 7.26 | 6.32 | 6.21 | 5.86 |
| RAD10 | 8.41 | 7.65 | 6.81 | 6.72 | 7.04 | 6.39 | 6.10 | 5.79 |
| RAD11 | 8.31 | 7.64 | 6.85 | 6.85 | 7.06 | 6.29 | 6.17 | 5.70 |
| RAD12 | 7.61 | 6.98 | 6.37 | 5.74 | 6.82 | 6.19 | 5.48 | 6.12 |
| RAD13 | 7.55 | 6.96 | 6.46 | 6.53 | 7.27 | 6.48 | 6.20 | 5.67 |
| RAD15 | 7.65 | 7.00 | 6.33 | 6.19 | 6.80 | 6.06 | 6.23 | 6.25 |
| RAD16 | 8.22 | 7.80 | 7.25 | 6.79 | 7.73 | 6.99 | 6.59 | 6.55 |
| RAD19 | 7.82 | 7.14 | 6.50 | 6.04 | 7.17 | 6.19 | 5.84 | 6.03 |
| RAD20 | 8.11 | 7.98 | 7.40 | 6.64 | 7.85 | 6.33 | 6.17 | 6.22 |
| RAD22 | 5.95 | 6.25 | 6.04 | 5.97 | 5.42 | 5.63 | 5.79 | 5.98 |
| RAD23 | 7.37 | 6.88 | 6.31 | 6.21 | 6.41 | 6.21 | 6.10 | 6.15 |

Shown in Table 6 are the base 10 logarithms of the binding constants vs. pH for binding by the 11 chosen ligands at 41 mM Na⁺ to polyclonal mouse IgG, the Fc and Fab2 fragments of human IgG, the Fab2 fragment of mouse IgG, human IgM, human IgA and human subtypes IgG1, IgG2, IgG3 and IgG4.

TABLE 6
Screening results expressed as logKa for binding to various human and mouse immunoglobulins.

| | Mouse | Human | Human | Mouse | Human | Human | Human | Human | Human | Human |
|---|---|---|---|---|---|---|---|---|---|---|
| | IgG | Fc | Fab2 | Fab2 | IgA | IgM | IgG1 | IgG2 | IgG3 | IgG4 |
| RAD1 | 6.60 | 5.80 | 5.82 | 6.38 | 6.37 | 6.32 | 6.38 | 6.51 | 6.17 | 6.37 |
| RAD2 | 7.49 | 5.86 | 6.45 | 6.71 | 6.90 | 6.73 | 7.04 | 7.06 | 6.64 | 7.08 |
| RAD3 | 7.69 | 6.34 | 6.97 | 7.09 | 7.38 | 6.71 | 7.45 | 7.36 | 6.69 | 7.20 |
| RAD4 | 7.71 | 5.26 | 6.89 | 7.09 | 7.66 | 6.89 | 7.52 | 7.67 | 6.66 | 7.13 |
| RAD7 | 7.69 | 5.99 | 6.68 | 6.97 | 7.33 | 6.89 | 7.30 | 7.25 | 6.74 | 7.16 |
| RAD9 | 7.46 | 5.87 | 6.44 | 6.75 | 6.95 | 6.79 | 7.05 | 7.05 | 6.62 | 7.06 |
| RAD10 | 7.53 | 5.92 | 6.43 | 6.76 | 6.92 | 6.70 | 7.04 | 7.01 | 6.62 | 7.00 |
| RAD15 | 6.35 | negl | 5.33 | 6.21 | 6.23 | 6.19 | 6.39 | 6.18 | 6.22 | 6.13 |
| RAD16 | 7.07 | negl | 6.23 | 6.11 | 6.46 | 6.26 | 6.87 | 6.11 | 6.31 | 6.54 |
| RAD20 | 8.30 | 5.96 | 6.90 | 7.41 | 7.53 | 7.02 | 7.34 | 7.85 | 6.84 | 7.67 |
| RAD23 | 6.77 | negl | 5.64 | 6.33 | 6.38 | 6.08 | 6.14 | 6.86 | 5.62 | 5.97 |

Solution conditions are 37.5 mM NaCl, 3.75 mM sodium citrate, pH 6.0

Salt and pH dependences. The screening assay described here was sensitive and reproducible. Clear differences were discerned among oligonucleotides with respect to their binding to individual immunoglobulins. Differences were also apparent in how individual oligonucleotides bind to different immunoglobulins. The behaviors of the different oligonucleotides with respect to pH-dependent binding showed both quantitative and qualitative differences. Certain oligonucleotides such as RAD20 were seen to be excellent binders to a variety of IgGs and to IgA and IgM, at least under the relatively low salt conditions of these comparative experiments (41 mM Na⁺). Whereas all oligonucleotides showed decreased binding at higher salt, oligonucleotides such as RAD16 showed a reduced salt-dependence compared to others. A characteristic decrease in binding affinity with increased salt concentration is generally observed for DNA-protein interactions, whether specific or nonspecific, and is understood to reflect the entropic consequences of the release of bound cations upon DNA-protein complex formation. It is important to realize that a salt-dependence per se by no means suggests that binding occurs by a nonspecific ion-exchange mechanism. The fact that a wide variation of binding strength is observed among oligonucleotides for binding to a particular type of IgG, IgA or IgM and that the ordering of oligonucleotide binding depends on the nature of the immunoglobulin further demonstrates specific interactions with specific immunoglobulin surface features. In general, a decrease in binding constant is anticipated as the pH is increased. This effect is anticipated even for highly specific binding interactions, as long as ionic interactions occur between negatively charged groups on the DNA and positively charged groups on the protein. However, the pH effect is less uniform than the salt effect and can reflect protonation events near the binding site on both the protein and on the DNA. For DNA, cytosine bases can protonate and allow the formation of fold-back and tetraplex structures around neutral pH, which can significantly affect the pH-dependent binding curves. It is notable that there are a number of situations where the fraction of bound ligand does not change greatly between pH 6 and 7 and even a few cases where the binding of individual oligonucleotides appears to increase on going from pH 6 to pH 7.

Location of the multistate affinity ligand binding sites. Binding to the Fc fragment was significant only at the lowest pH examined. In contrast, both the human and the mouse Fab2 fragments showed binding that is comparable to that observed for whole IgG as well as similar dependences on multistate affinity ligand type and on pH. Based on these results, it seems likely that the polyanion binding sites on IgG that were recognized by the multistate affinity ligands were primarily located on the Fab2 fragment.

Binding to human IgG subtypes. As can be discerned from the results shown, the protein subtype that binds most tightly to a number of the multistate affinity ligands was, in fact, subtype IgG2. In contrast, IgG2 scarcely binds to Protein A, which places significant limitations on the purification of IgG2 subtypes. The other subtypes likewise bound tightly to several of the multistate affinity ligands, although the ordering of multistate affinity ligand binding did not appear to depend on the IgG subtype examined.

Binding to IgA and IgM. IgA bound very tightly to RAD4, RAD20, RAD3 and RAD23. The binding of IgM showed a lower level of discrimination among the tightest binding multistate affinity ligands under the solution conditions studied, although this discrimination may be enhanced by variations in binding and elution conditions.

### Example 29

### Screening of Multistate Affinity Ligands for Binding to Immunoglobulins

100 to 150 nanomoles of amino-linked TAL was reacted with 300 uL of NHS-activated Sepharose according to the manufacturer's procedure. After overnight coupling, unreacted sites on the Sepharose were deactivated by reaction with 0.5 M ethanolamine. OD measurements after removing the released NHS (which interferes with the OD measurements) by gel filtration indicated TAL substitution to the Sepharose was between 120 nanomoles (RAD 4) to 70 nanomoles (RAD 16), meaning a degree of substitution on the TAL-Sepharose of approximately 0.3 micromole/ml of gel. The TAL-Sepharose was divided equally between three Costar centrifuge tubes with wells containing 22 micron filters (approx. 100 microliters Sepharose per well). The gel in each tube was equilibrated with the appropriate buffer by addition of multiple washes with buffer followed by spinning the buffer through the gel (which was retained on the filter in the wells). Two microliters of fluorescein-labeled IgG (Jackson ImmunoResearch Laboratories, West Grove, Pa.) at a concentration of 2 mg/ml in a solution containing BSA (15 mg/ml) was added to 200 microliters of the appropriate buffer solution, and the reaction mixture was then added to the gel-containing wells. The gel and IgG were mixed on a shaker for 1 hour, and the solution was recovered by spinning it through the gel. Solution fluorescence was measured in a fluorescence plate counter (where low readings in the filtrate indicate binding to the TAL-Sepharose). A blank was done by filtering the same solution through a filter well with no gel. Results are presented in Table 7 below for the following buffers: 0.0067M phosphate, pH 7.4 with 0.15M NaCl (PBS); 0.067 M phosphate, pH 7.4 with 1.5M NaCl (10×PBS); and 0.020M MES pH5.8 (MES).

TABLE 7
Binding of fluorescein-labeled human IgG to immobilized TALs under
varying buffer conditions (expressed as counts per second, where
binding is determined by subtracting counts from blank)

| | Mouse | Human | Human | Mouse | Human | Human | Human | Human | Human | Human |
|---|---|---|---|---|---|---|---|---|---|---|
| | IgG | Fc | Fab2 | Fab2 | IgA | IgM | IgG1 | IgG2 | IgG3 | IgG4 |
| RAD1 | 6.60 | 5.80 | 5.82 | 6.38 | 6.37 | 6.32 | 6.38 | 6.51 | 6.17 | 6.37 |
| RAD2 | 7.49 | 5.86 | 6.45 | 6.71 | 6.90 | 6.73 | 7.04 | 7.06 | 6.64 | 7.08 |
| RAD3 | 7.69 | 6.34 | 6.97 | 7.09 | 7.38 | 6.71 | 7.45 | 7.36 | 6.69 | 7.20 |
| RAD4 | 7.71 | 5.26 | 6.89 | 7.09 | 7.66 | 6.89 | 7.52 | 7.67 | 6.66 | 7.13 |
| RAD7 | 7.69 | 5.99 | 6.68 | 6.97 | 7.33 | 6.89 | 7.30 | 7.25 | 6.74 | 7.16 |
| RAD9 | 7.46 | 5.87 | 6.44 | 6.75 | 6.95 | 6.79 | 7.05 | 7.05 | 6.62 | 7.06 |
| RAD10 | 7.53 | 5.92 | 6.43 | 6.76 | 6.92 | 6.70 | 7.04 | 7.01 | 6.62 | 7.00 |
| RAD15 | 6.35 | negl | 5.33 | 6.21 | 6.23 | 6.19 | 6.39 | 6.18 | 6.22 | 6.13 |
| RAD16 | 7.07 | negl | 6.23 | 6.11 | 6.46 | 6.26 | 6.87 | 6.11 | 6.31 | 6.54 |
| RAD20 | 8.30 | 5.96 | 6.90 | 7.41 | 7.53 | 7.02 | 7.34 | 7.85 | 6.84 | 7.67 |
| RAD23 | 6.77 | negl | 5.64 | 6.33 | 6.38 | 6.08 | 6.14 | 6.86 | 5.62 | 5.97 |

For the purposes of clarity and understanding, the present invention has been described in the foregoing examples and disclosure. It will be apparent, however, that certain changes and modifications mat be practiced within the scope of the appended claims.

### REFERENCES

1. Anselmet, A., Mayat, E., Wietek, S., Layer, P. G., Payrastre, B., and Massoulie, J. (2002). Non-antisense cellular responses to oligonucleotides. FEBS Lett 510, 175-180.
2. Beal, P. A., and Dervan, P. B. (1991). Second structural motif for recognition of DNA by oligonucleotide-directed triple-helix formation. Science 251, 1360-1363.
3. Beal, P. A., and Dervan, P. B. (1992). The influence of single base triplet changes on the stability of a pur.pur.pyr triple helix determined by affinity cleaving. Nucleic Acids Res 20, 2773-2776.
4. Benight, A. S., Wang, Y. S., Amaratunga, M., Chattopadhyaya, R., Henderson, J., Hanlon, S., and Ikuta, S. (1989). Conformation and dynamics of a left-handed Z-DNA hairpin: studies of d(CGCGCGTTTTCGCGCG) in solution. Biochemistry 28, 3323-3332.
5. Bonnet, G., Tyagi, S., Libchaber, A., and Kramer, F. R. (1999). Thermodynamic basis of the enhanced specificity of structured DNA probes. Proc Natl Acad Sci USA 96, 6171-6176.
6. Braunlin, W., Xu, Q., Hook, P., Fitzpatrick, R., Klinger, J. D., Burlier, R., and Kurtz, C. B. (2004). Toxin Binding of Tolevamer, a Polyanionic Drug that Protects against Antibiotic-Associated Diarrhea. Biophysical Journal 87.
7. Burge, S., Parkinson, G. N., Hazel, P., Todd, A. K., and Neidle, S. (2006). Quadruplex DNA: sequence, topology and structure. Nucleic Acids Res 34, 5402-5415.
8. Chen, C. J., Chen, S., Woodbury, C. P., Jr., and Venton, D. L. (1998). Pulsed ultrafiltration characterization of binding. Anal Biochem 261, 164-182.
9. Cogoi, S., Quadrifoglio, F., and Xodo, L. E. (2004). G-rich oligonucleotide inhibits the binding of a nuclear protein to the Ki-ras promotor and strongly reduces cell growth in human carcinoma pancreatic cells. Biochemistry 43, 2512-2523.
10. Dapic, V., Abdomerovic, V., Marrington, R., Peberdy, J., Rodger, A., Trent, J. O., and Bates, P. J. (2003). Biophysical and biological properties of quadruplex oligodeoxyribonucleotides. Nucleic Acids Res 31, 2097-2107.
11. Dapic, V., Bates, P. J., Trent, J. O., Rodger, A., Thomas, S. D., and Miller, D. M. (2002). Antiproliferative activity of G-quartet-forming oligonucleotides with backbone and sugar modifications. Biochemistry 41, 3676-3685.
12. Demidov, V. V., and Frank-Kamenetskii, M. D. (2004). Two sides of the coin: affinity and specificity of nucleic acid interactions. Trends Biochem Sci 29, 62-71.
13. Duquette, M. L., Handa, P., Vincent, J. A., Taylor, A. F., and Maizels, N. (2004). Intracellular transcription of G-rich DNAs induces formation of G-loops, novel structures containing G4 DNA. Genes Dev 18, 1618-1629.
14. Eaton, B. E., Gold, L., and Zichi, D. A. (1995). Let's get specific: the relationship between specificity and affinity. Chem Biol 2, 633-638.
15. Felsenfeld, G., and Rich, A. (1957). Studies on the formation of two- and three-stranded polyribonucleotides. Biochim Biophys Acta 26, 457-468.
16. Fishel, R., Anziano, P., and Rich, A. (1990). Z-DNA affinity chromatography. Methods Enzymol 184, 328-340.
17. Fountoulakis, M., and Takacs, B. (1998). Design of protein purification pathways: application to the proteome of Haemophilus influenzae using heparin chromatography. Protein Expr Purif 14, 113-119.
18. Fountoulakis, M., and Takacs, B. (2002). Enrichment and proteomic analysis of low-abundance bacterial proteins. Methods Enzymol 358, 288-306.
19. Fountoulakis, M., Takacs, B., and Langen, H. (1998). Two-dimensional map of basic proteins of Haemophilus influenzae. Electrophoresis 19, 761-766.
20. Fu, W., Gorelick, R. J., and Rein, A. (1994). Characterization of human immunodeficiency virus type 1 dimeric RNA from wild-type and protease-defective virions. J Virol 68, 5013-5018.
21. Giovannangeli, C., Rougee, M., Garestier, T., Thuong, N. T., and Helene, C. (1992). Triple-helix formation by oligonucleotides containing the three bases thymine, cytosine, and guanine. Proc Natl Acad Sci USA 89, 8631-8635.
22. Goni, J. R., Vaquerizas, J. M., Dopazo, J., and Orozco, M. (2006). Exploring the reasons for the large density of triplex-forming oligonucleotide target sequences in the human regulatory regions. BMC Genomics 7, 63.
23. Griffin, L. C., and Dervan, P. B. (1989). Recognition of thymine adenine.base pairs by guanine in a pyrimidine triple helix motif. Science 245, 967-971.
24. Hanakahi, L. A., Sun, H., and Maizels, N. (1999). High affinity interactions of nucleolin with G-G-paired rDNA. J Biol Chem 274, 15908-15912.
25. Hardin, C. C., Perry, A. G., and White, K. (2000). Thermodynamic and kinetic characterization of the dissociation and assembly of quadruplex nucleic acids. Biopolymers 56, 147-194.
26. Hill, D. J., Mio, M. J., Prince, R. B., Hughes, T. S., and Moore, J. S. (2001). A field guide to foldamers. Chem Rev 101, 3893-4012.
27. Hoyne, P. R., Gacy, A. M., McMurray, C. T., and Maher, L. J., 3rd (2000). Stabilities of intrastrand pyrimidine motif DNA and RNA triple helices. Nucleic Acids Res 28, 770-775.
28. Jing, N., Li, Y., Xu, X., Sha, W., Li, P., Feng, L., and Tweardy, D. J. (2003). Targeting Stat3 with G-quartet oligodeoxynucleotides in human cancer cells. DNA Cell Biol 22, 685-696.
29. Jones, L. S., Yazzie, B., and Middaugh, C. R. (2004). Polyanions and the proteome. Mol Cell Proteomics.
30. Langen, H., Takacs, B., Evers, S., Berndt, P., Lahm, H. W., Wipf, B., Gray, C., and Fountoulakis, M. (2000). Two-dimensional map of the proteome of Haemophilus influenzae. Electrophoresis 21, 411-429.
31. Lomakin, A., and Frank-Kamenetskii, M. D. (1998). A theoretical analysis of specificity of nucleic acid interactions with oligonucleotides and peptide nucleic acids (PNAs). J Mol Biol 276, 57-70.
32. Mori, T., Oguro, A., Ohtsu, T., and Nakamura, Y. (2004). RNA aptamers selected against the receptor activator of NF-kappaB acquire general affinity to proteins of the tumor necrosis factor receptor family. Nucleic Acids Res 32, 6120-6128.
33. Ng, H. L., Kopka, M. L., and Dickerson, R. E. (2000). The structure of a stable intermediate in the A<-->B DNA helix transition. Proc Natl Acad Sci USA 97, 2035-2039.
34. Olivas, W. M., and Maher, L. J., 3rd (1995). Competitive triplex/quadruplex equilibria involving guanine-rich oligonucleotides. Biochemistry 34, 278-284.
35. Ono, A., Chen, C. N., and Kan, L. S. (1991). DNA triplex formation of oligonucleotide analogues consisting of linker groups and octamer segments that have opposite sugar-phosphate backbone polarities. Biochemistry 30, 9914-9912.
36. Padmanabhan, K., Padmanabhan, K. P., Ferrara, J. D., Sadler, J. E., and Tulinsky, A. (1993). The structure of alpha-thrombin inhibited by a 15-mer single-stranded DNA aptamer. J Biol Chem 268, 17651-17654.
37. Paeschke, K., Simonsson, T., Postberg, J., Rhodes, D., and Lipps, H. J. (2005). Telomere end-binding proteins control the formation of G-quadruplex DNA structures in vivo. Nat Struct Mol Biol 12, 847-854.
38. Plum, G. E., Park, Y. W., Singleton, S. F., Dervan, P. B., and Breslauer, K. J. (1990). Thermodynamic characterization of the stability and the melting behavior of a DNA triplex: a spectroscopic and calorimetric study. Proc Natl Acad Sci USA 87, 9436-9440.
39. Pohl, F. M., and Jovin, T. M. (1972). Salt-induced co-operative conformational change of a synthetic DNA: equilibrium and kinetic studies with poly (dG-dC). J Mol Biol 67, 375-396.
40. Rich, A., and Zhang, S. (2003). Timeline: Z-DNA: the long road to biological function. Nat Rev Genet. 4, 566-572.
41. Schade, M., Behlke, J., Lowenhaupt, K., Herbert, A., Rich, A., and Oschkinat, H. (1999). A 6 by Z-DNA hairpin binds two Z alpha domains from the human RNA editing enzyme ADAR1. FEBS Lett 458, 27-31.
42. Schultze, P., Macaya, R. F., and Feigon, J. (1994). Three-dimensional solution structure of the thrombin-binding DNA aptamer d(GGTTGGTGTGGTTGG). J Mol Biol 235, 1532-1547.
43. Semerad, C. L., and Maher, L. J., 3rd (1994). Exclusion of RNA strands from a purine motif triple helix. Nucleic Acids Res 22, 5321-5325.
44. Shafer, R. H., and Smirnov, I. (2000). Biological aspects of DNA/RNA quadruplexes. Biopolymers 56, 209-227.
45. Shefcheck, K., Yao, X., and Fenselau, C. (2003). Fractionation of cytosolic proteins on an immobilized heparin column. Anal Chem 75, 1691-1698.
46. Siddiqui-Jain, A., Grand, C. L., Bearss, D. J., and Hurley, L. H. (2002). Direct evidence for a G-quadruplex in a promoter region and its targeting with a small molecule to repress c-MYC transcription. Proc Natl Acad Sci USA 99, 11593-11598.
47. Simonsson, T. (2001). G-quadruplex DNA structures—variations on a theme. Biol Chem 382, 621-628.
48. Simonsson, T., Pecinka, P., and Kubista, M. (1998). DNA tetraplex formation in the control region of c-myc. Nucleic Acids Res 26, 1167-1172.
49. Smirnov, I. V., and Shafer, R. H. (2007). Electrostatics dominate quadruplex stability. Biopolymers 85, 91-101.
50. Ueberle, B., Frank, R., and Herrmann, R. (2002). The proteome of the bacterium Mycoplasma pneumoniae: comparing predicted open reading frames to identified gene products. Proteomics 2, 754-764.
51. Utt, M., Nilsson, I., Ljungh, A., and Wadstrom, T. (2002). Identification of novel immunogenic proteins of Helicobacter pylori by proteome technology. J Immunol Methods 259, 1-10.
52. Van Dyke, M. W., Nelson, L. D., Weilbaecher, R. G., and Mehta, D. V. (2004). Stm1p, a G4 quadruplex and purine motif triplex nucleic acid-binding protein, interacts with ribosomes and subtelomeric Y′ DNA in Saccharomyces cerevisiae. J Biol Chem 279, 24323-24333.
53. Vater, A., and Klussmann, S. (2003). Toward third-generation aptamers: Spiegelmers and their therapeutic prospects. Curr Opin Drug Discov Devel 6, 253-261.
54. Wahl, M. C., and Sundaralingam, M. (1997). Crystal structures of A-DNA duplexes. Biopolymers 44, 45-63.
55. Wang, A. H., Quigley, G. J., Kolpak, F. J., Crawford, J. L., van Boom, J. H., van der Marel, G., and Rich, A. (1979). Molecular structure of a left-handed double helical DNA fragment at atomic resolution. Nature 282, 680-686.
56. Wang, S., and Kool, E. T. (1995). Relative stabilities of triple helices composed of combinations of DNA, RNA and 2′-O-methyl-RNA backbones: chimeric circular oligonucleotides as probes. Nucleic Acids Res 23, 1157-1164.
57. Watson, J. D., and Crick, F. H. (1953). Molecular structure of nucleic acids; a structure for deoxyribose nucleic acid. Nature 171, 737-738.
58. Xu, Q., Jampani, S. R., Deng, H., and Braunlin, W. H. (1995). Chiral recognition of deoxyoligonucleotides by delta- and lambda-tris(ethylenediamine)cobalt(III). Biochemistry 34, 14059-14065.
59. Xu, Q., Shoemaker, R. K., and Braunlin, W. H. (1993). Induction of B-A transitions of deoxyoligonucleotides by multivalent cations in dilute aqueous solution. Biophys J 65, 1039-1049.
60. Xu, X., Hamhouyia, F., Thomas, S. D., Burke, T. J., Girvan, A. C., McGregor, W. G., Trent, J. O., Miller, D. M., and Bates, P. J. (2001). Inhibition of DNA replication and induction of S phase cell cycle arrest by G-rich oligonucleotides. J Biol Chem 276, 43221-43230.

We claim:

1. A medium for separating a target substance from a mixture of substances, said medium comprising a nucleotide-containing tunable affinity ligand within a reaction mixture, said tunable affinity ligand existing in a first conformational state having a quantifiable first affinity for the target substance under a first set of reaction conditions and a second conformational state having a quantifiable second affinity for the target substance under a second set of reaction conditions wherein the first affinity is measurably different from the second affinity.

2. The medium of claim 1 wherein the reaction mixture comprises a matrix selected from the group consisting of a gel, a sol, a suspension, a polymer, a coating, a nanoparticle, a microparticle, a vesicle, a solid, semisolid, insoluble, insolubilized, precipitable, porous or nonporous support, a glass, a bead, a resin, a colloid, a membrane and a filter.

3. The medium of claim 1 wherein the target substance comprises at least one of a molecule, a multimolecular complex, a particle, a virus, a pathogen, a microorganism, a cell or a subcellular organelle.

4. The medium of claim 3 wherein the molecule is selected from the group consisting of inorganic molecules, organic molecules, proteins, peptides, lipids, carbohydrates, drugs, pharmacophores, hormones, receptors, vitamins, toxins and congeners and conjugates thereof.

5. The medium of claim 1 wherein the tunable affinity ligand comprises a nonnaturally occurring polymer.

6. The medium of claim 1 wherein the tunable affinity ligand is prepared at least in part by solid phase synthesis.

7. The medium of claim 1 wherein the first conformational state and the second conformational state can be distinguished by at least one of a physical, spectroscopic, hydrodynamic, calorimetric, thermodynamic, electrophoretic, chromatographic, biological or computational technique.

8. The medium of claim 1 wherein the first affinity or the second affinity for the target substance is quantifiably dependent on the presence or amount of at least one nontarget substance selected from the group consisting of salts, sugars, hydrogen ions, monovalent ions, multivalent ions, zwitterions, chelating agents, detergents, nucleotides, catalysts, cofactors, intercalating agents and dyes.

9. The medium of claim 1 wherein the tunable affinity ligand comprises at least one sequence of nucleotides that participates in complementary base pairing to form an intramolecular duplex in at least one of the first conformational state or the second conformational state.

10. The medium of claim 1 wherein the tunable affinity ligand is a nondenaturing tunable affinity ligand.

11. A device for isolating target substances from a sample, said device comprising:
a) a nucleotide-containing tunable affinity ligand capable of existing in a target-binding state and a target-nonbinding state;
b) means for delivering the sample to the tunable affinity ligand to form a reaction mixture in which the tunable affinity ligand exists in the target-binding state;
c) means for partitioning ligand-target complexes from other substances in the reaction mixture;
d) means for converting the tunable affinity ligand from the target-binding state to the target-nonbinding state; and
e) means for partitioning unbound target molecules from ligand-bound target molecules.

12. The device of claim 11 wherein the tunable affinity ligand comprises at least one sequence of nucleotides that participates in complementary base pairing to form an intramolecular duplex in at least one of the first conformational state or the second conformational state.

13. The device of claim 11 wherein the tunable affinity ligand is a nondenaturing tunable affinity ligand.

14. A kit for separating a target substance from a sample, said kit comprising a buffer-responsive nucleotide-containing tunable affinity ligand, a binding buffer and a releasing buffer wherein the tunable affinity ligand switches between a target-binding state in the presence of the binding buffer and a target-nonbinding state in the presence of the releasing buffer.

15. The kit of claim 14 wherein the tunable affinity ligand comprises at least one sequence of nucleotides that participates in complementary base pairing to form an intramolecular duplex in at least one of the first conformational state or the second conformational state.

16. The kit of claim 14 wherein the tunable affinity ligand is a nondenaturing tunable affinity ligand.

17. A system for separating a target substance from a sample, said system comprising:
a) a processing reservoir containing a separation reagent;
b) input means for delivering the sample to the processing reservoir;
c) output means for removing the target substance from the processing reservoir;
d) a first buffer solution; and
e) a second buffer solution;
wherein the separation reagent is a nucleotide-containing tunable affinity ligand that exists in a first conformational state having a quantifiable first affinity for the target substance under a first set of reaction conditions and a second conformational state having a quantifiable second affinity for the target substance under a second set of reaction conditions wherein the first affinity is measurably different from the second affinity.

18. The system of claim 17 wherein the tunable affinity ligand comprises at least one sequence of nucleotides that participates in complementary base pairing to form an intramolecular duplex in at least one of the first conformational state or the second conformational state.

19. The system of claim 17 wherein the tunable affinity ligand is a nondenaturing tunable affinity ligand.

20. A method of purifying a target substance from a sample, said method comprising:
a) contacting the sample with an environmentally-sensitive nucleotide-containing tunable affinity ligand under a first environmental condition under which the tunable affinity ligand binds to the target substance to form a ligand-target complex;
b) partitioning the ligand-target complex from nontarget substances in the sample; and
c) releasing the target substance from the ligand-target complex by exposing the ligand-target complex to a second environmental condition
wherein
i) the tunable affinity ligand reversibly partitions between a first conformational state having a first affinity for the target substance under the first environmental condition and a second conformational state having a second affinity for the target substance under the second environmental condition; and
ii) the first affinity is measurably different from the second affinity.

21. The method of claim 20 wherein the tunable affinity ligand comprises at least one sequence of nucleotides that participates in complementary base pairing to form an intramolecular duplex in at least one of the first conformational state or the second conformational state.

22. The method of claim 20 wherein the tunable affinity ligand is a nondenaturing tunable affinity ligand.

23. A method of separating a first substance in a sample from a second substance in the sample, said method comprising:
a) contacting the sample with a nucleotide-containing tunable affinity ligand immobilized on a support immersed in a binding buffer;
b) incubating the sample with the immobilized tunable affinity ligand for a sufficient contact time to allow the immobilized tunable affinity ligand to bind the first substance to form an immobilized ligand-substance complex;
c) performing a rinsing step to remove the second substance;
d) performing at least one elution step to dissociate the first substance from the ligand of the immobilized ligand-substance complex; and
e) collecting at least one product of the at least one elution step;
wherein
i) said at least one product comprises the first substance; and
ii) said at least one elution step causes the tunable affinity ligand to shift from a first conformational state that favors association of immobilized ligand-substance complexes to a second conformational state that favors dissociation of immobilized ligand-substance complexes.

24. The method of claim 23 further rinsing the support with a cleaning buffer.

25. The method of claim 23 further comprising rinsing the support with a buffer that restores the tunable affinity ligand to the first conformational state.

26. The method of claim 23 further comprising rinsing the support in a storage buffer.

27. The method of claim 23

28. The method of claim 23 wherein the tunable affinity ligand comprises at least one sequence of nucleotides that participates in complementary base pairing to form an intramolecular duplex in at least one of the first conformational state or the second conformational state.

29. The method of claim 23 wherein the tunable affinity ligand is a nondenaturing tunable affinity ligand.

30. A separation medium comprising a support-bound plurality of ligands including at least a first ligand and a second ligand, said first ligand being a nucleotide-containing tunable affinity ligand existing in a first state having a quantifiable first affinity for a target substance under a first set of conditions and a second state having a quantifiable second affinity for the target substance under a second set of conditions wherein the first ligand is structurally different from the second ligand.

31. The medium of claim 30 wherein the plurality of ligands includes ligands having different affinities for the target substance.

32. The medium of claim 30 wherein the plurality of ligands includes ligands having different specificities for the target substance.

33. The medium of claim 30 wherein the plurality of ligands includes ligands that specifically bind different target substances.

34. The medium of claim 30 wherein the tunable affinity ligand comprises at least one sequence of nucleotides that participates in complementary base pairing to form an intramolecular duplex in at least one of the first conformational state or the second conformational state.

35. The medium of claim 30 wherein the tunable affinity ligand is a nondenaturing tunable affinity ligand.

36. A reagent for detecting a target substance, said reagent a comprising a nucleotide-containing tunable affinity ligand capable of existing in a first conformational state having a quantifiable first affinity for the target substance under a first set of reaction conditions and a second conformational state having a quantifiable second affinity for the target substance under a second set of reaction conditions wherein the first affinity is measurably different from the second affinity.

37. The reagent of claim 36 wherein the tunable affinity ligand comprises at least one sequence of nucleotides that participates in complementary base pairing to form an intramolecular duplex in at least one of the first conformational state or the second conformational state.

38. The reagent of claim 36 wherein the tunable affinity ligand comprises at least one of a normucleotide spacer or a normucleotide linker.

39. The reagent of claim 36 wherein the tunable affinity ligand is a nondenaturing tunable affinity ligand.

40. The reagent of claim 36 wherein the tunable affinity ligand is capable of reversibly switching between the first affinity state and the second affinity state.

41. A sensor for detecting a target substance, said sensor comprising a ligand functionally connected to a transducer, said ligand being a nucleotide-containing tunable affinity ligand capable of existing in a first conformational state having a quantifiable first affinity for the target substance under a first set of reaction conditions and a second conformational state having a quantifiable second affinity for the target substance under a second set of reaction conditions wherein the first affinity is measurably different from the second affinity.

42. The sensor of claim 41 wherein the tunable affinity ligand comprises at least one sequence of nucleotides that participates in complementary base pairing to form an intramolecular duplex in at least one of the first conformational state or the second conformational state.

43. The sensor of claim 41 wherein the tunable affinity ligand comprises at least one of a normucleotide spacer or a normucleotide linker.

44. The sensor of claim 41 wherein the tunable affinity ligand is a nondenaturing tunable affinity ligand.

45. The sensor of claim 41 wherein said detecting a target substance includes monitoring time-dependent changes in the presence or amount of the target substance.

46. A method for detecting the presence of a target substance comprising:
a) contacting the target substance with nucleotide-containing tunable affinity ligands in a reaction mixture under a first set of conditions that favors a target-nonbinding conformation of the tunable affinity ligands;
b) exposing the reaction mixture to a second set of conditions that favors a target-binding conformation of the tunable affinity ligands to form target-bound tunable affinity ligand-receptor complexes; and
b) detecting a difference in the conformation, properties or affinity state of at least one of the tunable affinity ligands or the tunable affinity ligand-receptor complexes in the target-bound state compared with the target-unbound state.

47. A kit comprising the reagent of claim 36.

48. A kit comprising the sensor of claim 41.
